(19) United States
(12) Patent Application Publication
(10) Pub. No.: US 2024/0238254 A1
(43) Pub. Date: Jul. 18, 2024

(54) PHARMACOLOGICAL INTERVENTION OF THE ARACHIDONIC ACID PATHWAY TO CURE AMYOTROPHIC LATERAL SCLEROSIS

(71) Applicants: The Johns Hopkins University, Baltimore, MD (US); The University of Southern California, Los Angeles, CA (US)

(72) Inventors: Gabsang LEE, Ellicott City, MD (US); Hyungjin EOH, Baltimore, MD (US); Hojae LEE, Baltimore, MD (US); Thomas LLOYD, Baltimore, MD (US); Nicholas J. MARAGAKIS, Baltimore, MD (US)

(73) Assignees: The Johns Hopkins University, Baltimore, MD (US); The University of Southern California, Los Angeles, CA (US)

(21) Appl. No.: 18/563,800

(22) PCT Filed: May 24, 2022
(86) PCT No.: PCT/US2022/030773

§ 371 Date: Nov. 22, 2023

Related U.S. Application Data
(60) Provisional application No. 63/192,284, filed on May 24, 2021.
(60) Provisional application No. 63/278,779, filed on Nov. 12, 2021.

(51) Int. Cl. A61K 31/415; A61K 31/05; A61K 31/192; A61K 31/352; A61P 25/28; C12N 5/0793; C12Q 1/6809; C12Q 1/6883

(52) U.S. Cl. CPC A61K 31/415; A61K 31/05; A61K 31/192; A61K 31/352; A61P 25/28; C12N 5/0619; C12Q 1/6809; C12Q 1/6883; C12N 2500/38; C12N 2501/13; C12N 2501/41; C12N 2501/999; C12N 2506/02; C12N 2506/45; C12Q 2600/158

(57) ABSTRACT

Embodiments disclosed relate to methods of treating amyotrophic lateral sclerosis (ALS), including: selecting a therapeutic compound: treating an aberrant arachidonic acid (AA) metabolic pathway in an ALS cell.

This application claims priority to U.S. Provisional Application No. 63/192,284, filed May 24, 2021 and U.S. Provisional Application No. 63/278,779, filed Nov. 12, 2021, the contents of which are incorporated herein by reference in their entirety.

### GOVERNMENT SUPPORT

This invention was made with government support under R01NS093213 and AI143870 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 24, 2022, is named 02240_561481_SL.txt and is 24,350 bytes in size.

### BACKGROUND

### 1. Technical Field

The field of the currently claimed embodiments of this invention relate to methods of treating amyotrophic lateral sclerosis (ALS) including: selecting a therapeutic compound: treating an aberrant arachidonic acid (AA) metabolic pathway in an ALS cell.

### 2. Discussion of Related Art

Progressive loss of spinal motor neurons (sMN) is a hallmark of amyotrophic lateral sclerosis (ALS)¹⁻³, which causes progressive paralysis and eventually death⁴. Around 10% of ALS cases are inherited, and over 20 genes are now known to cause familial ALS (fALS) when mutated such as superoxide dismutase 1 (SOD1)², C9ORF72⁵, TAR DNA binding protein (TDP43)⁶, fused in sarcoma (FUS)⁷, optineurin (OPTN)⁸, profilin1 (PFNI)⁹, matrin-3 (MATR3)¹⁰, Tubulin Alpha 4A (TUBA4A)¹¹, TANK binding kinase1 (TBK1)¹² and several others.¹³. Much of the current understanding of ALS pathogenesis has been made through investigations of animal models carrying mutations in genes such as SOD1, C9ORF72 and TDP43^{14,15}, and these models provide opportunities to test therapeutic targets. However, 90% of ALS cases are sporadic (sALS) and caused by unknown factors^{13,16}. Recently, human induced pluripotent stem cells (hiPSCs) have emerged as an alternative and complementary system to animal models¹⁷⁻²¹. One of the advantages of hiPSC systems is enabling the generation of personalized cellular models with patient-specific mutations and genetic backgrounds. Using this technique, ALS cellular models have been generated without complicated genetic modifications for fALS cases, as well as for sALS. These hiPSC-based ALS cellular models have been used to elucidate pathogenic molecular mechanisms in ALS²²⁻²⁶ by comparing ALS-specific sMN and healthy sMN, although the healthy control hiPSCs have variable genetic backgrounds. Recently, genetically corrected isogenic control hiPSCs have been proposed as an ideal control using newly developed gene editing technology^{25,27,28} because the isogenic hiPSCs could minimize genetic variations in multiple healthy control hiPSCs. Nevertheless, an isogenic control is not feasible in cases with multiple and/or unknown mutations or in sporadic cases. Unsurprisingly, some reports have suggested that the widely-used CRISPR-Cas9 system may cause inadvertent DNA changes that could result in unintended phenotypes irrelevant to disease^{29,30}. Thus, the new concept of comparative disease modeling using hiPSCs may lead to new insights into underlying ALS disease mechanisms.

Clinical features of ALS patients show selective vulnerability depending upon the specific motor neuron subtypes. Even in late stages of ALS disease progression, ocular motor neuron (oMN) is functionally intact in most patients. While motor neuron subtypes in the spinal cord, hindbrain and cortex are gradually impaired or lost during ALS progression leading to disability of voluntary movement, eye movements controlled by oMNs in the midbrain remain relatively unaffected until the final stage³¹⁻³⁴. These findings suggest that there might be an intrinsic resistant mechanism underlying oMN-specific tolerance against ALS pathogenesis, and comparative studies between sMN and oMN could help identify the mechanistic basis underlying selective susceptibility in ALS. To this end, oMN have been studied as an ALS-resistant cell population³⁵⁻³⁸. Kaplan and colleagues compared differentially expressed genes in oMNs and sMNs of wildtype (WT) postnatal mice and found that matrix metalloproteinase-9 (MMP-9) is a relevant gene for neurodegeneration in fast motor neurons of a SOD1 ALS mouse model³⁵. Unfortunately, this has not yet been studied or tested with human ALS neuronal subtypes, primarily because isolation of oMNs is too challenging to perform in human patients. Allodi and colleagues also suggested that IGF2 (Insulin-like growth factor 2) has a protective role in SOD1 mouse model and ALS sMN in vitro, however, it is unknow whether it is also found in other mutations or sporadic cases of ALS. (Allodi 2019) Moreover, any given studies with already degenerated sMNs in ALS patients may not be adequate to uncover a primary cell intrinsic causality of ALS pathogenesis.

Many studies have attempted to understand different aspects of ALS pathogenesis and have identified mitochondrial dysfunction³⁹, excitotoxicity^{40,41} and astrocyte induced non-cell autonomous effects^{42,43} as potential mechanisms. However, one understudied hypothesis is that ALS disease might be associated with alterations in lipid metabolism. Previous studies reported abnormal levels of ceramide⁴⁴, cholestenoic acids⁴⁵, cholesterol or low density lipoprotein (LDL)/high density lipoprotein (HDL) in biofluids of ALS patients⁴⁶. However, these reports focused on the role of specific genes involved in lipid metabolism using a rodent model, or simply showed alterations of specific metabolites in patient cerebrospinal fluid (CSF). Although these publications have provided interesting data that allow the speculation of a relationship between ALS pathology and aberrant lipid metabolism, more dynamic understanding of lipid metabolic dysregulation in ALS-specific human sMNs must be performed to elucidate the metabolic causality of altered lipid metabolism in ALS disease.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a method of treating an amyotrophic lateral sclerosis (ALS) cell including: selecting a therapeutic compound: treating an aberrant arachidonic acid (AA) metabolic pathway in the ALS cell including contacting the ALS cell with the therapeutic compound.

An embodiment of the invention relates to a method of treating a subject with ALS including: selecting a therapeutic compound: and treating an aberrant arachidonic acid (AA) metabolic pathway in the subject including administering to the subject the therapeutic compound.

An embodiment of the invention relates to a method of differentiating a human stem cell to an ocular motor neuron (oMN) ALS-specific human cell type, including: culturing the human stem cell in a first media including recombinant sonic hedgehog signaling protein and purmorphamine for 9 days: culturing the human stem cell in a second media including brain-derived neurotrophic factor (BDNF), glial cell line-derived neurotrophic factor (GDNF), and Ascorbic Acid for at least 1 day. In such an embodiment, the second media does not include sonic hedgehog signaling protein or purmorphamine.

An embodiment of the invention relates to an ocular motor neuron (oMN) ALS-specific human cell generated from the method discussed above.

An embodiment of the invention relates to a method for identifying whether a metabolic pathway is dysregulated in a sMN ALS cell, including: isolating the sMN ALS cell: isolating an oMN ALS cell: isolating total RNA from the sMN cell: isolating total RNA from the oMN cell: and performing a differential gene expression assay from the total RNA from the sMN cell and from the total RNA from the oMN cell, the differential gene expression assay including comparing an expression level of a gene associated with the metabolic pathway from the sMN ALS cell with an expression level of the gene associated with the metabolic pathway from the oMN ALS cell: where a difference in the expression level of the gene associated with the metabolic pathway from the sMN ALS cell as compared to the expression level of the gene associated with the metabolic pathway from the oMN ALS cell is indicative of a dysregulation of the metabolic pathway.

### BRIEF DESCRIPTION OF THE DRAWINGS

FIGS. 1A-1K are images and data graphs showing the differentiation of PHOX2B::GFP⁺ and HB9::GFP⁺ neurons according to an embodiment.

FIGS. 2A-2E show an illustration and data graphs showing that genome-wide RNA sequencing analysis reveals aberrant lipid metabolism after comparison between post-sorted HB9::GFP⁺ and PHOX2B::GFP⁺ in SODIAH and C9ORF72 ALS lines according to an embodiment.

FIGS. 3A-3D show an illustration and data graphs showing metabolomics analysis indicates up-regulation of lipid metabolism in post sorted HB9::GFP⁺ of SOD1^{A4V} and C9ORF72 ALS lines according to an embodiment.

FIGS. 4A-4E are data graphs showing metabolomics analysis in un-sorted sMN differentiation confirmed up-regulation of lipid metabolism, and provides lipid related metabolic candidates in TDP43^{Q343R}, C9ORF72, SOD1^{A4V} and Sporadic ALS lines according to an embodiment.

FIGS. 5A-5E are data graphs showing that 5-LOX inhibitors rescue motor neuron degeneration in vitro according to an embodiment.

FIGS. 6A-6L are images and data graphs showing that 5-LOX inhibitors rescue the phenotype of Drosophila model and aberrant AA pathways in vitro according to an embodiment.

FIGS. 7A-7L are images and data graphs showing the characterization of transcripts in hiPSC derived PHOX2B::GFP⁺ OMN-like cells according to an embodiment.

FIGS. 8A-8G show an illustration and data graphs showing that transcriptome profiling reveals differences between PHOX2B::GFP⁺ and HB9::GFP⁺ cells in both SOD1^{A4V} and C9ORF72 ALS lines according to an embodiment.

FIGS. 9A-9K are data graphs showing selection of altered metabolic candidates by metabolomics analysis in ALS lines according to an embodiment.

FIGS. 10A-10H are data graphs showing that caffeic acid exclusively rescues HB9::GFP⁺ cells in SOD1^{A4V} and C9ORF72 according to an embodiment.

FIGS. 11A and 11B are lists of the top ranked perturbed pathways according to an embodiment

FIGS. 12A-12H are data graphs and images showing that caffein acid alleviates disease pathogenesis in SOD1^{G934} mice.

FIGS. 13A-13K are karyotypes, images and data graphs showing the generation of PHOX2B::GFP reporter line and oMN-like cell specification in SOD1^{A4V} and C9ORF72 ALS lines according to one embodiment.

FIGS. 14A-14H are data graphs and heat maps showing oMN-like cell maturation in control, SOD1^{A4V} and C9ORF72 lines according to one embodiment.

FIGS. 15A-15I are images, karyotypes, a construction schematic, data graph and heat maps showing how HB9::GFP reporter in SOD1^{A4V} and C9ORF72 ALS lines was generated according to one embodiment.

FIGS. 16A-16I are data graphs and FACS dot displays showing the expression of sMN specific markers in SOD1^{A4V} and C9ORF72 derived HB9::GFP⁺ cells according to an embodiment.

FIGS. 17A-17G are graphs showing the characterization of sMN subtypes by maker expression in different differentiation time of C9ROF72 and SOD1^{A4V} ALS hiPSC lines according to an embodiment.

FIGS. 18A-18B are heat maps showing the validation of oMN and sMN population by comparing transcriptome profile with reference dataset according to an embodiment.

FIGS. 19A-19G are a schematic, heat maps and metabolomics analysis comparing transcriptome profiles of healthy hESC and hiPSC-derived PHOX2B::GFP⁺ cells and HB9 antibody-stained cells according to an embodiment.

FIGS. 20A-20J are heat maps and data graphs showing abnormal expression of lipid related transcripts in SOD1^{A4V} and C9ORF72 ALS lines by qRT-PCR analysis according to an embodiment.

FIGS. 21A-21D are data graphs and heat maps showing common alteration of C21H26O3 in multiple ALS lines and direct comparison of altered metabolic metabolites by metabolomics analysis in isogenic control of SOD1^{A4V} and SOD1^{A4V} ALS hiPSC lines according to an embodiment.

FIGS. 22A-22H are images, data graphs, and schematics showing that caffeic acid alleviates disease pathogenesis in SOD1^{G934} mice according to an embodiment.

FIGS. 23A-23G are a schematic model of the study and data graphs showing that caffeic acid rescues aberrant levels of arachidonic acid in the sMN culture of multiple ALS hiPC lines according to an embodiment.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

An embodiment of the invention relates to a method of treating an amyotrophic lateral sclerosis (ALS) cell, including: selecting a therapeutic compound; treating an aberrant arachidonic acid (AA) metabolic pathway in the ALS cell including contacting the ALS cell with the therapeutic compound.

An embodiment of the invention relates to the method above, where the treating the aberrant arachidonic acid (AA) metabolic pathway results in a reduction of a cellular level of AA in the ALS cell.

An embodiment of the invention relates to the method above, where the therapeutic compound is an inhibitor of 5-lipoxygenase (5-LOX).

An embodiment of the invention relates to the method above, where the inhibitor of 5-LOX includes a redox-active compound, an iron ligand inhibitor, a non-redox-type inhibitor, a redox-type inhibitor, a Dual (COX/5-LOX) type inhibitor, or an iron ligand-type inhibitor.

An embodiment of the invention relates to the method above, where the inhibitor of 5-LOX includes a redox-active inhibitor.

An embodiment of the invention relates to the method above, where the inhibitor of 5-LOX includes caffeic acid (3,4-dihydroxy benenearcrylic acid), apigenin, BW755C, nordihydroguaretic acid, or a functional analog or derivative thereof.

An embodiment of the invention relates to a method of treating a subject with ALS, including: selecting a therapeutic compound: and treating an aberrant arachidonic acid (AA) metabolic pathway in the subject including administering to the subject the therapeutic compound.

An embodiment of the invention relates to the method above, where the therapeutic compound results in a reduction of a cellular level of AA in the spinal motor neuron cell of the subject.

An embodiment of the invention relates to the method above, where the therapeutic compound is an inhibitor of 5-lipoxygenase (5-LOX).

An embodiment of the invention relates to the method above, where the inhibitor of 5-LOX includes a redox-active compound, an iron ligand inhibitor, a non-redox-type inhibitor, a redox-type inhibitor, a Dual (COX/5-LOX) type inhibitor, or an iron ligand-type inhibitor.

An embodiment of the invention relates to the method above, where the inhibitor of 5-LOX includes a redox-active inhibitor.

As used throughout, the terms “5-LOX inhibitor” and “inhibitor of 5-LOX” are used interchangeably throughout. In general, the four classes of direct 5-lipoxygenase inhibitors encompass: i) redox-active compounds that interrupt the redox cycle of the enzyme, ii) iron ligand inhibitors that chelate the active site iron, iii) nonredox-type inhibitors that compete with arachidonic acid and iv) novel class inhibitors that may act in an allosteric manner.

According to some embodiments, redox-active 5-LOX inhibitors comprise lipophilic reducing agents including many natural plant-derived (e.g., nordihydroguaretic acid, caffeic acid, flavonoids, coumarins and several polyphenols) and synthetic compounds. The first synthetic 5-LOX inhibitors such as AA-861, L-656,224, phenidone or BW755C belong to this class. These drugs act by keeping the active site iron in the ferrous state, thereby, uncoupling the catalytic cycle of the enzyme.

According to some embodiments, iron ligand inhibitors represent hydroxamic acids or N-hydroxyurea derivatives that chelate the active site iron but also possess weak reducing properties. The hydroxamic acid BWA4C and the hydrolytic-stable N-hydroxyurea derivative zileuton are potent and orally active 5-LOX inhibitors. Some examples include Zileuton, ABT-761, and LDP-977 (CMI-977).

According to some embodiments, nonredox-type 5-LOX inhibitors compete with AA or LOOH for binding to 5-LOX. They are devoid of redox properties and encompass structurally diverse molecules. Representatives out of this class such as the orally active compounds ZD 2138, L-739,010 or CJ-13,610 as well as the thiopyranoindole L-699,333 are highly potent and selective for 5-LOX in cellular assays, with IC50 values in the low nanomolar range.

Some embodiments relate to the use of a 5-LOX inhibitor which binds to other relevant targets including COX enzymes, the PAF or the HI receptor (so-called dual inhibitors). One example of such a dual 5-LOX/COX pathway inhibitors includes licofelone.

According to some embodiments, 5-LOX inhibitors may include the polyphenolic (b)-3,4,3′,4′-tetrahydroxy-9,7′a-epoxy lignano-7 a,9-lactone, novel caffeoyl clusters (trimers or tetramers), NSAIDs that are covalently linked to an iron-chelating moiety, the urea derivative RBx 7796, substituted coumarins based on the structure of L-739,010, fluorophenyl-substituted coumarins where the thioaryl moiety carrying the hexafluorcarbinol is replaced by an amino-oxadiazol moiety, tetrahydropyrane-carboxamides (exemplified by CJ-13,610), tricyclic thiazole-based derivatives with a thiazolone core moiety, tetrahydronaphtol derivatives, sulfonamide-spiro(2H-1-benzopyrane-2,4-piperidin) derivatives, benzoxazole derivatives, Licofelone, macrolide conjugates, oflicofelone or related pyrrolizine and indolizine derivatives with macrocyclic antibiotics, celecoxib, etoricoxib, rofecoxib, novel diaryl-dithiolanes and isothiazoles, rofecoxib derivatives, 1,3-diarylprop-2-yn-ones with a C3 p-SO2Me COX-2 pharmacophore, 7-tert-butyl-2,3-dihydro-3,3-dimethylbenzofuran derivatives, phenylsulfonyl urenyl chalcone derivatives, 2-(4-aminophenyl)-3-(3,5-dihydroxylphenyl) propenoic acid, a-(n)-alkyl-substituted pir-inixic acid derivatives, indole-3-carboxylates, 2-amino-5-hydroxyindole-3-carboxylates, benzo[g]indole-3-carbox-ylates, sulfonimides based on the aryl-pyrrolizine scaffold oflicofelone, lipophilic phenolic compounds such as curcumin from turmeric, garcinol from the fruit rind of Guttiferae species, myrtucommulone from myrtle (Myrtus communis) and epigallocatechin-3-gallate from green tea (Camellia sinensis), hyperforin. Examples of 5-LOX inhibitors are not restricted to the list above, or to specific compounds disclosed herein.

An embodiment of the invention relates to a method of differentiating a human stem cell to an ocular motor neuron (oMN) ALS-specific human cell type, including: culturing the human stem cell in a first media including recombinant sonic hedgehog signaling protein and purmorphamine for 9 days: culturing the human stem cell in a second media including brain-derived neurotrophic factor (BDNF), glial cell line-derived neurotrophic factor (GDNF), and Ascorbic Acid for at least 1 day. In such an embodiment, the second media does not include sonic hedgehog signaling protein or purmorphamine.

An embodiment of the invention relates to the method above, where the human stem cell is an embryonic human stem cell or a human induced pluripotent stem cell.

An embodiment of the invention relates to the method above, where an expression of at least one oMN-specific gene is increased in the oMN ALS-specific human cell.

An embodiment of the invention relates to the method above, where the at least one oMN-specific gene is selected from the list consisting of ISL1, PHOX2A, NKX6.1, EN1, CHAT, PHOX2B, TBX20, FGF10, EYA1, EYA2, PLEXINA4, SEMA6D and MAP2.

An embodiment of the invention relates to a method of differentiating a human stem cell to an ocular motor neuron (oMN) ALS-specific human cell type, including: culturing the human stem cell in a first media including recombinant sonic hedgehog signaling protein and purmorphamine for 9 days: culturing the human stem cell in a second media including brain-derived neurotrophic factor (BDNF), glial cell line-derived neurotrophic factor (GDNF), and Ascorbic Acid for at least 1 day. In such an embodiment, the second media does not include sonic hedgehog signaling protein or purmorphamine. Table 1 discloses example media types, compounds, and incubation periods of such a method.

TABLE 1
Example protocol for differentiating a human stem cell to
an ocular motor neuron (oMN) ALS-specific human cell type.

| Day | Media | Compounds (concentration) |
|---|---|---|
| Day 0 | KSR | LDN193189(500 nM)/SB431542 (10 μM) |
| Day 1 | KSR | LDN193189(500 nM)/SB431542(10 μM)/ |
| | | SHH(50 ng/ml)/Purmorphamine(1 μM)/ |
| | | FGF8(100 ng/ml) |
| Day 3 | KSR | LDN193189(500 nM)/SB431542 (10 μM)/ |
| | | SHH(50 ng/ml)/Purmorphamine(1 μM)/ |
| | | FGF8(100 ng/ml)/CHIR99021(3 μM) |
| Day 5 | KSR 75% + | LDN193189(500 nM)/SB431542 (10 μM)/ |
| | NB 25% | SHH(50 ng/ml)/Purmorphamine(1 μM)/ |
| | | FGF8(100 ng/ml)/CHIR99021(3 μM) |
| Day 7 | KSR 50% + | LDN193189(500 nM)/SHH(50 ng/ml)/ |
| | NB 50% | Purmorphamine(1 μM)/FGF8(100 ng/ml)/ |
| | | CHIR99021(3 μM) |
| Day 9 | KSR 25% + | LDN193189(500 nM)/SHH(50 ng/ml)/ |
| | NB 75% | Purmorphamine(1 μM)/FGF8(100 ng/ml)/ |
| | | CHIR99021(3 μM) |
| Day 11 | NB | CHIR99021(3 μM)/BDNF(20 ng/ml)/ |
| | | GDNF(10 ng/ml)/Ascorbic acid(200 μM)/ |
| | | dcAMP(200 μM)/DAPT(10 μM) |
| Day 13 | NB | BDNF(20 ng/ml)/GDNF(10 ng/ml)/ |
| to harvest | | Ascorbic acid(200 μM)/dcAMP(200 μM)/ |
| | | DAPT(10 μM) |

An embodiment of the invention relates to an ocular motor neuron (oMN) ALS-specific human cell generated from the methods discussed above.

An embodiment of the invention relates to a method for identifying whether a metabolic pathway is dysregulated in a sMN ALS cell, including: isolating the sMN ALS cell; isolating an oMN ALS cell; isolating total RNA from the sMN cell; isolating total RNA from the oMN cell; and performing a differential gene expression assay from the total RNA from the sMN cell and from the total RNA from the oMN cell, the differential gene expression assay including comparing an expression level of a gene associated with the metabolic pathway from the sMN ALS cell with an expression level of the gene associated with the metabolic pathway from the oMN ALS cell: where a difference in the expression level of the gene associated with the metabolic pathway from the sMN ALS cell as compared to the expression level of the gene associated with the metabolic pathway from the oMN ALS cell is indicative of a dysregulation of the metabolic pathway.

An embodiment of the invention relates to the method above, where the sMN ALS cell is differentiated from a human stem cell.

An embodiment of the invention relates to the method above, where the oMN ALS cell is differentiated from a human stem cell.

An embodiment of the invention relates to the method above, further including determining whether a metabolite associated with the metabolic pathway is dysregulated in the sMN ALS cell, including: isolating the metabolite from the sMN cell: isolating the metabolite from the oMN cell: determining the relative abundance of the metabolite from the sMN cell: determining the relative abundance of the metabolite from the oMN cell; and comparing the relative abundance of the metabolite from the sMN cell with the relative abundance of the metabolite from the oMN cell. In such an embodiment, a difference in the relative abundance of the metabolite from the sMN cell as compared to the relative abundance of the metabolite from the oMN cell indicative of a dysregulation of the metabolic pathway.

The following describes some embodiments of the current invention more specifically. The general concepts of this invention are not limited to these particular embodiments.

### Example

In the following example, data from studies showing the identification of cell intrinsic factor(s) that cause differential susceptibility between sMN and oMN subtypes in ALS are presented. Transcriptomics and metabolomics profiling was used to generate the data. The results reveal aberrant lipid metabolism in ALS patient-derived sMN populations. One of major dysregulated metabolism pathways is the off-controlled arachidonic acid (AA) metabolism, whose pharmacological modulation unexpectedly increased the survival rates of HB9::GFP⁺ ALS sMNs and partially reversed ALS-related phenotypes in a Drosophila and SOD1^{G944} mouse model. These studies provide new insights into ALS pathogenic mechanism and potential therapeutic targets for both fALS and sALS.

### RESULTS

PHOX2B genetic reporter system enriched hESC and iPSC-derived oMN-like molecular patterns

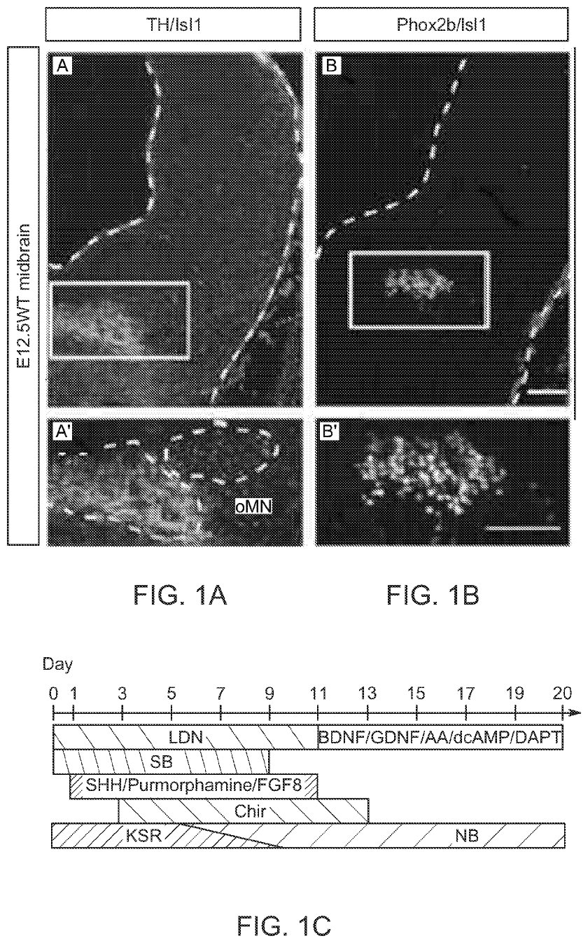

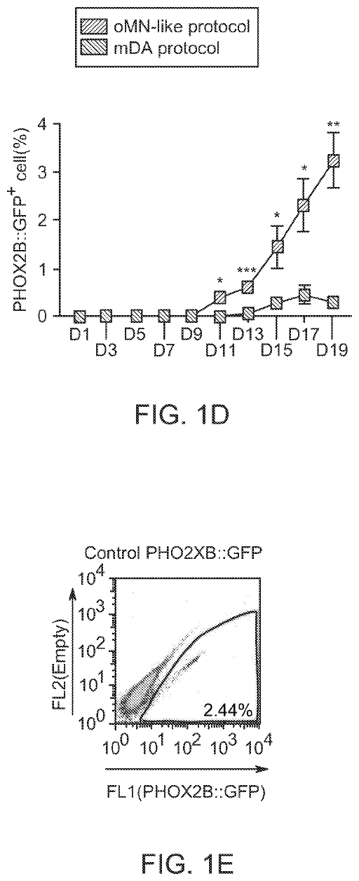

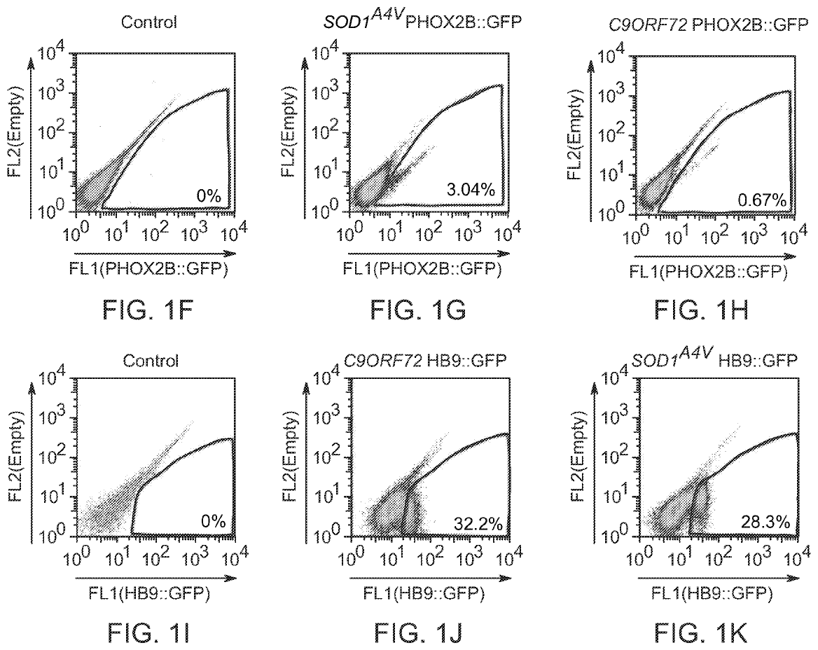

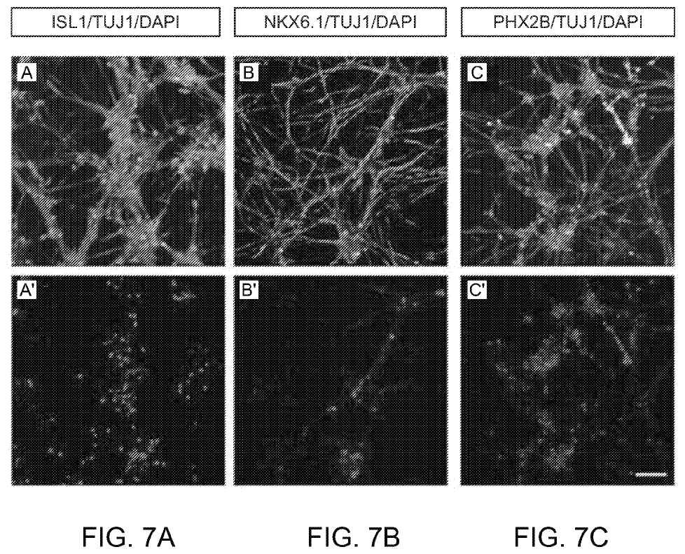

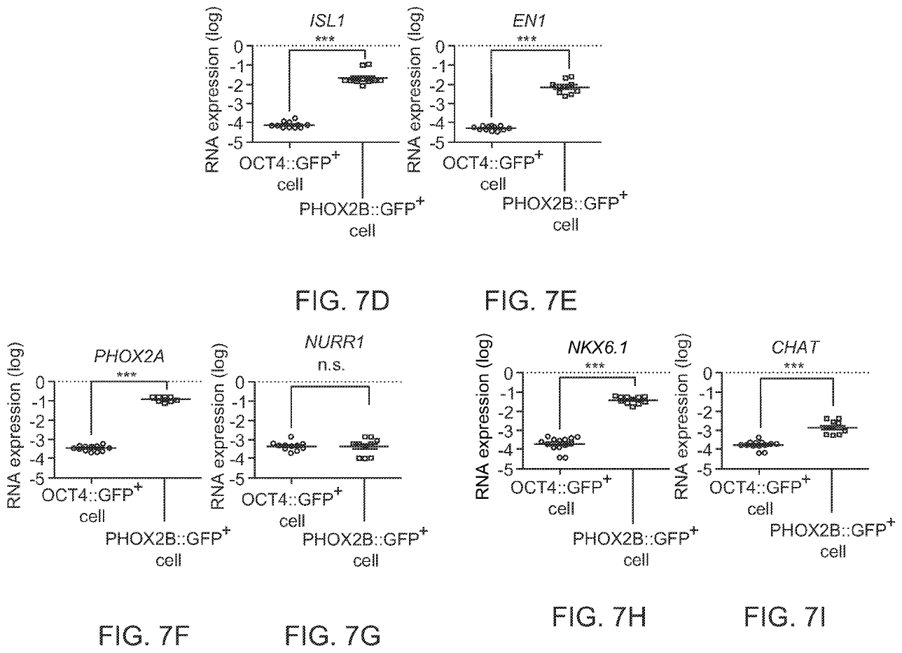

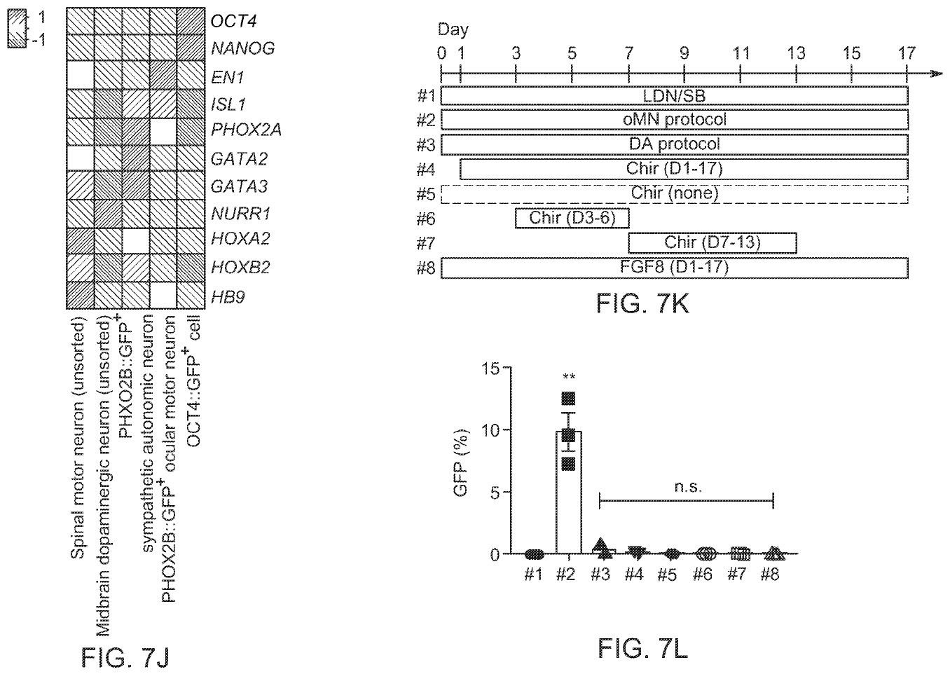

The transcriptional remodeling of oMN during development is different from that of sMNs in the spinal cord⁴⁷. Initially, Nkx6.1⁺ basal plate cells in the midbrain give rise to oMN precursors and differentiated Phox2a/b-expressing oMNs to form the ocular motor nucleus in the midbrain ventrolateral region⁴⁸⁻⁵¹. First, expression of several markers for oMNs in embryonic mouse midbrains were evaluated by immunohistochemistry (FIGS. 1A-B′). Isl1 has been shown to be a key transcription factor in regulating oMN-specification in the developing midbrain⁴⁹, and the expression pattern of Phox2b, a homeodomain transcription factor, overlaps with that of Isl1 (FIGS. 1B-B′). Previous studies using mouse genetics have demonstrated that proper expression of Phox2b is required for brachial motor neuron development, but not for somatic motor neurons including sMNs in the central nervous system (CNS)^{47,52}. Therefore, mutations in phox2a/b have been shown to be specifically relevant to ocular motor genetic disorders⁵³⁻⁵⁵. Based on these findings, PHOX2B::GFP reporter human embryonic stem cells (hESC)s and hiPSCs were generated using the CRISPR-Cas9 system⁵⁶. This reporter system has allowed the development of an oMN-like cell differentiation protocol by modification of midbrain dopaminergic neuronal (mDA) differentiation methodology⁵⁷. In the developing mouse brain, it was observed that the TH⁺ mDA neurons were located in the ventral region of midbrain, but distinctly separate from oMNs (FIG. 1A′). During neurogenesis of the ventral region of the midbrain, the sonic hedgehog (SHH) signaling pathway is one of the key regulators of oMNs specification⁵⁸. Therefore, the dosage of recombinant SHH protein/purmorphamine (PMP) treatment was modified in the mDA differentiation method (FIG. 1C). The new protocol significantly increased the efficiency of obtaining PHOX2B::GFP⁺ cell differentiation compared to the mDA method (FIGS. 1D-E and FIGS. 7K-L). Post-purified PHOX2B::GFP⁺ cells showed enriched marker protein expression including ISL1, NKX6.1 and PHOX2B (FIGS. 7A-7C′), suggesting that the new protocol provides selective cell lineage of oMN-like hESC and hiPSC. qRT-PCR analysis also confirmed this by showing the enrichment of transcripts (ISL1, PHOX2A, NKX6.1, EN1 and CHAT) expressed in oMN-like cells selected by the protocol, but not in mDA enriched cells (NURR1) (FIGS. 7D-7I). Finally, limited inclusion of peripheral autonomic neurons was confirmed by profiling genetic expression including EN1, a regional marker of midbrain^{56,59} and GATA2 and 3, specific marker for peripheral autonomic neurons⁶⁰ (FIG. 7J).

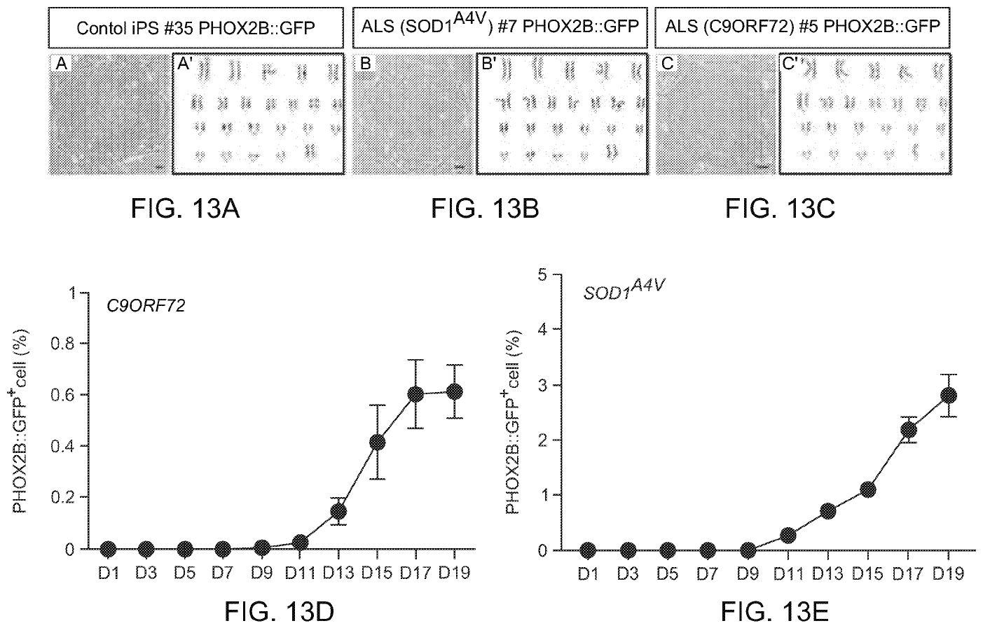

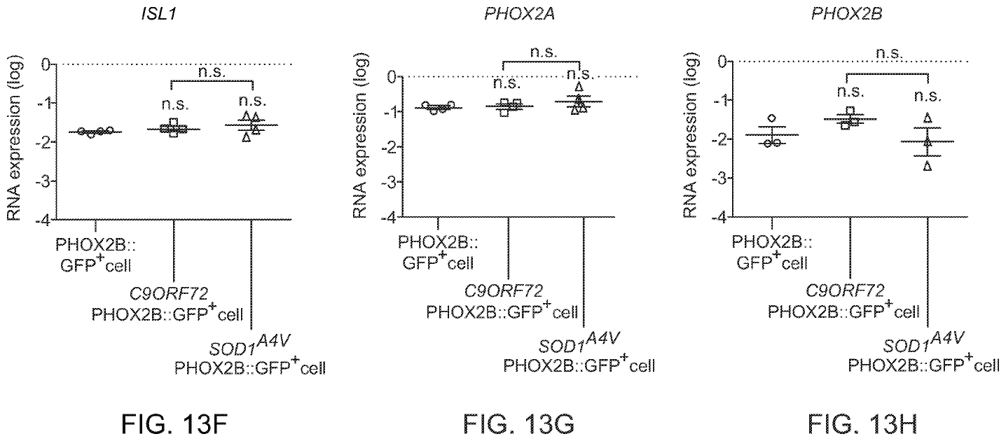

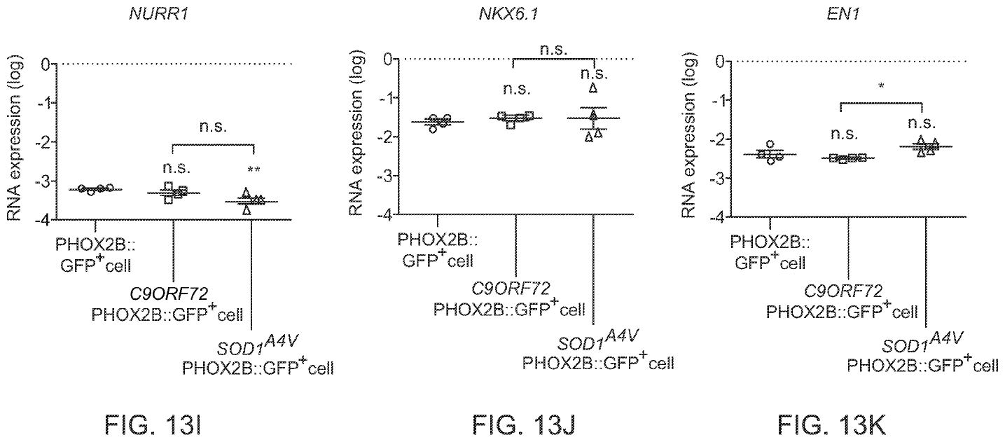

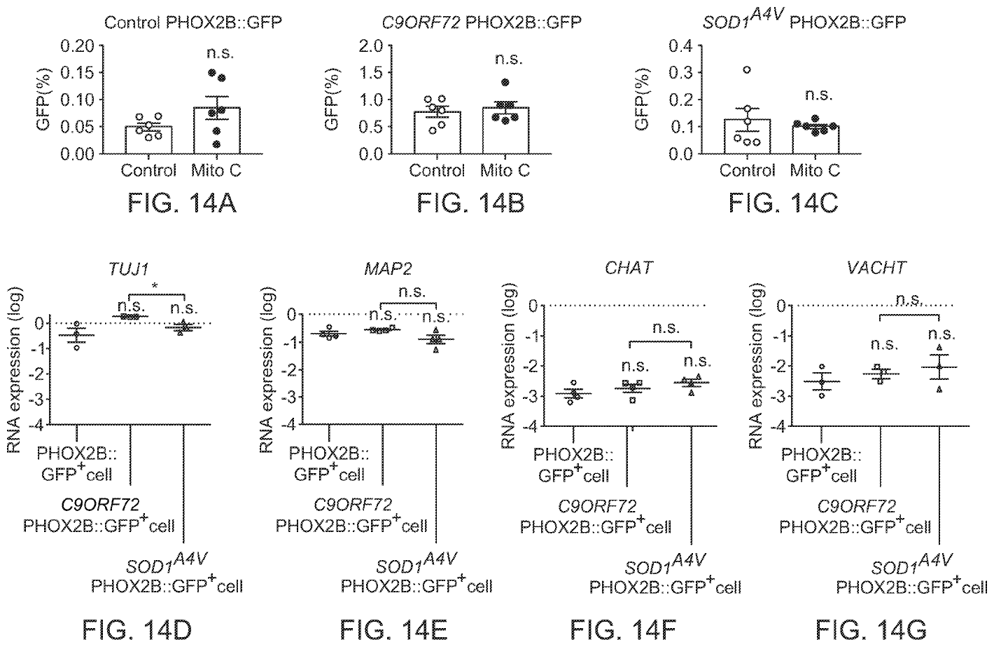

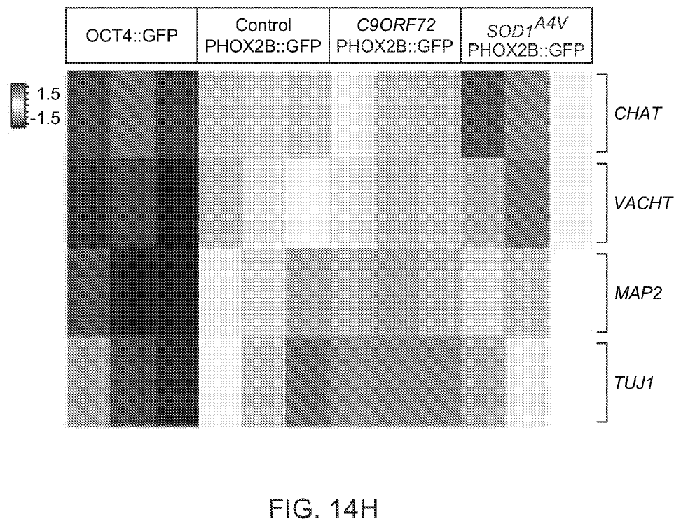

To isolate pure oMN-like cells from ALS hiPSC lines, a PHOX2B::GFP reporter from healthy control and ALS hiPSC lines (SOD1^{A4V} and (9ORF72 with 500 GGGGCC hexanucleotide repeats SEQ ID NO: 81) was generated using the CRISPR-Cas9 system (FIG. 1F-H and FIGS. 13A-C′). It was determined that PHOX2B::GFP expression patterns in the hiPSC lines were similar to that of wild type hESCs (FIG. 1D and FIGS. 13D-E). qRT-PCR analysis also revealed that oMN-specific marker genes (ISL1, PHOX2A, NKX6.1, EN1, CHAT and MAP2) were enriched, but not NURR1 transcript (mDA marker) in the post-sorted PHOX2B::GFP⁺ cells of ALS lines as seen in PHOX2B::GFP⁺ cells (FIGS. 13F-K). qRT-PCR analysis also provided highly enriched neuronal maturation makers (TUJ1, MAP2, CHAT and VACHT) in post-sorted oMN-like cells (FIGS. 14A-14H). Taken together, these data confirm that PHOX2B::GFP⁺ cells derived from healthy and ALS hESCs/hiPSCs commonly showed oMN-like profiles.

FIGS. 1A-1I are images and data graphs showing the differentiation of PHOX2B::GFP⁺ and HB9::GFP⁺ neurons according to an embodiment. More specifically, FIGS. 1A-1B′ show identification of neuronal subtypes in mouse midbrain using Isl1 and Phox2b for oMN, and TH for mDA. (FIG. 1C) Schematic protocol of oMN-like cell differentiation. (FIG. 1D) Time course comparison of PHOX2B::GFP⁺ expression between oMN-like and mDA protocol by FACS (oMN-like: n=10, mDA: n=5, *P<0.05, **P<0.01, ***P<0.001, unpaired student's t-test). (FIGS. 1E-1I) Representative FACS plot of PHOX2B::GFP reporter line for oMN-like cell differentiation in control, SOD1^{A4V} and C90RF72 lines (FIGS. 1E-1G) and HB9::GFP reporter line for sMN differentiation in SOD1^{A4V} and C9ORF72 lines (FIGS. 1H-1I). Scale bars, 100 μm. Error bars, mean±SEM. oMN, ocular motor neuron. sMN, spinal motor neuron. mDA, midbrain dopaminergic neuron. TH, tyrosine hydroxylase.

FIGS. 7A-7L are images and data graphs showing the characterization of transcripts in hiPSC derived PHOX2B::GFP⁺ oMN-like cells according to an embodiment. (FIGS. 7A-7C′) Characterization of post-sorted PHOX2B::GFP⁺ cells using ISL1, NKX6.1 and PHOX2B (red), and TUJ1 (green) antibodies. (FIGS. 7D-7I) qRT-PCR analysis shows enrichments of oMN specification transcripts (ISL1, PHOX2A, NKX6.1 and CHAT) and midbrain regional transcript (EN1), but not mDA specification transcript (NURR1) after sorting (D14) (n=4 for each group, n.s.: not significant, ***P<0.001, unpaired student's t-test). (FIG. 7J) Heatmap presents characteristic marker expression of ES (OCT4::GFP), oMN-like (PHOX2B::GFP), sympathetic autonomic neuron (PHOX2B::GFP), mDA (unsorting) and sMN (unsorting) (n=3 for each group: technical replicates). (FIGS. 7K-L) Different schematic protocols to optimize oMN-like cell differentiation (FIG. 7K) and FACS results of PHOX2B::GFP⁺ (FIG. 7L) (n=3 for each groups: technical replicates: n.s.: not significant, **P<0.01, unpaired student's t-test). Error bars, mean±SEM., PHX2B: PHOX2B.

FIGS. 13A-K are karyotypes and data graphs showing the generation of PHOX2B::GFP reporter line and oMN-like cell specification in SOD1^{A4V} and C9ORF72 ALS lines according to one embodiment. (FIGS. 13A-C′) Representative images and karyotype results of control hiPSC, SOD1^{A4V} and C9ORF72 PHOX2B::GFP reporter lines. (FIGS. 13D-E) Time course GFP expression of oMN-like differentiation in SOD1^{A4V} and C9ORF72 by FACS analysis (SOD1^{A4V}: n=3, C9ORF72: n=4). (FIGS. 13F-L) Enrichment of transcripts in post-sorted ES derived and both ALS derived PHOX2B::GFP⁺ is comparable for oMN (ISL1, PHOX2A/B and NKX6.1), midbrain specification (EN1) and mDA specification (NURR1) by qRT-PCR analysis (D14) (at least n=4 for each group: technical replicates: n.s.: not significant, *, p<0.05: unpaired student's t-test). Error bars, mean±SEM.

FIGS. 14A-14H are data graphs and heat maps showing oMN-like cell maturation in control, SOD1^{A4V} and C9ORF72 lines according to one embodiment. (FIGS. 14A-C) FACS results of PHOX2B::GFP⁺ between control (non-treated) and mitomycin C treated (1 μg/ml, 1 hr: D17 to D19 (2 days)) (n=6 for each group: technical replicates: n.s.: not significant: unpaired student's t-test). (FIGS. 14D-H) Enrichment of transcripts (TUJ1, MAP 2, CHAT and VACHT) in post-sorted control, C9ORF72 and SOD1^{A4V} lines for maturation of oMN-like cells by qRT-PCR (FIGS. 14D-G) and heatmap for comparison with ES (OCT4::GFP) (H) (n=3 for each groups: technical replicates: n.s.: not significant: *, p<0.05; unpaired student's t-test). Error bars: mean±SEM.

HB9::GFP neurons represent ALS hiPSC-derived sMN cells

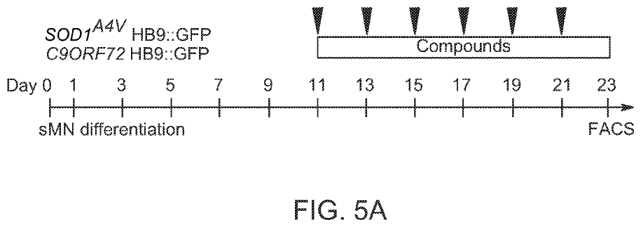

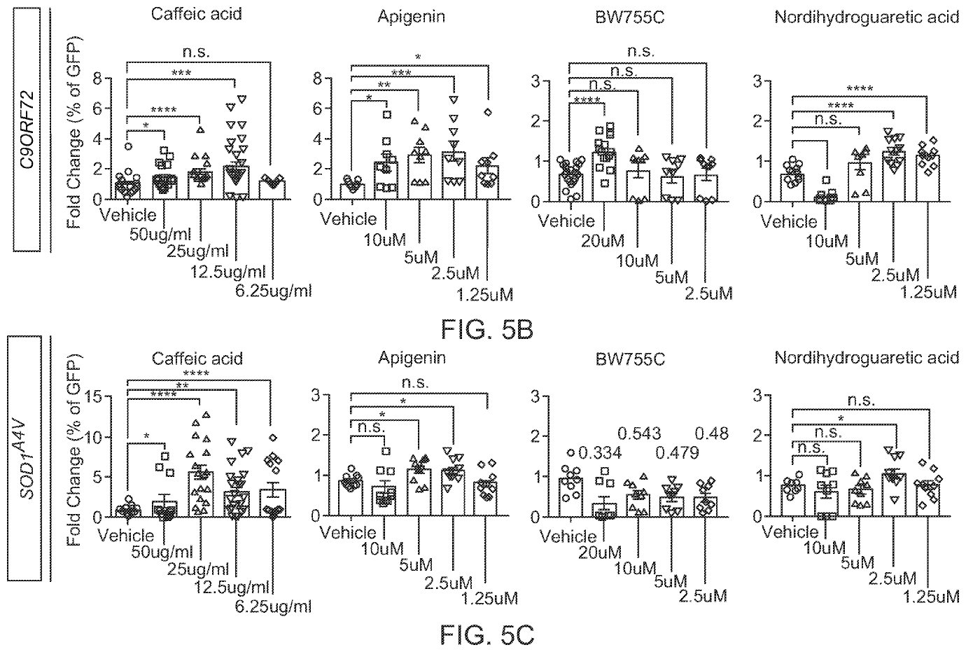

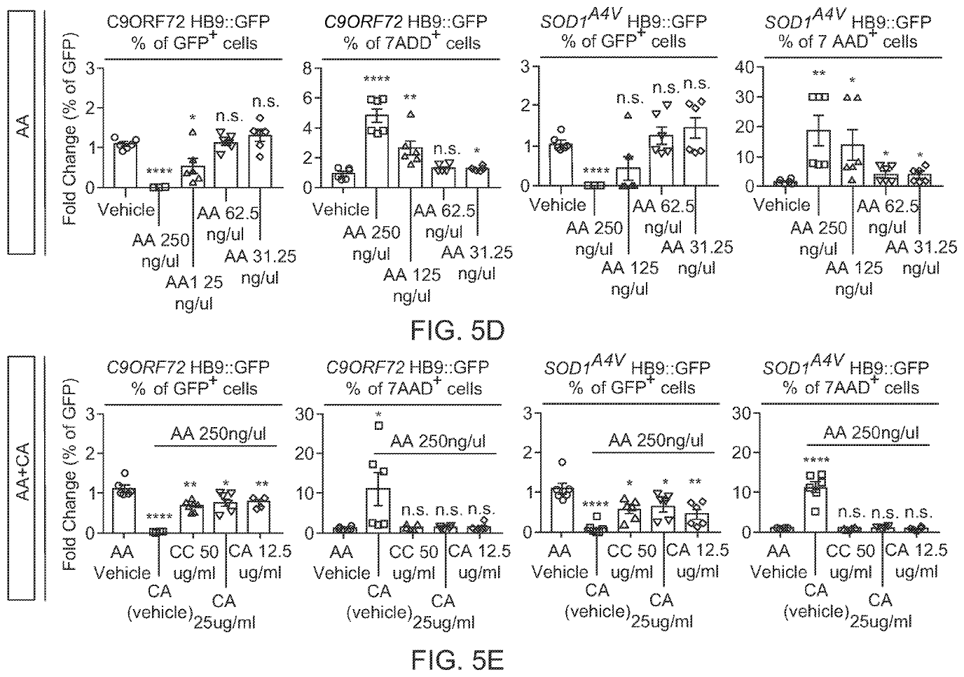

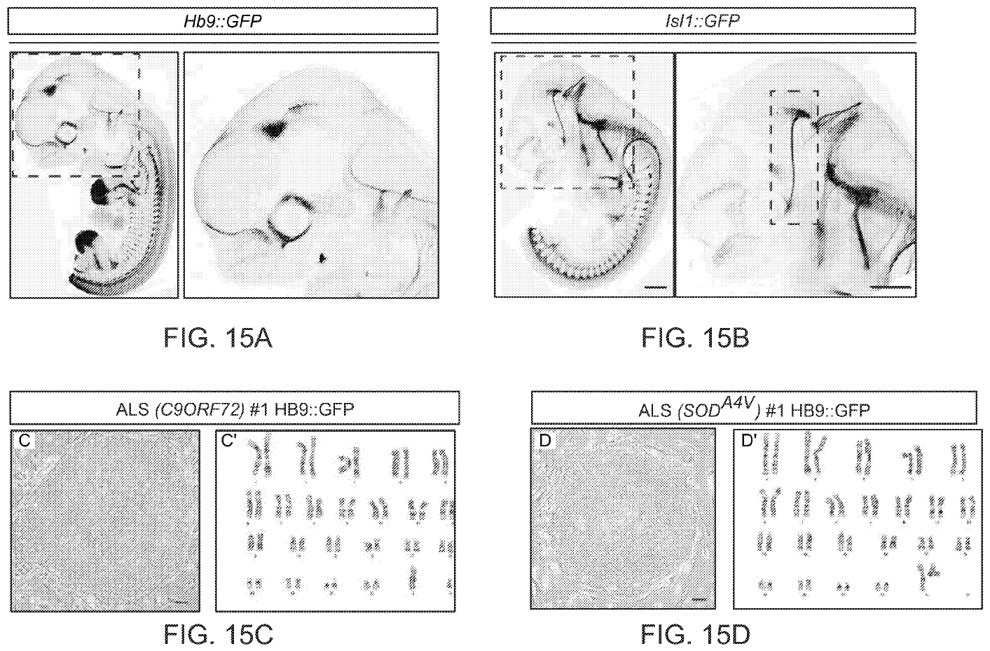

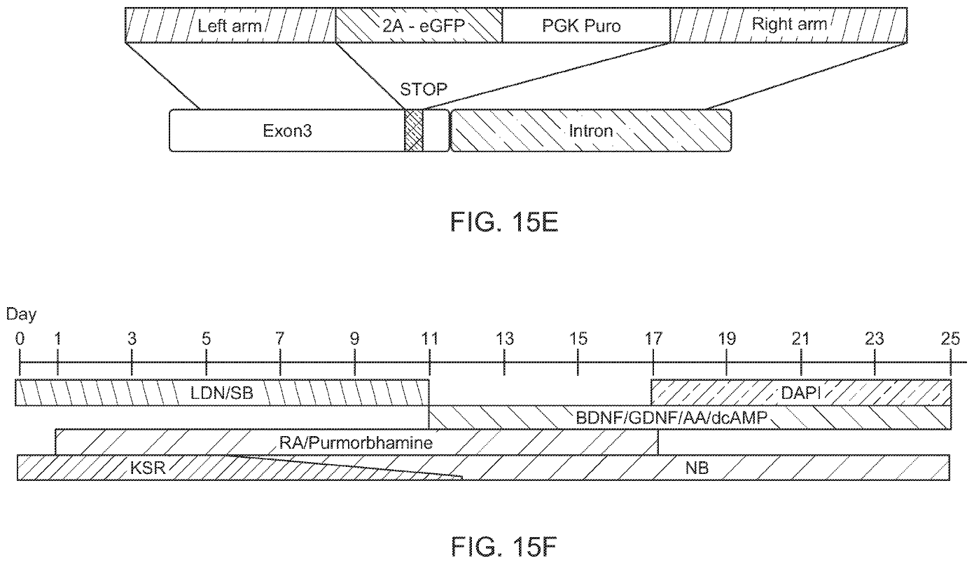

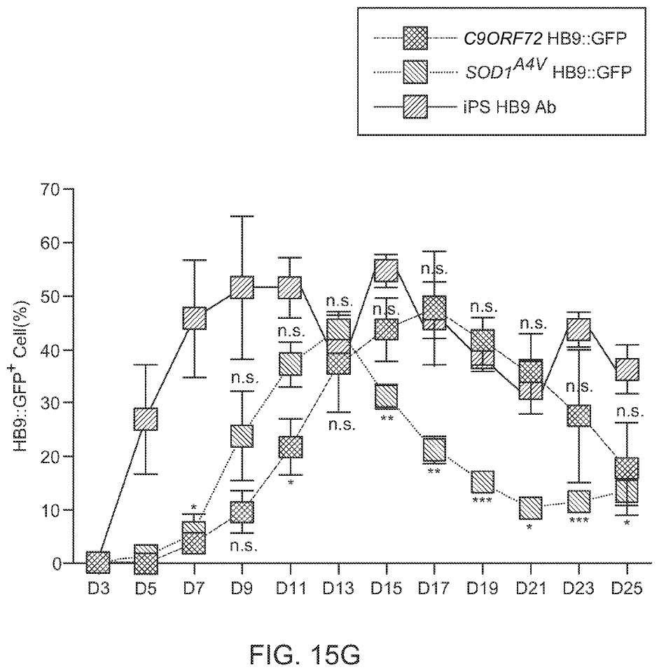

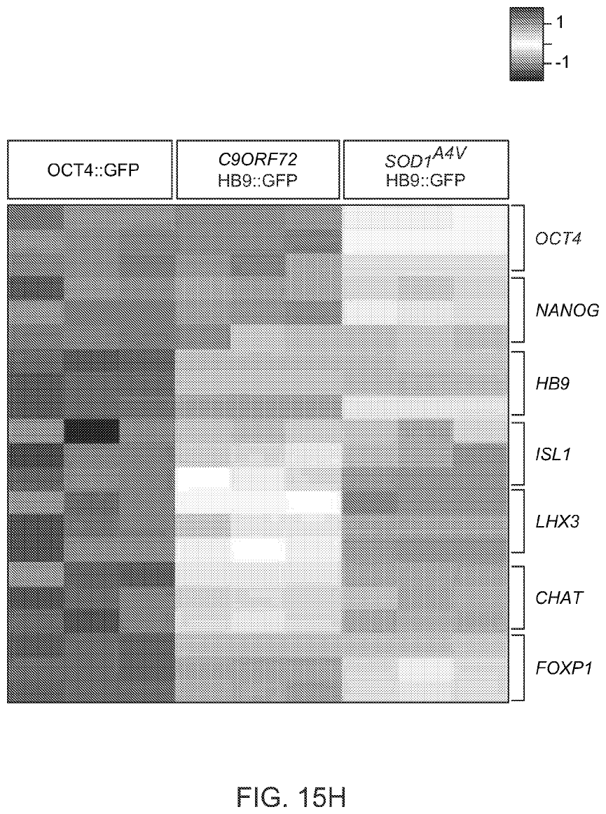

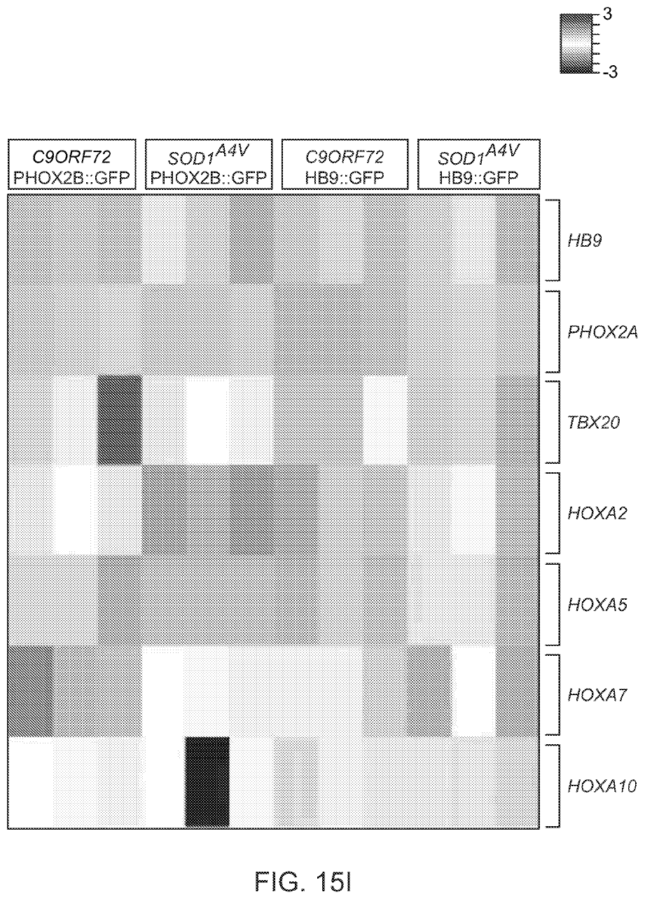

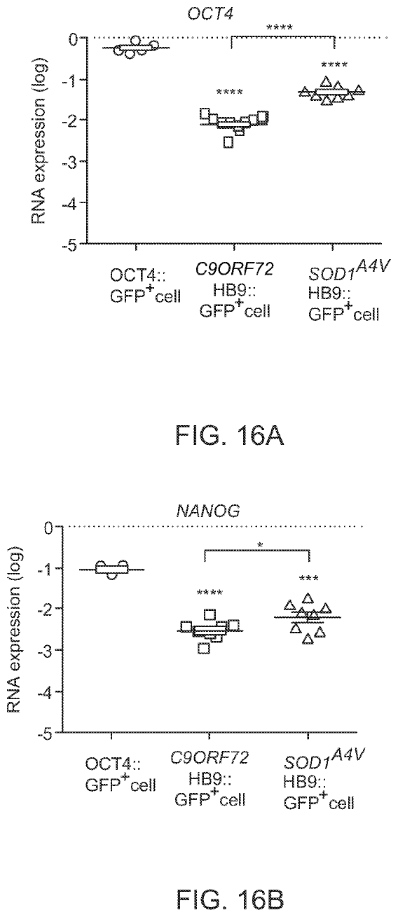

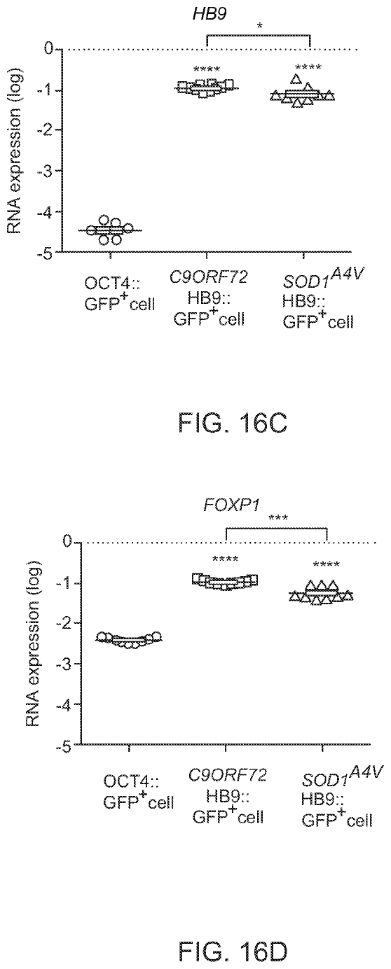

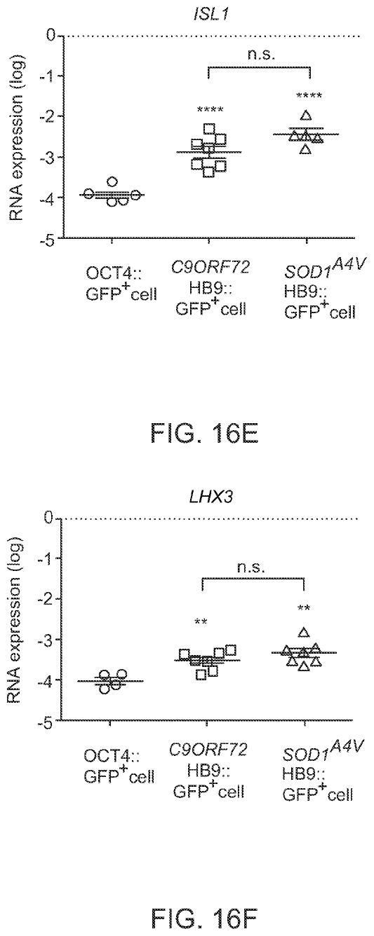

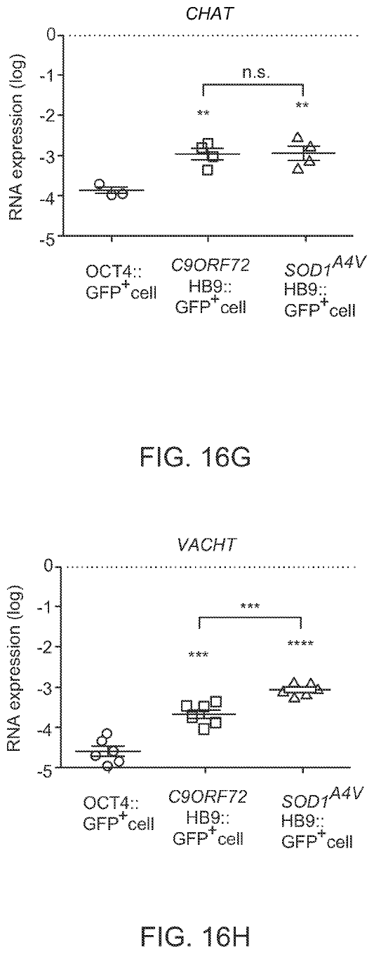

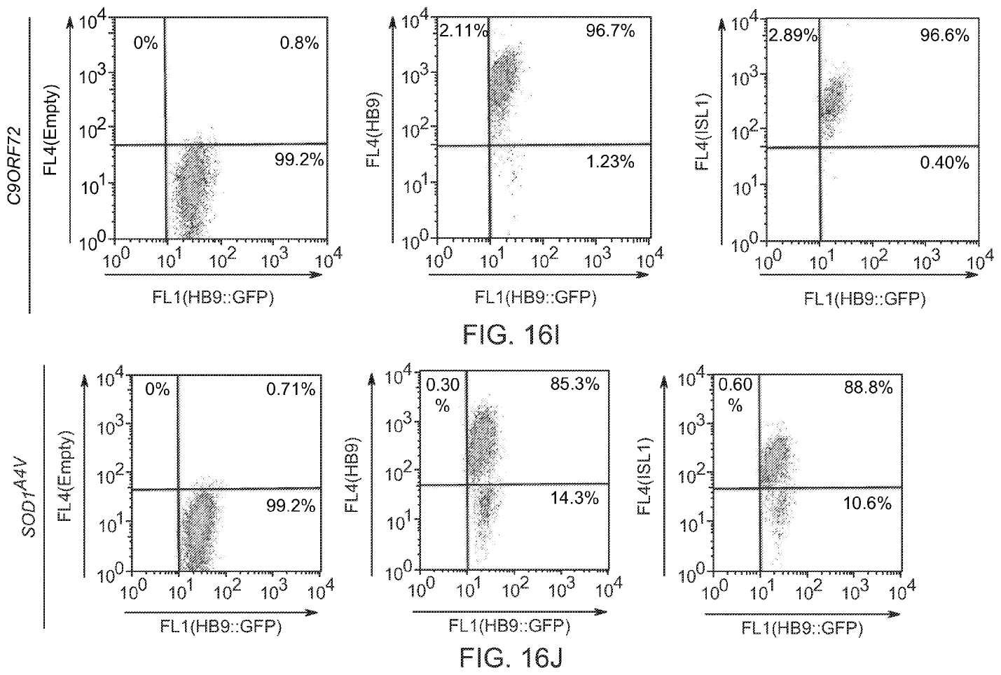

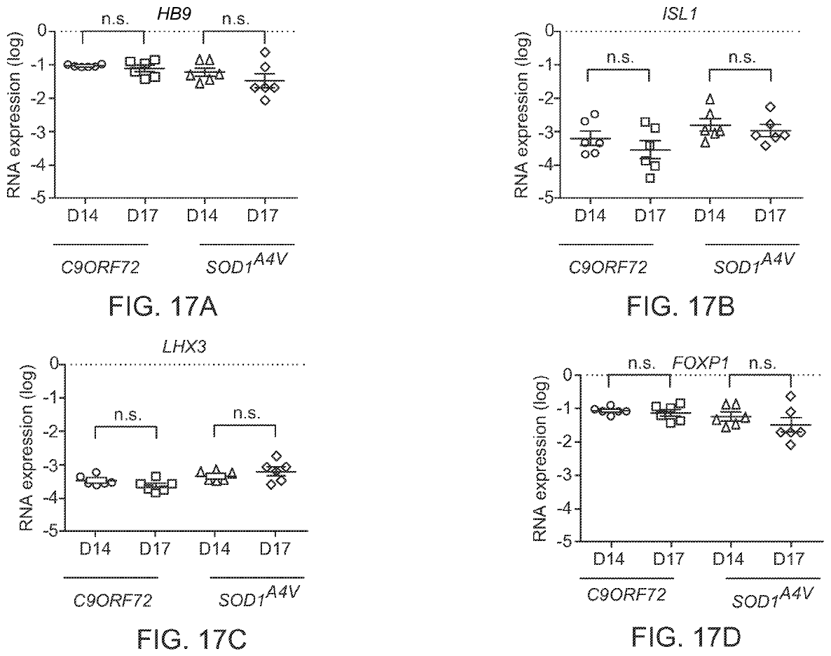

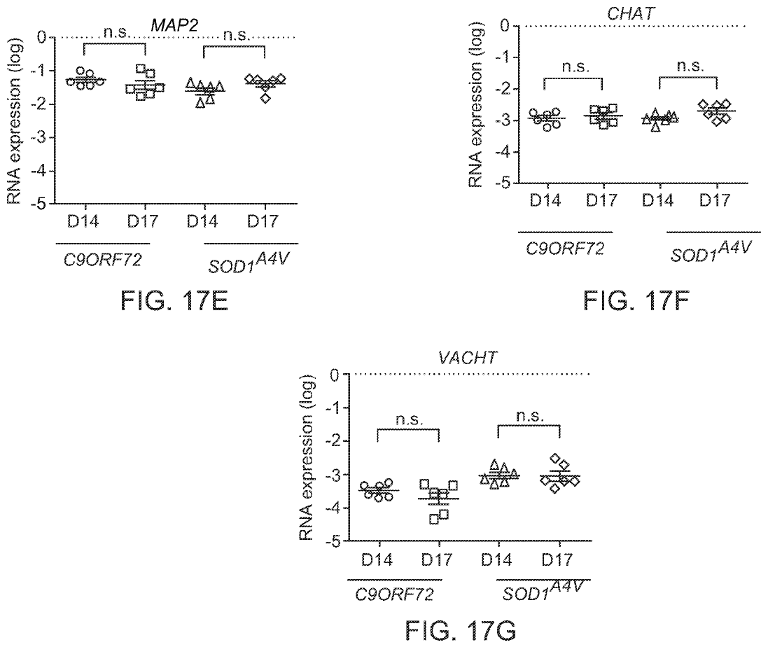

To compare overall transcriptomic patterns between oMN-like and sMN subtypes, a sMN-specific genetic reporter system was developed from ALS hiPSCs. HB9 is known to be a specific transcriptional marker for sMN specification in the spinal cord^{61,62}. Indeed, whole mount staining of Hb9::GFP⁶³ and Isl1::GFP⁶⁴ transgenic embryos clearly showed that projection of Isl1::GFP⁺ cell populations was identical to Hb9::GFP⁺ cells in the spinal cord, but not oculomotor neuronal projection in the midbrain^{65,66} (FIGS. 15A-B′). These data revealed the specificity of Hb9 expression for developing sMN populations in mouse embryo. Thus, an HB9::GFP genetic reporter was generated in ALS hiPSC lines (SOD1^{A4V} and C9ORF72) using the CRISPR-Cas9 system (FIGS. 15C-D′) and the stop codon in the human HB9 locus was replaced with 2A-eGFP-PGK-Puro gene cassette (FIG. 15E). After morphological and antibiotic selection, sMN differentiation using an established sMN differentiation method as previously described⁶⁷ was attempted (FIG. 15F). FACS analysis was used to confirm high numbers of HB9::GFP⁺ cells in differentiated culture of both HB9)::GFP⁺ genetic reporter ALS hiPSC lines (FIGS. 1H-I). In addition, time course analysis of HB9::GFP⁺ cells by FACS indicated that the GFP expression gradually increased beginning at Day 5, but then started to decrease after Day 13 till Day 17 in both ALS lines. qRT-PCR analysis using post-sorted HB9::GFP⁺ cells (at Day 17) showed highly enriched mRNA expression of sMN-specific genes, including HB9, ISL1, LHX3, FOXP1, TBX20, CHAT and VACHT, and significant down-regulation of pluripotent markers, OCT4 and NANOG (FIGS. 15G-I and FIG. 5), which demonstrated that the HB9::GFP+ cells are indeed enriched with sMN-specific molecular markers. To identify subtype-specific susceptibility of ALS sMN differentiation, comparable levels of subtype specific marker gene expression (HB9, ISL1, MAP2, CHAT and VACHT for sMN, LHX3 for medial motor column, FOXP1 for lateral motor column) were found at D17 and earlier time (D14), suggesting that the proportion of sMN subtypes may maintain (FIG. 17). FACS analysis also indicated (FIGS. 1H-I and FIGS. 16H-I) that the majority of cells co-expressed HB9::GFP and HB9 (96.7% in C9ORF72, 85.3% in SOD1^{A4V}) as well as ISL1 and HB9::GFP (96.6% in (9ORF72, 88.8% in SOD1^{A4V}).

Additionally, it was determined that there are increased expression levels of different HOX subfamily genes (HOXA2, 5, 7 and 10, detected by qRT-PCR) in the HB9::GFP⁺ cells, but not in PHOX2B::GFP⁺ cells, supporting the regional specificity of both neuronal populations (FIG. 14I). Taken together, these data indicate that HB9::GFP⁺ cells derived from two different ALS hiPSC lines serve as sMN populations.

FIGS. 15A-I are images, schematics, karyotypes, data graph and heatmaps demonstrating how HB9::GFP reporter in SOD1^{A4V} and C9ORF72 ALS lines was generated according to one embodiment. FIGS. 15A-B′ disclose wholemount GFP expression of Hb9 and Isl1 transgenic mouse at E11.5 embryo with magnified view as indicated in (A′) and (B′). FIGS. 15C-D show representative images and karyotypes of SOD1^{A4V} and C9ORF72 HB9B::GFP reporter lines. FIG. 15E is a description of HB9 gene targeting using CRISPR-Cas9 homologous recombination. FIG. 15F provides a schematic protocol of sMN cell differentiation. FIG. 15G shows the time course GFP expression of sMN differentiation in SOD1^{A4V}|C90RF72 and HB9 antibody-stained cells of control hiPSC line by FACS analysis (at least n=3 for each group: technical replicates: n.s.: not significant, *P<0.05, **P<0.01, ***P<0.001: unpaired student's t-test). (FIG. 15H) Heatmap presents characteristic marker expression of ES (OCT4 and NANOG) and sMN (HB9, ISL1, LHX3, CHAT and FOXP1) in post-sorted OCT4::GFP⁺, SOD1^{A4V} HB9::GFP⁺ and C9ORF72 HB9::GFP⁺ cells by qRT-PCR (n=3 for each group: technical replicates). (FIG. 15I) A heatmap presents the gene expression levels of different spinal axis region markers (HOXA2, A5, A7 and A10) and cell type specific makers (HB9 for sMN and PHOX2A and TBX20 for oMN-like) in post-sorted PHOX2B::GFP⁺ and HB9::GFP⁺ cells of SOD1^{A4V} and C9ORF72 ALS hiPSCs by qRT-PCR (n=3 for each group: technical replicates). Scale bars: 2000 μm (A-B′) or 100 μm (C-D), Error bars: mean±SEM.

FIGS. 16A-16I are data graphs and FACS dot displays showing the expression of sMN specific markers in SOD1^{A4V} and C9ORF72 derived HB9::GFP⁺ cells according to an embodiment. (FIGS. 16A-G) qRT-PCR analysis indicates sMN characteristic transcripts (HB9, ISL1, LHX3, CHAT and FOXP1), but not hiPSC characteristic transcripts (OCT4 and NANOG) in HB9::GFP⁺ cells of SOD1^{A4V} and C9ORF72 (D14) (at least n=5 for each group: technical replicates: *P<0.05, **P<0.01, ***P<0.001, ****P<0.0001, n.s.: not significant: unpaired student's t-test). (H-I) HB9 (C9ORF72: 96.7%, SOD1^{A4V}: 85.3%) and ISL1 (C9ORF72: 96.6%, SOD1^{A4V}: 88.8%) stained cells are highly co-expressed with HB9::GFP⁺ of both ALS derived sMN by FACS analysis (D14). Error bars: mean±SEM.

FIGS. 17A-17G are graphs showing the characterization of sMN subtypes by maker expression in different differentiation time of C9ROF72 and SOD1^{A4V} ALS hiPSC lines according to an embodiment. (FIGS. 17A-G) qRT-PCR results present comparable expression of sMN specific (HB9 and ISL1), subtype specific (FOXP1 for later motor column and LHX3 for medial motor column) maker expression and maturation (MAP2, CHAT and VACHT) in differentiation day 14 and 17 of C9ROF72 and SOD1^{A4V} lines (n.s.: not significant: unpaired student's t-test). Error bars: mean±SEM.

Transcriptomic differences in lipid metabolism between PHOX2B::GFP⁺ oMN-like and HB9::GFP⁺ sMN populations in C9ORF72 and SOD1^{A4V} ALS lines were revealed by comparative transcriptome profiling

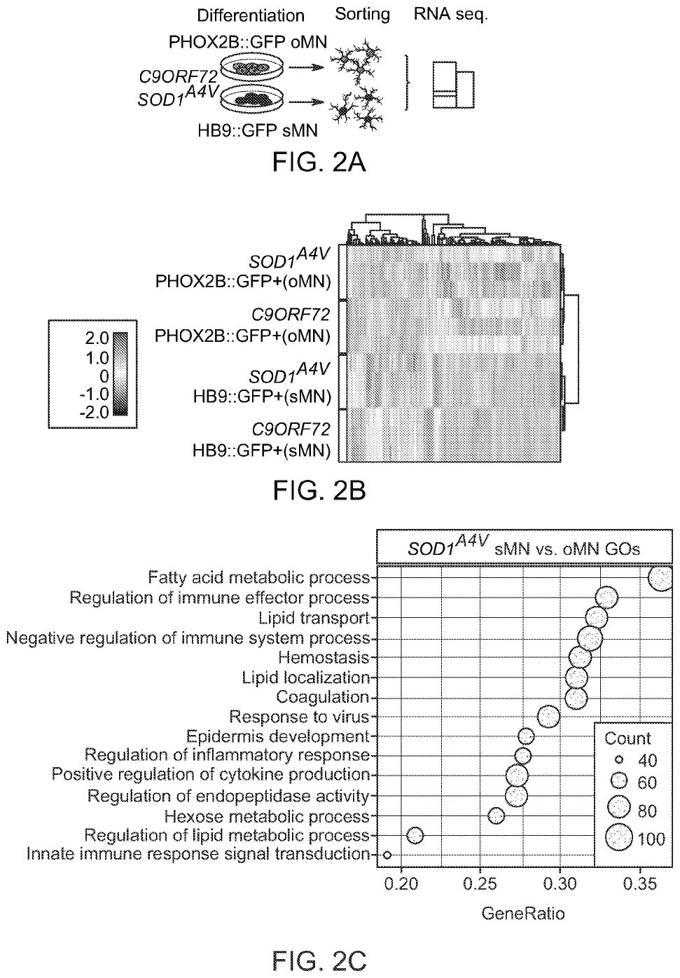

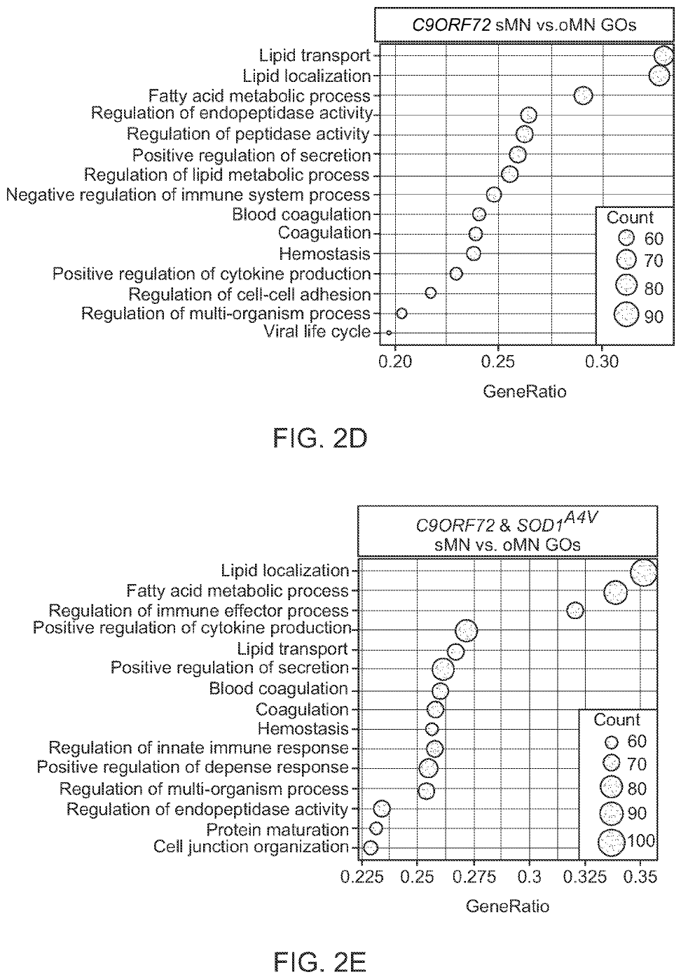

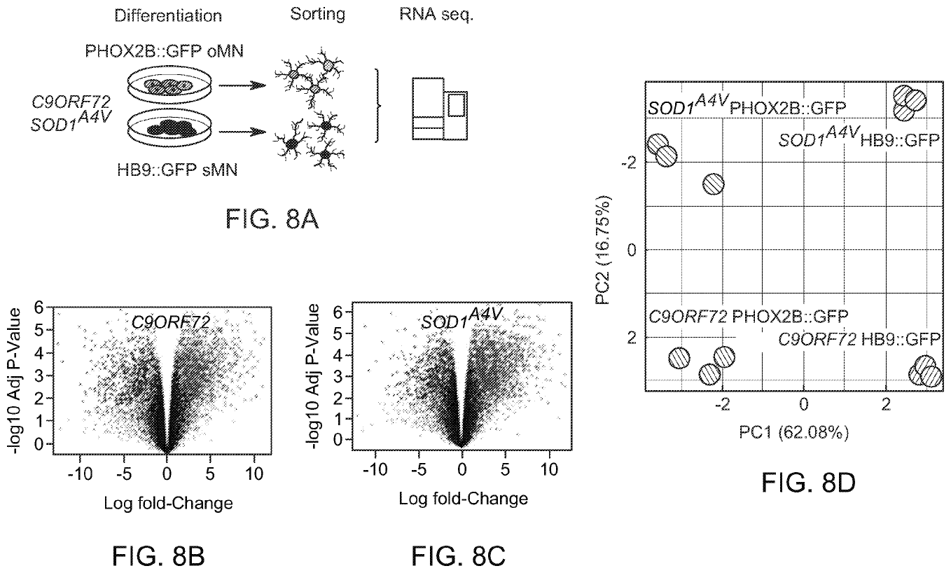

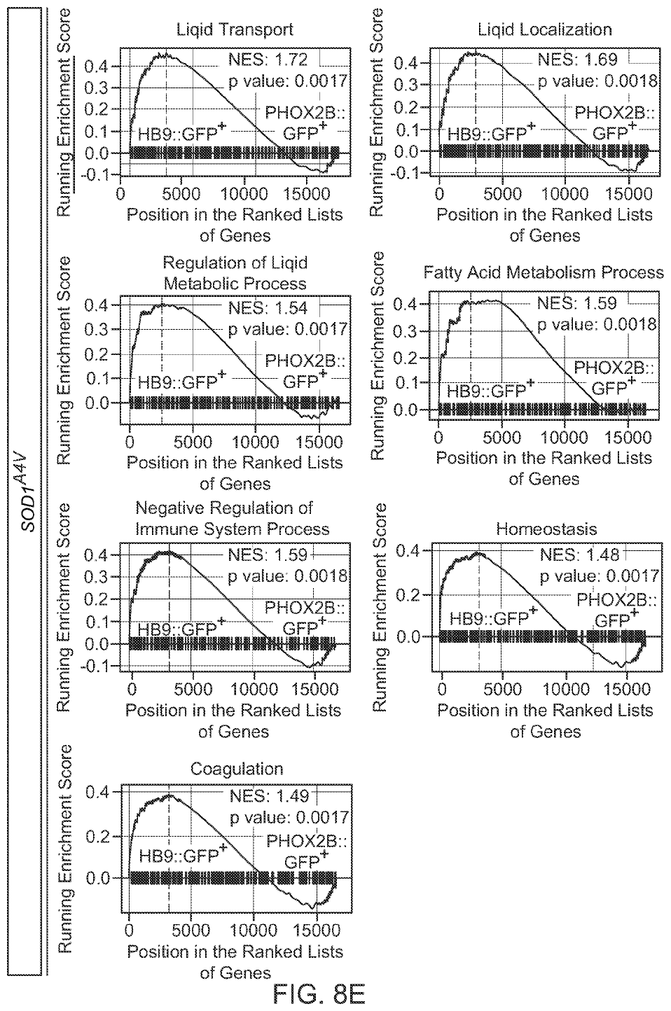

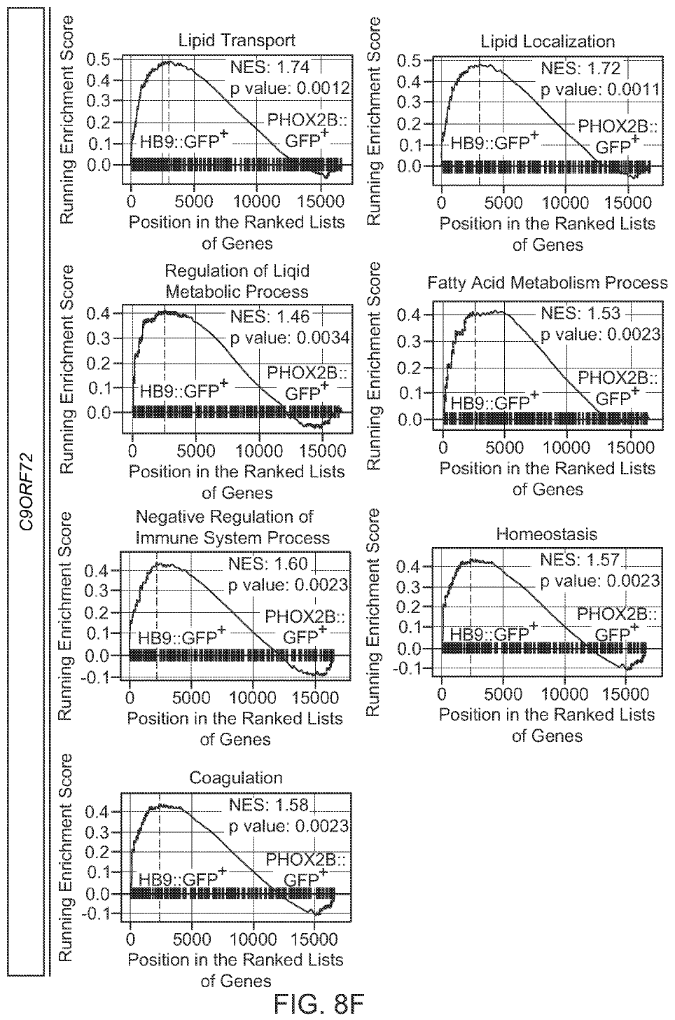

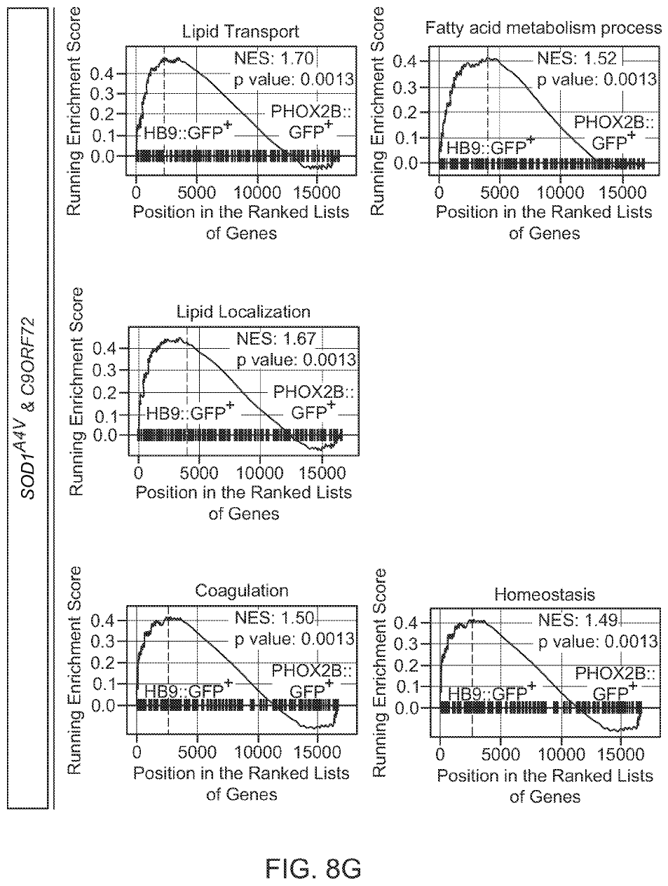

In order to identify intrinsic properties that explain selective vulnerability in sMN subtypes, unbiased transcriptome analysis was performed using post-sorted PHOX2B: GFP⁺ oMN-like and HB9::GFP⁺ sMN cells derived from C9ORF72 and SOD1^{A4V} ALS lines (FIG. 2A and FIG. 8A). In time course analysis of HB9::GFP expression, the GFP signal gradually decreased after Day 13 (SOD1^{A4V}) and Day 17 (C9ORF72) of the sMN specification (FIG. 15G). Thus, 3 different batches of differentiated PHOX2B::GFP⁺ and HB9::GFP⁺ cells were harvested at Day 17 for FACS purification.

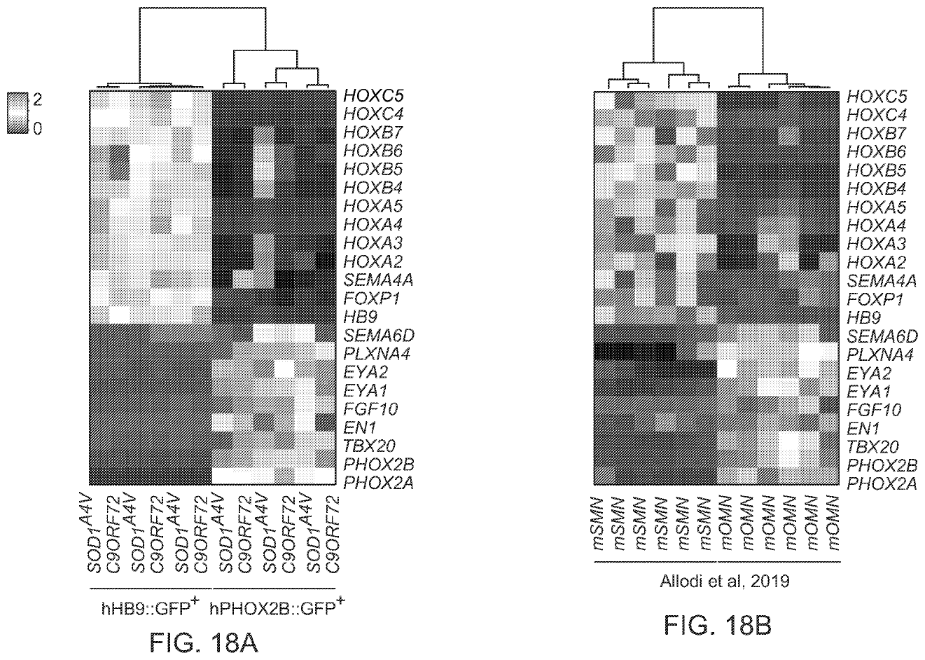

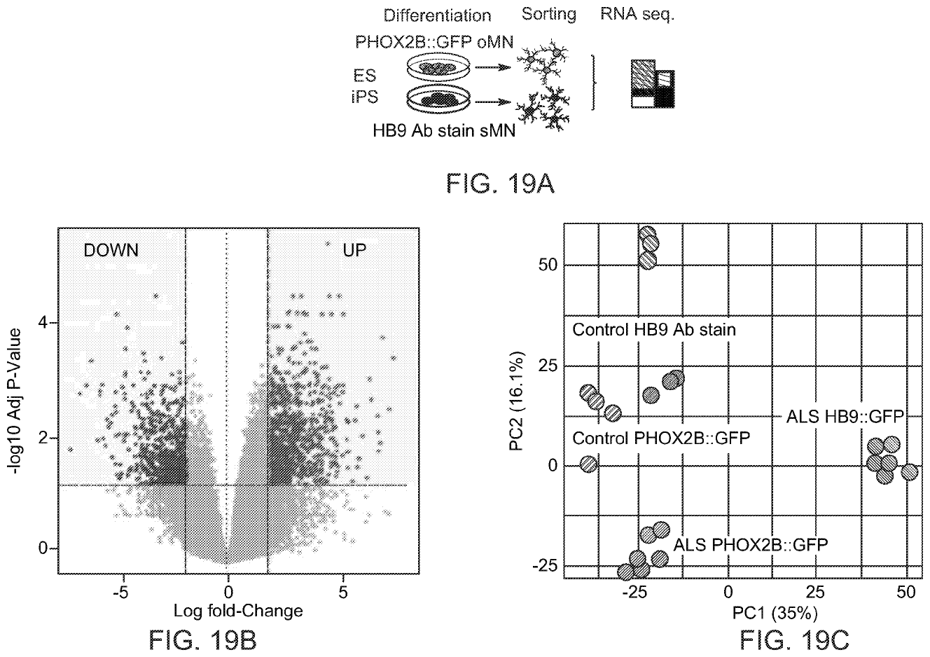

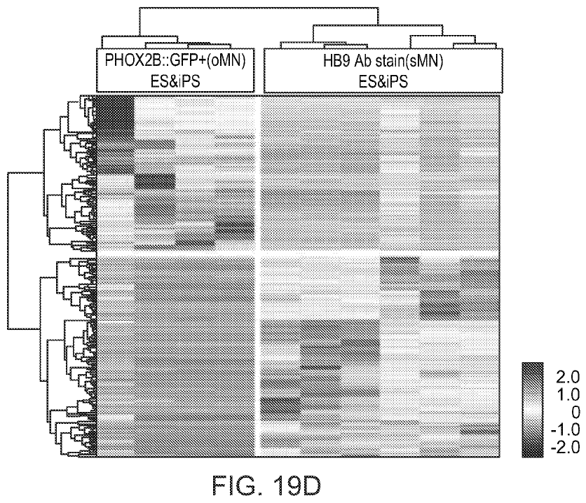

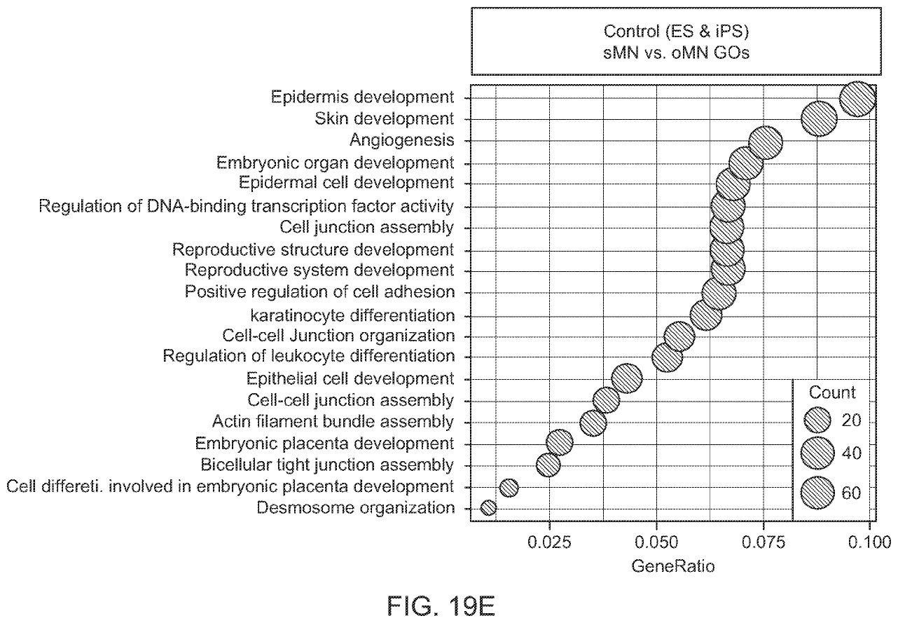

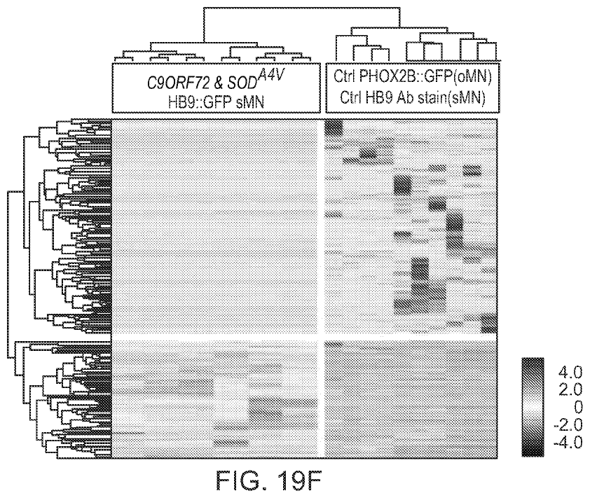

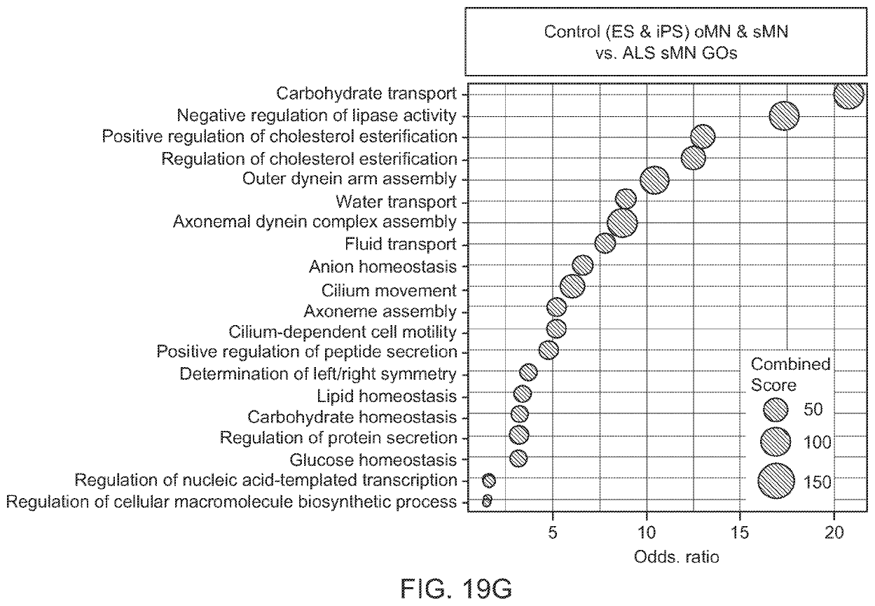

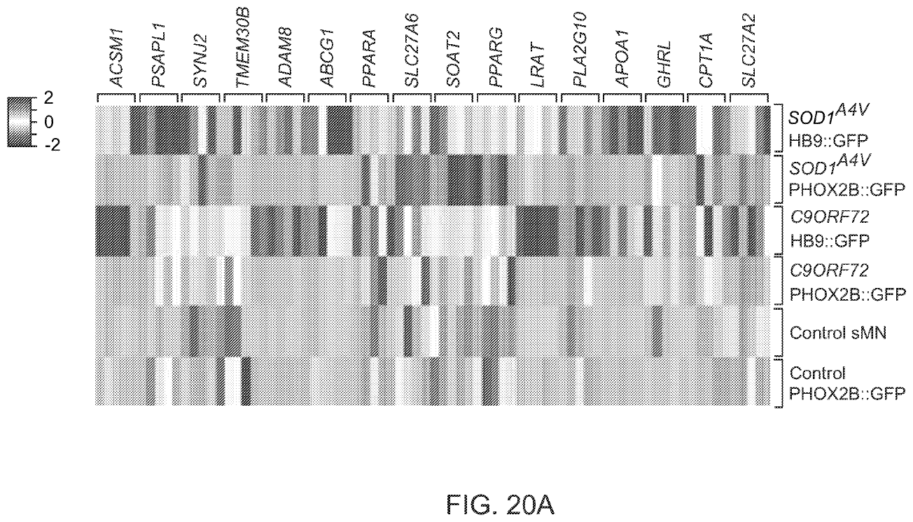

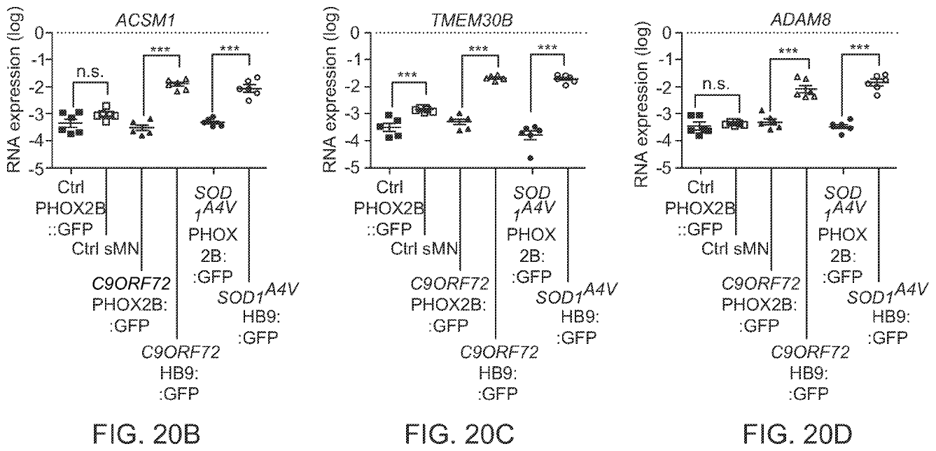

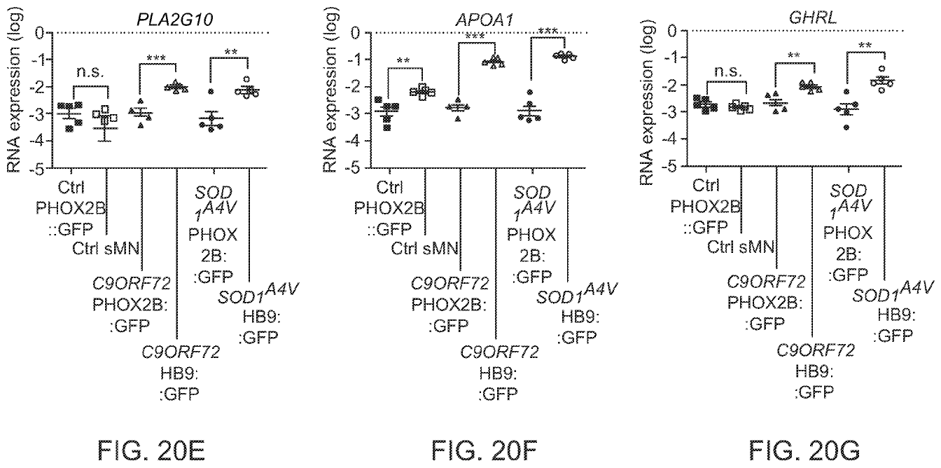

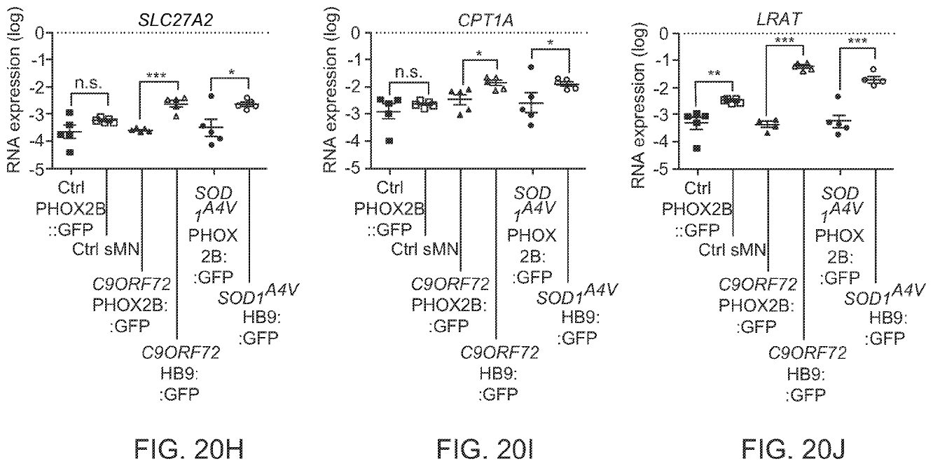

Firstly, for further confirmation of the cellular identities of PHOX2B::GFP⁺ oMN-like and HB9::GFP⁺ sMNs, the RNA-sequencing data were compared with a published data set⁶⁸ where transcriptomic differences between oMN and sMN were shown based on the other published data^{35,69}, including oMN markers (PHOX2A, PHOX2B, TBX20, EN1, FGF10, EYA1, EYA2, PLXNA4 and SEMA6D) and sMN markers (HB9, FOXP1, SEMA4A, HOXA2, HOXA3, HOXA4, HOXA5, HOXB4, HOXB5, HOXB6, HOXB7, HOXC4 and HOXC5) (FIGS. 18A-B). Next, to identify common target pathways of selective vulnerability between two types of motor neurons of SOD1^{A4V} and C9ORF72 lines, focus shifted to differentially enriched gene cohorts of the HB9::GFP⁺ or PHOX2B::GFP⁺ populations of SOD1^{A4V} and C9ORF72 background (total 4 groups: SOD1^{A4V} HB9::GFP⁺, SOD1^{A4V} PHOX2B::GFP⁺, C9ORF72 HB9::GFP⁺, and C9ORF72 PHOX2B::GFP⁺ cells with 3 technical replicates) (FIGS. 8B-8D). PHOX2B::GFP⁺ and HB9)::GFP⁺ cells derived from SOD1^{A4V} and C9ORF72 lines showed clearly distinct expression patterns of enriched genes (FIG. 2B). To identify meaningful pathways that are associated with cell-type specific vulnerability, gene set enrichment analysis (GSEA) using ranked list of genes by the degree of their expression was used. HB9::GFP⁺ enriched Gene Ontology (GO) terms were selected over GO terms of PHOX2B::GFP⁺ populations, which were statistically significant in ALS lines (FIG. 2C-D). By screening for convergent target pathways in two different ALS lines of HB9::GFP⁺. common GO terms between gene set hierarchies of each ALS mutation were identified. Interestingly, genes involved in lipid metabolic pathways such as ‘Lipid Transport (GO:0006869)’, ‘Lipid Localization (GO:0010876)’, ‘Regulation of Lipid Metabolic Process (GO:0019216)’ and ‘Fatty Acid Metabolic Process (GO:0006631)’ were commonly enriched in both ALS lines, but the other common GOs did not show any pathway correlation (FIGS. 8E-8F). To rigorously confirm whether the lipid related GO terms mentioned above can represent each cell type of ALS lines, transcripts of PHOX2B::GFP⁺ oMN-like and HB9::GFP⁺ sMNs were compared, regardless of the SOD1^{A4V} and C9ORF72 mutations and found that the significantly enriched GO terms were relevant to lipid metabolism pathways in both ALS-derived HB9::GFP⁺ cells (FIG. 2E and FIG. 8G). However, such aberrant lipid metabolism was not found in the transcriptional comparison between oMN and sMN population derived from healthy control hESC/hiPSC lines (FIG. 19). RNA-sequencing data was also confirmed by qRT-PCR (with an additional 3 technical replicates) with specific primer sets for lipid metabolism related genes (ACSM1, TMEM30B, ADAM8, PLA2G10, APOA1, GHRL, SLC27A2, CPTIA and LRAT) by showing statistically enriched expression of lipid metabolism related transcripts in HB9::GFP⁺ ALS lines (FIGS. 20A-J). Importantly, the expression patterns of identified genes were similar between the oMN-like and sMNs culture of healthy hESCs, indicating that aberrant transcriptional changes in lipid metabolism are specific to ALS pathogenesis. Taken together, these data strongly indicate that significantly altered lipid metabolism related pathways in HB9::GFP⁺ cells of SOD1^{A4V} and C9ORF72 ALS hiPSCs are a prospective target to elucidate sMN pathology in ALS.

FIGS. 2A-2E show an illustration and data graphs showing that genome-wide RNA sequencing analysis reveals aberrant lipid metabolism after comparison between post-sorted HB9::GFP⁺ and PHOX2B::GFP⁺ in SOD1^{A4V} and C9ORF72 ALS lines according to an embodiment. (FIG. 2A) Illustration of transcriptome profiling of HB9::GFP⁺ versus PHOX2B::GFP⁺. (FIG. 2B) Heatmap indicates differentially expressed genes between PHOX2B::GFP⁺ and HB9::GFP⁺ in SOD1^{A4V} (n=3 for each group: technical replicates) and C9ORF72 ALS lines (n=3 for each group: technical replicates). (FIGS. 2C-2E) Dot plots represent Top 15 gene sets over-represented in HB9::GFP⁺ compared to PHOX2B::GFP⁺. Single ALS lines were analyzed in panel C and D for SOD1^{A4V} and C9ORF72, respectively. Those two lines were combined and analyzed together in panel E to validate the data. Individual dots are sized to reflect the number of genes in each gene set.

FIGS. 8A-8G show an illustration and data graphs showing that transcriptome profiling reveals differences between PHOX2B::GFP⁺ and HB9::GFP⁺ cells in both SOD1^{A4V} and C90RF72 ALS lines according to an embodiment. (FIG. 8A) Illustration of transcriptome profiling of HB9::GFP⁺ versus PHOX2B::GFP⁺. (FIGS. 8B-8C) Volcano plots indicate a substantial transcriptomic difference between HB9::GFP and PHOX2B::GFP in both SOD1^{A4V} and C9ORF72 ALS lines (n=3 for each group: technical replicates) (see Methods section for details). (FIG. 8D) Principal component analysis (PCA) plot represents distinct clustering between HB9::GFP and PHOX2B::GFP cell types derived from both SOD1^{A4V} and C9ORF72 ALS lines (n=3 for each group: technical replicates). (FIGS. 8E-8F) Gene set enrichment analysis (GSEA) plots show commonly over-represented GO terms of HB9::GFP⁺ cells compared to PHOX2B::GFP⁺ cells in both SOD1^{A4V} and C9ORF72 ALS lines (n=3 for each group: technical replicates). (FIG. 8G) Combined dataset of the two ALS lines consistently show the same over-represented GO terms as observed in single ALS line datasets (n=3 for each group: technical replicates).

FIGS. 18A-B are heatmaps showing differential expression levels of oMN- or sMN-specific genes in sorted HB9::GFP and PHOX2B::GFP⁺ of SOD1^{A4V} and C9ORF72 ALS hiPSC lines (FIG. 18A), or reanalyzed mouse dataset from a previous literature (FIG. 18B).

FIG. 19A-G are a schematic, heat maps and graphs showing metabolomics analysis comparing the transcriptome profiles of healthy hESC and hiPSC-derived PHOX2B::GFP⁺ cells and HB9 antibody-stained cells according to an embodiment. FIG. 19A is an Illustration of transcriptome profiling of HB9::GFP⁺ versus PHOX2B::GFP⁺ in hESC and hiPSC lines. (FIG. 19B) Volcano plots indicate a substantial transcriptomic difference between HB9 stained cells and PHOX2B::GFP⁺ cells in control (hESC+hiPSC) lines (at least n=4 for each group: technical replicates) (see Methods section for details). (FIG. 19C) Principal component analysis (PCA) plot represents distinct clustering between HB9::GFP/antibody-stained and PHOX2B::GFP cell types derived from ALS (C9ORF72+SOD1^{A4V}) and control (hESC+hiPSC) lines (at least n=4 for each group: technical replicates). (FIG. 19D) Heatmap indicates differentially expressed genes between PHOX2B::GFP⁺ and HB9 antibody-stained cells in control (hESC+hiPSC) lines (at least n=4 for each group: technical replicates). (FIG. 19E) Dot plot shows commonly over-represented GO terms of HB9::GFP⁺ cells compared to PHOX2B::GFP⁺ cells in control (hESC+hiPSC) lines (at least n=4 for each group: technical replicates). (FIG. 19F) Heatmap indicates differentially expressed genes between combined PHOX2B::GFP⁺ and HB9 antibody-stained cells in control (hESC+hiPSC) lines (at least n=4 for each group: technical replicates) and HB9::GFP⁺ in ALS hiPSCs (C9ORF72+SOD1^{A4V}) (n=3 for each group: technical replicates). (FIG. 19G) Combined dataset of the two ALS lines show the same over-represented GO terms comparing to combined PHOX2B::GFP⁺ and HB9 stained cells of control lines (hESC+hiPSC) (at least n=4 for each group: technical replicates).

FIGS. 20A-D are heat maps and data graphs showing abnormal expression of lipid related transcripts in SOD1^{A4V} and C9ORF72 ALS lines by qRT-PCR analysis according to an embodiment. (FIG. 20A) Heatmap shows enriched transcripts in sorted HB9::GFP⁺ of SOD1^{A4V} and C9ORF72, but not sorted control and PHOX2B::GFP⁺. (FIGS. 20B-J) Each individual plot indicates altered expression transcripts in post-sorted HB9::GFP⁺ of SOD1^{A4V} and C9ORF72 (*P<0.05, **P<0.01, ***P<0.001, n.s.: not significant, unpaired student's t-test, at least n=3 for each group: technical replicates). Error bars: mean±SEM.

Unbiased metabolomics analysis corroborated transcriptional discrepancies in ALS-specific PHOX2B::GFP⁺ oMN-like and HB9::GFP⁺ sMNs cells

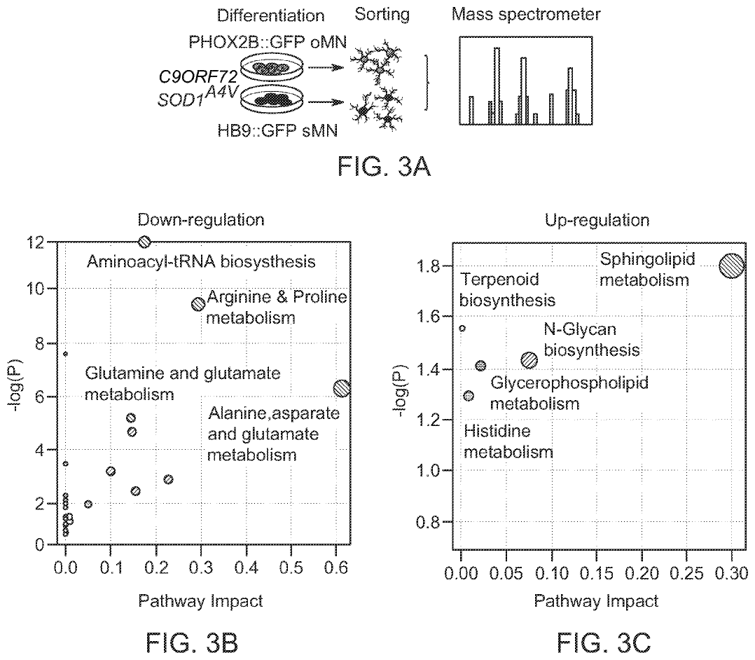

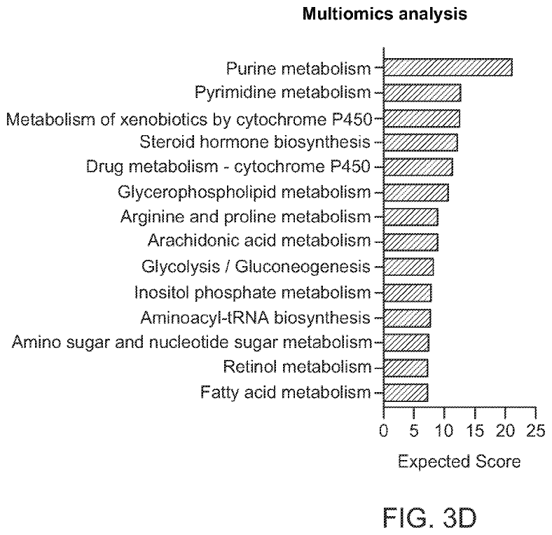

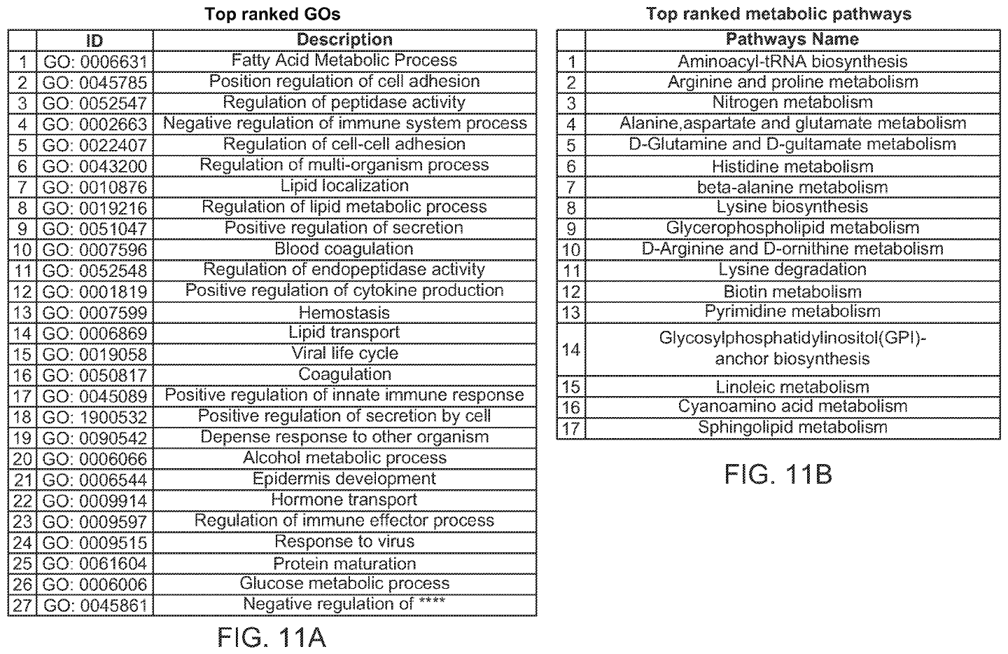

To verify the RNA sequencing data and pinpoint target pathways that are involved in cell type specific phenotypic vulnerability, studies were taken to identify metabolic differences by performing liquid chromatography mass spectrometry (LC/MS) using post-sorted PHOX2B::GFP⁺ cells and HB9::GFP⁺ cells of both SOD1^{A4V} and C9ORF72 ALS hiPSC lines (total 4 groups: SOD1^{A4V} HB9::GFP⁺, SOD1^{A4V} PHOX2B::GFP⁺, C9ORF72 HB9::GFP⁺, and C9ORF72 PHOX2B::GFP⁺ cells with 3 technical replicates) (FIG. 3A). Metabolic discrepancies between the PHOX2B::GFP⁺ cells and HB9::GFP⁺ cells were determined by comparing the relative abundance of ˜3,000 metabolites selected from the Metlin database (http:://metlin.scripps.edu). Using MetaboAnalyst v.4.0, multivariate unbiased clustering analyses identified a subset of metabolites and mapped annotated pathways. The pathway mapping analysis revealed that transporters and metabolic pathways for most amino acids such as arginine, proline, glutamine, glutamate, alanine, and aspartate belonged to relatively down-regulated pathways in HB9::GFP⁺ cells compared to those in PHOX2B::GPF⁺ cells (FIG. 3B). Amino acid deficits with activated aerobic glycolysis were previously reported to be associated with defective energy metabolism in a murine cellular model of ALS⁷⁰, implying the reproducibility of the models. Intriguingly, the analysis showed that various lipid metabolic pathways such as sphingolipid metabolism, glycerophospholipid metabolism and terpenoid biosynthesis as groups that belong to the most aberrantly up-regulated pathways in HB9::GFP⁺ cells as compared to those in PHOX2B::GFP⁺ cells (FIG. 3C). These findings are corroborated by the RNA-seq data. A multi-omics strategy using foregoing metabolomics and transcriptomics was used to combine two different “omics to statistically identify functional association between them and pinpointed pathways that are perturbed among the lipid metabolic pathways of HB9::GFP⁺ cells (FIG. 3D and FIGS. 11A and 11B). Intriguingly, the Expected Score by Metabo Analyst v.4.0⁷¹ also supported the findings by presenting lipid metabolism related pathways including ‘steroid hormone biosynthesis’, ‘glycerophospholipid metabolism’, ‘arachidonic acid metabolism’ and ‘fatty acid metabolism’ as top-ranked pathways (FIG. 3D). Thus, extensive multi-omics analysis using metabolomics and transcriptomics results indicated that lipid-related metabolism pathways were significantly perturbed in both SOD1^{A4V} and C9ORF72 HB9::GFP⁺ cells of ALS lines and might serve as potential targets to identify new pharmacological treatments.

FIGS. 3A-3D show an illustration and data graphs showing metabolomics analysis that indicates up-regulation of lipid metabolism in post sorted HB9::GFP⁺ of SOD1^{A4V} and C9ORF72 ALS lines according to an embodiment. (FIG. 3A) Schematic illustration of post-sorted metabolomics analysis. (FIGS. 3B-3C) Pathway analysis by MetaboAnalyst v.4.0 shows up-regulated and down-regulated metabolisms (n=3 for each group). (FIG. 3D) Glycerophospholipid, fatty acid and arachidonic acid metabolism are highly enriched in expected score of multi-omics analysis (n=3 for each group: technical replicates).

FIGS. 11A and 11B are lists of the top ranked perturbed pathways according to an embodiment.

Activation of arachidonic acid pathway is a common metabolic signature of sMN cells with various genetic backgrounds

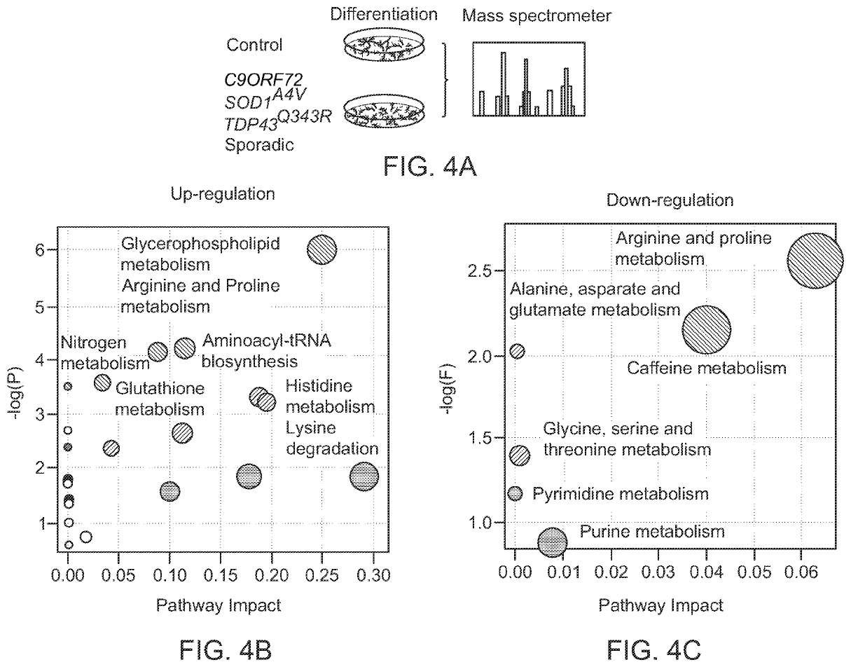

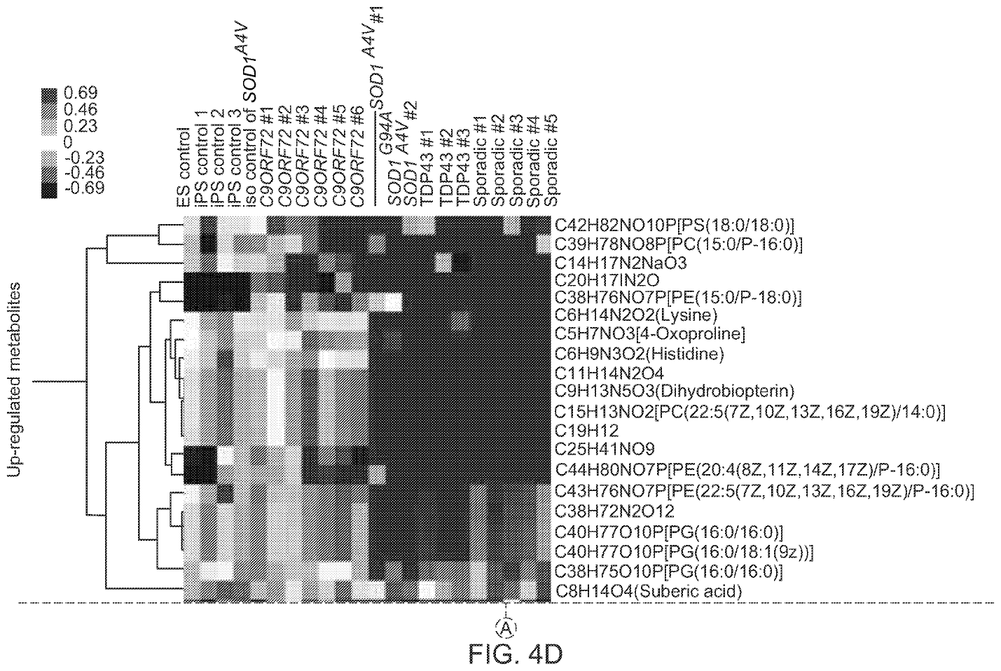

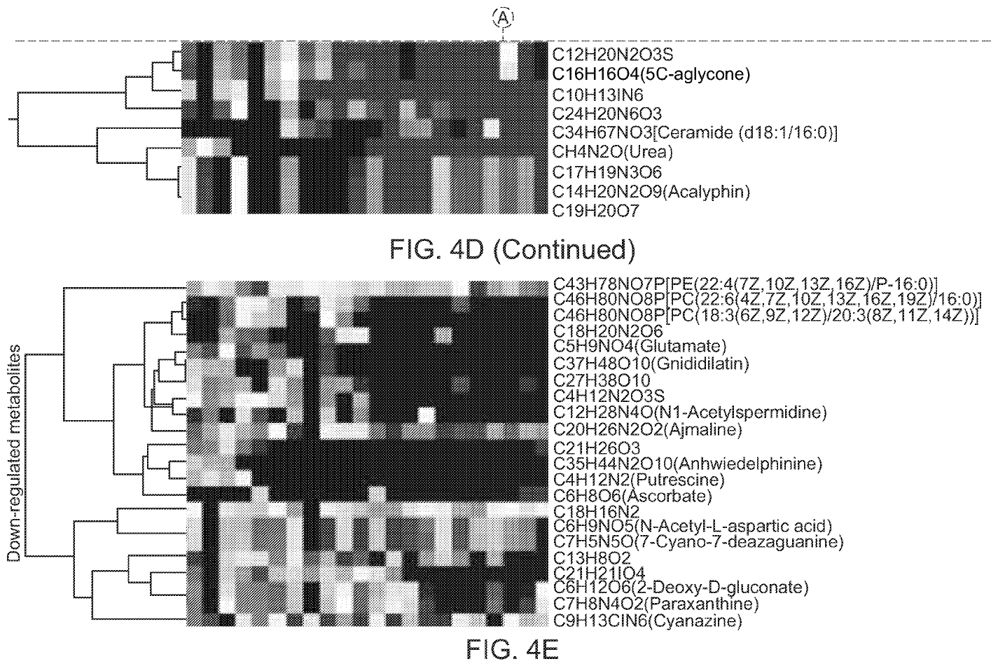

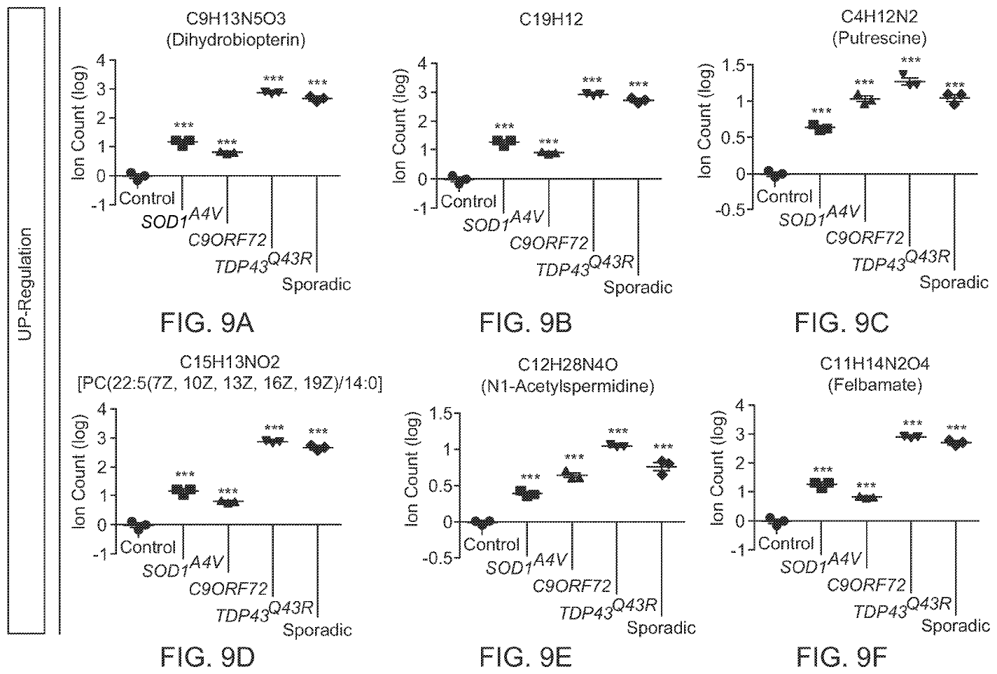

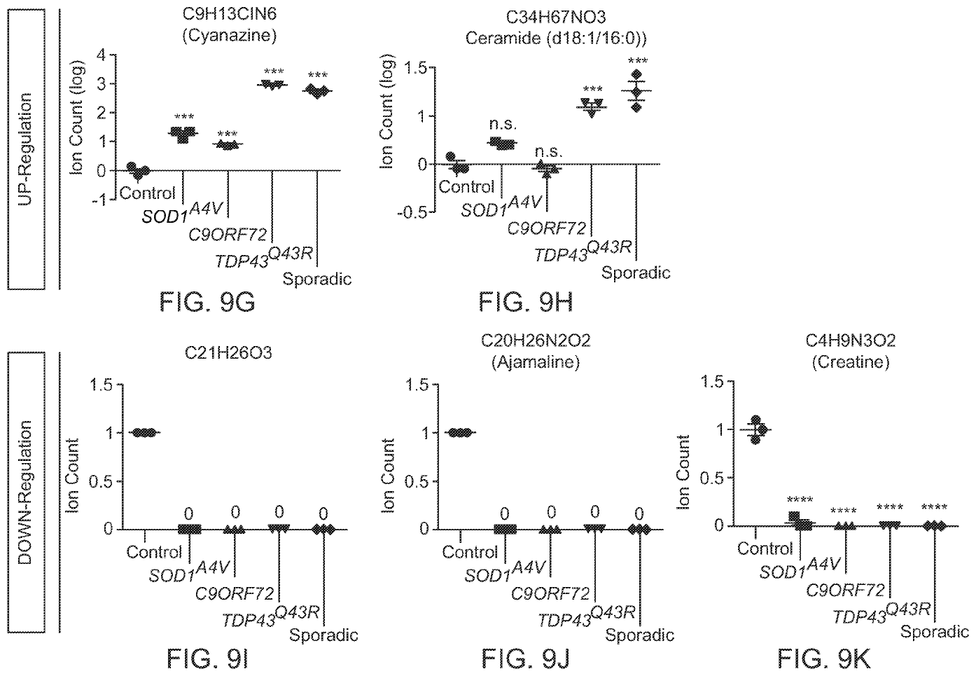

After the unbiased multi-omics analysis using two ALS lines, it was asked whether this phenotype is common in different mutations and sporadic ALS. Thus, a more stringent experimental plan was set to analyze the ‘unsorted’ crude sMN culture of multiple ALS hiPSC lines (FIG. 4A). A focused metabolomics analysis was performed using ˜600 selected lipid metabolite references with unsorted samples of SOD1^{A4V}, C9ORF72, TDP43^{Q343R} and sporadic ALS lines compared to healthy control group, (FIGS. 4B-4E, FIGS. 9A-9K) (each group had 3 independent technical replicates). This focused analysis identified highly enriched metabolites belonging to glycerophospholipid metabolism, which is corroborated by results of the RNA sequencing (FIG. 2C-E) and post-sorted untargeted metabolic analysis (FIG. 3C). Therefore, the dysregulated lipid related metabolism of sMNs derived from four ALS hiPSC lines with various genetic backgrounds seem to be potentially common pathways in ALS pathogenesis.

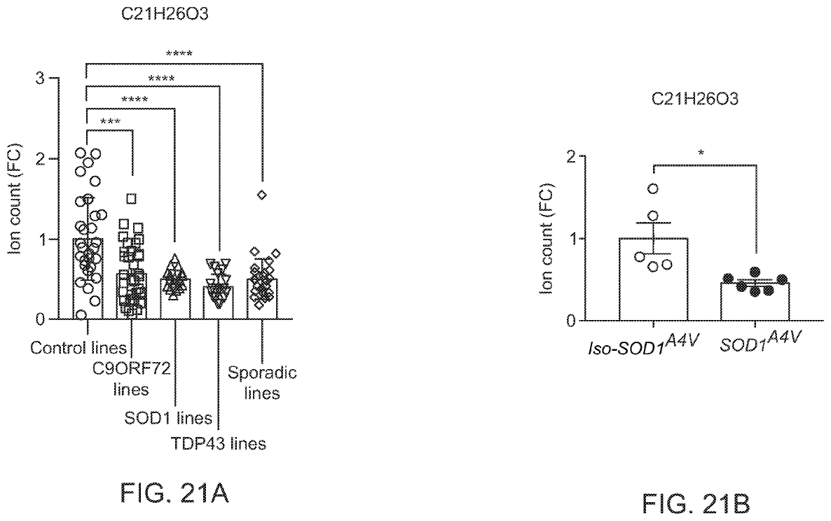

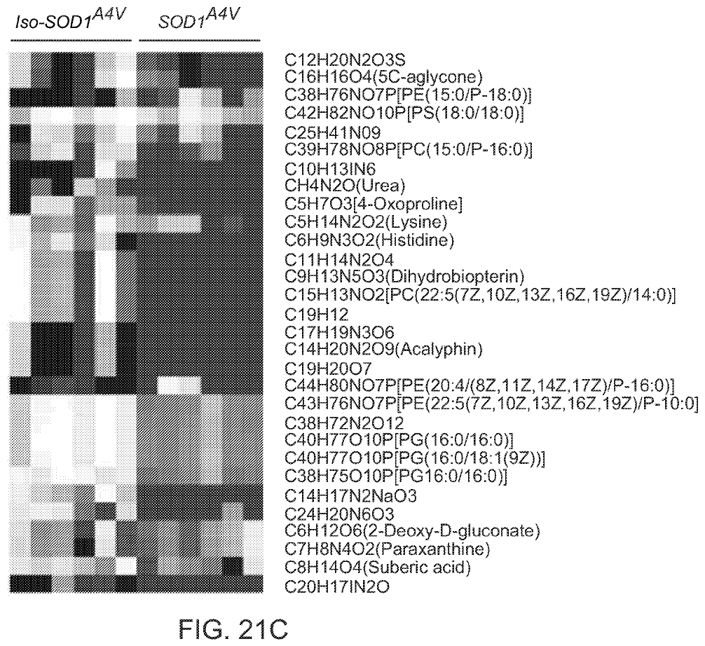

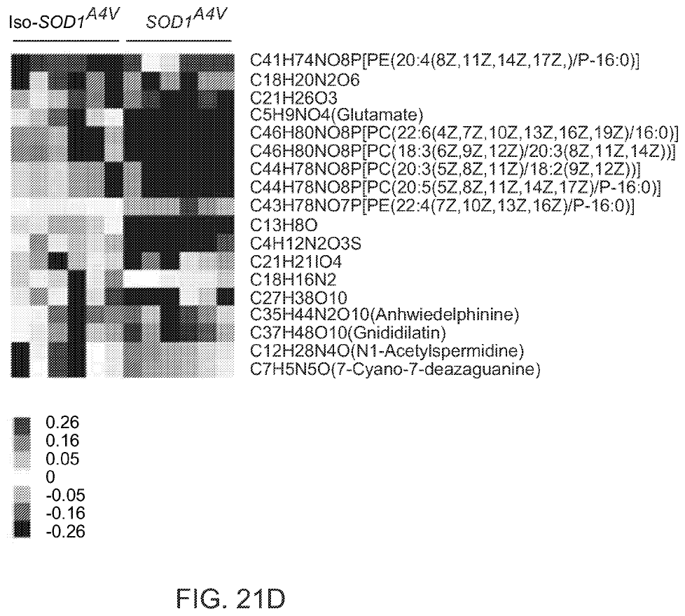

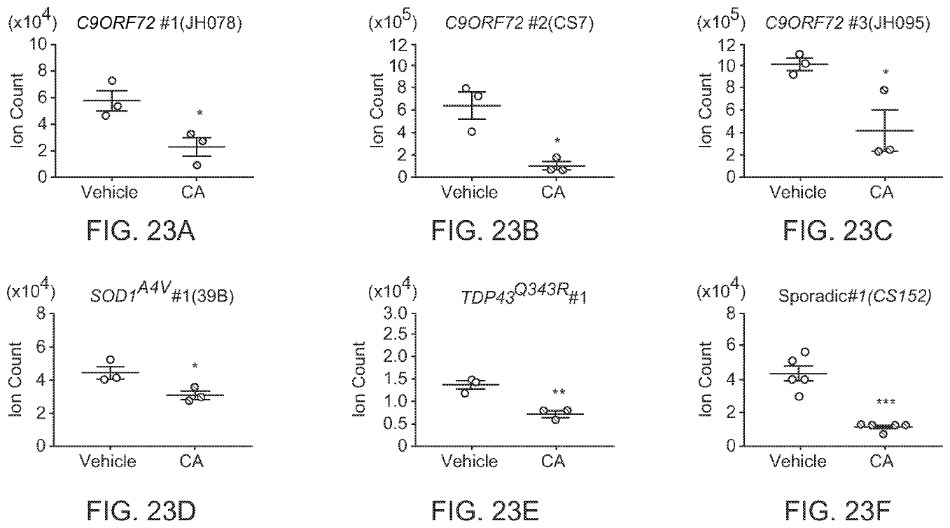

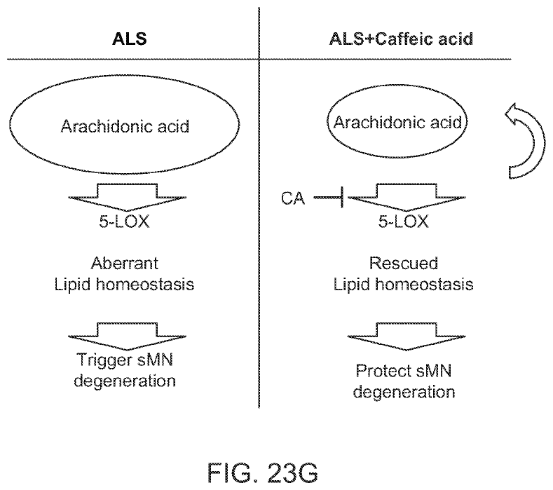

To pinpoint specific pathways that are associated with ALS sMN pathology, commonly over-represented or nearly absent metabolites were first classified by statistical analysis and it was determined that 29 metabolites belonged to the highly represented group and 22 metabolites belonged to the downregulated group that were common in multiple ALS (C9ORF72, 6 lines: SOD1, 3 lines: TDP 43, 3 lines: sporadic, 5 lines; each line had 3 independent technical replicates) (FIG. 4C-E and FIGS. 9 and 21). Ion count value of each metabolite in sMN culture of SOD1^{A4V} ALS hiPSC line was reversed in isogenic SOD1^{A4V} samples (FIGS. 21C-D). FIGFIG Unsaturated glycerophospholipids with various chain lengths were shown to be upregulated in sMNs, while natural compounds involved in the anti-inflammatory response and antimicrobial activities were downregulated (FIGS. 4B-E), implying significant risk of unbalanced redox state in sMN lines. Interestingly, one of significantly downregulated in all sMN cultures natural compound (C21H26O3 molecular formula) was a structural analog of AA861, a known 5-lipoxygenase (5-LOX) inhibitor (FIG. 4E and FIG. 9I and FIGS. 21A-B and 21D). 5-LOX is involved in the AA pathway that catabolizes various glycophospholipid species into downstream lipid metabolites such as AA and leukotrienes (FIG. 23G). Importantly, the levels of AA was dysregulated in plasma samples of ALS patients based on other publication⁷².

Based upon the metabolomics findings, absence of AA861 analog and the previous metabolomics data of ALS patient plasma, it was speculated that the loss of regulation in biosynthesis of AA may be one of the causes of lipid metabolism dysregulation common in ALS sMN, corroborated with the multi-omics analysis by indicating a significantly presented ‘Arachidonic acid metabolism’ with significant Expected Score (˜9.12 by MetaboAnalyst v.4.0)(FIG. 3D). To test the hypothesis, 5-LOX in AA metabolism was selected as a target for further functional analysis, because 5-LOX activity is also known to negatively be related with AA metabolism activity in other diseases^{73,74}. Taken together, the results demonstrate that altered AA metabolism serves as a prominent common metabolic phenotype in sMN culture of SOD1^{A4V}, C9ORF72, TDP 439343R and sporadic ALS lines.

FIGS. 4A-4E are data graphs showing metabolomics analysis in un-sorted sMN differentiation confirmed up-regulation of lipid metabolism, and provides lipid related metabolic candidates in TDP43^{Q343R}, C9ORF72, SOD1^{A4V} and Sporadic ALS lines according to an embodiment. (FIG. 4A) Schematic illustration of un-sorted metabolomics analysis. Each circle represents a different pathway: circle size and color shade are based on the pathway impact and p-value (red being the most significant), respectively. (FIGS. 4B and 4D) Glycerophospholipid metabolism is highly up-regulated (FIG. 4D) in pathway analysis of unsorted SOD1^{A4V}, C9ORF72, TDP 439343R and Sporadic sMN differentiation. (FIGS. 4C and 4E) Heatmap shows up-regulated and down-regulated metabolites candidates from unsorted multiple lines of ALS sMN differentiation (n=3 for each group: technical replicates: n=1 hESC healthy control, n=3 hiPSC healthy control, n=1 isogenic control hiPSC of SOD1^{A4V}, n=6 C9ORF72 ALS hiPSC lines, n=3 SOD1 ALS hiPSC lines, n=3 TDP43 ALS hiPSC lines, n=5 sporadic ALS hiPSC lines; biological replicates).

FIGS. 9A-9K are data graphs showing selection of altered metabolic candidates by metabolomics analysis in ALS lines according to an embodiment. (FIGS. 9A-11K) Ion count values present commonly up-regulated (FIGS. 9A-9H) and down-regulated metabolites (FIGS. 91-9K) in SOD1^{A4V}, C9ORF72, TDP 439343R. Sporadic ALS and control hESC derived sMN differentiation (n=3 for each group: technical replicates, ‘0’: non-detected, ***P<0.001, n.s.: not significant, unpaired student's t-test). Error bars: mean±SEM.

FIGS. 21A-D are data graphs and heatmaps showing common alteration of C21H26O3 in multiple ALS lines and direct comparison of altered metabolic metabolites by metabolomics analysis in isogenic control of SOD1^{A4V} and SOD1^{A4V} ALS hiPSC lines according to one embodiment. Ion count values present commonly down-regulated C21H26O3 metabolic candidate in multiple ALS lines (FIG. 21A) and direct comparison of isogenic control of SOD1^{A4V} and SOD1^{A4V} lines (FIG. 21B) (*P<0.05, ***P<0.001, ****P<0.0001, n.s.: not significant, unpaired student's t-test: at least n=3 for each lines: technical replicates: n=1 hESC control, n=3 hiPSC control, n=1 isogenic control hiPSC of SOD1^{A4V}, n=6 C9ORF72, n=3 SOD1, n=3 TDP43, n=5 sporadic ALS hiPSC: biological replicates). (FIGS. 21C-D) Heatmap shows the lists of selective metabolite candidate in isogenic control of SOD1^{A4V} and SOD1^{A4V} ALS hiPSC lines (n=6 for each group: technical replicates). Error bars: mean±SEM.

Arachidonic acid pathway alterations cause ALS phenotypes

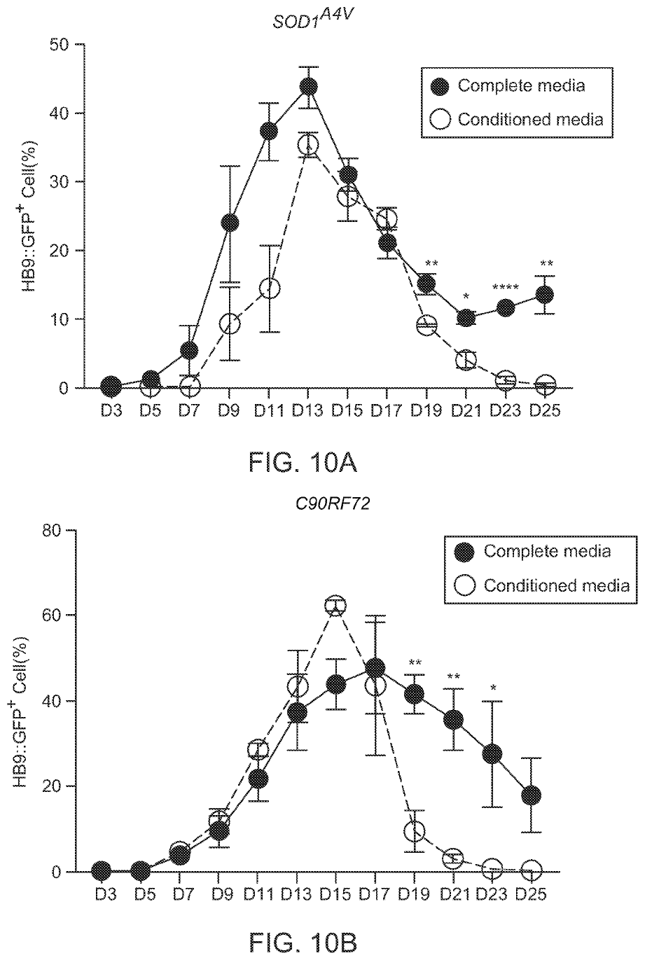

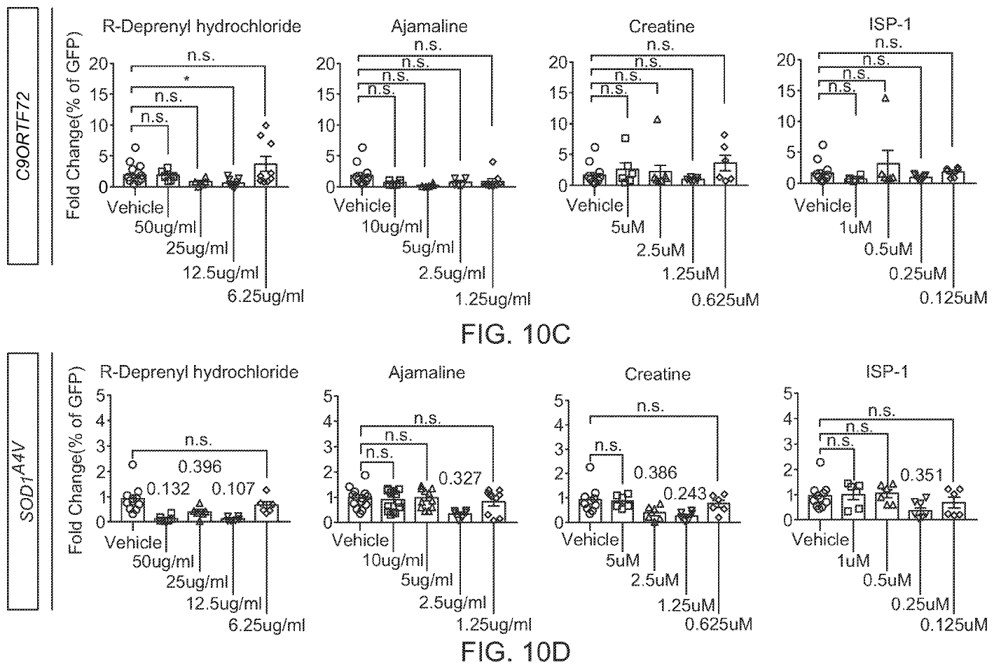

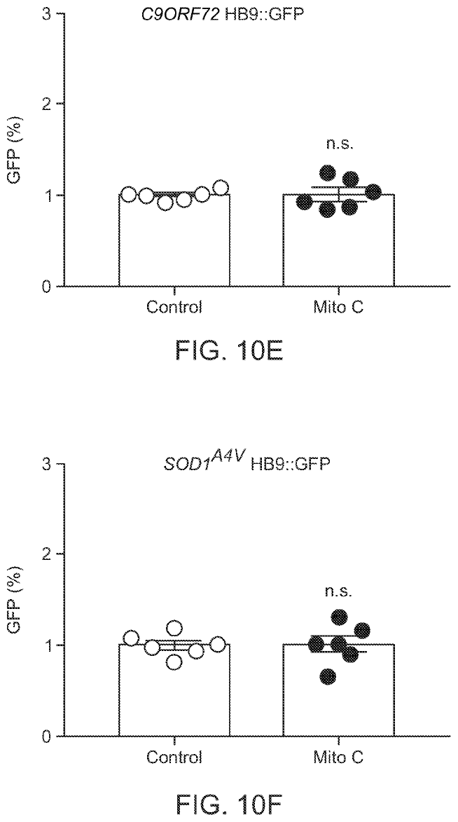

It is well known that the cellular level of AA is critical to cell viability and linked to disease phenotypes such as hypertension, cancer and leukemia⁷⁵⁻⁷⁷. To examine the functional contribution of AA metabolism to sMN pathology, a set of functional analogs of AA861(C21H26O3), a known 5-LOX inhibitor⁷⁸⁻⁸⁰, were tested including caffeic acid (CA: 3,4-dihydroxy benenearcrylic acid), apigenin, BW755C and nordihydroguaretic acid in optimized condition (FIGS. 10A-B). Treatment of those compounds at day 11 for 12 consecutive days (until day 23, during sMN differentiation protocol, FIG. 5A) was mostly sufficient to increase the numbers of HB9::GFP⁺ cells in SOD1^{A4V} and C9ORF72 ALS lines (FIG. 5B-C) (detailed information of fold change analysis is in methods). In addition, because the AA level seems to be critical for HB9::GFP⁺ sMNs survival, it was tested whether direct modulation of AA levels affects the levels of HB9::GFP⁺ cells. The AA treatment in differentiating HB9::GFP C9ORF72 and SOD1^{A4V} ALS lines decreased the percentages of HB9::GFP⁺ cells, and increased the percentages of 7AAD⁺ (cell death marker) cells, which were significantly reversed by CA treatment in a dose dependent manner. These data demonstrate that up-regulated AA level negatively affects viability of HB9::GFP⁺ sMNs, and a possible direct interaction between AA and CA in vitro (FIGS. 5D-E and FIGS. 10E-H).

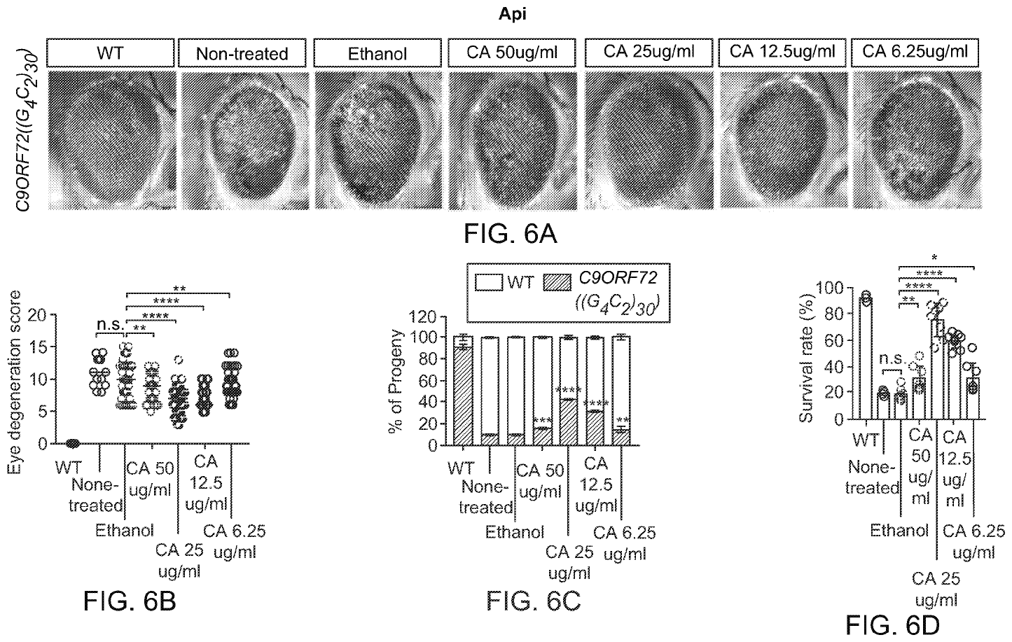

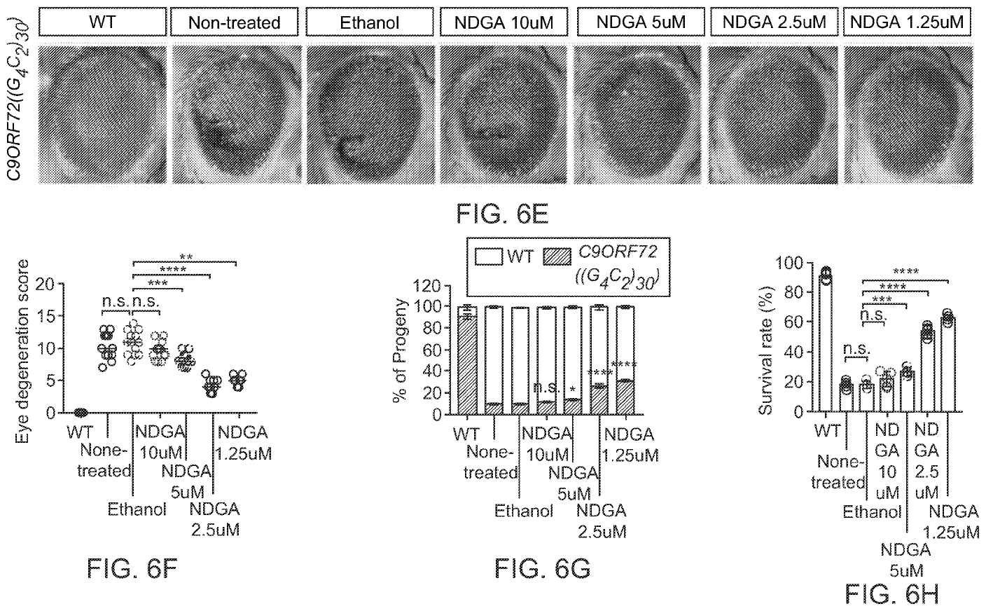

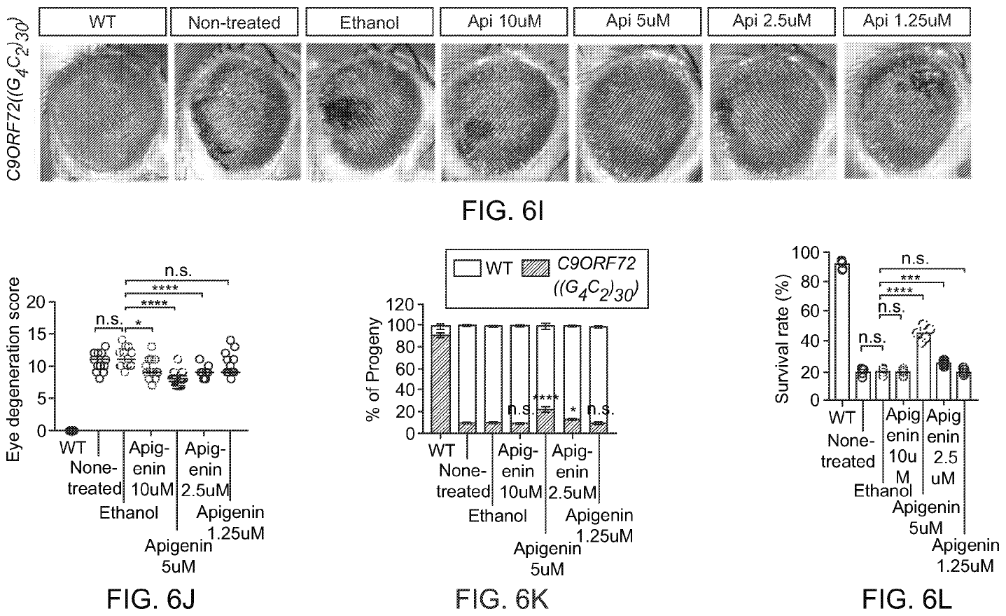

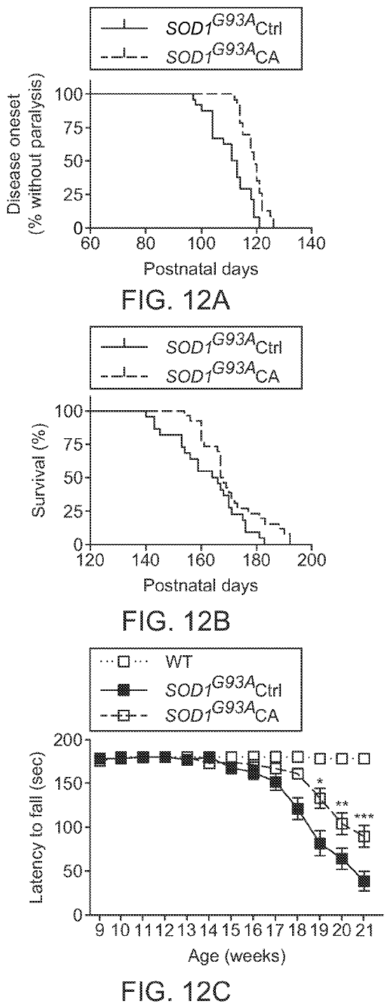

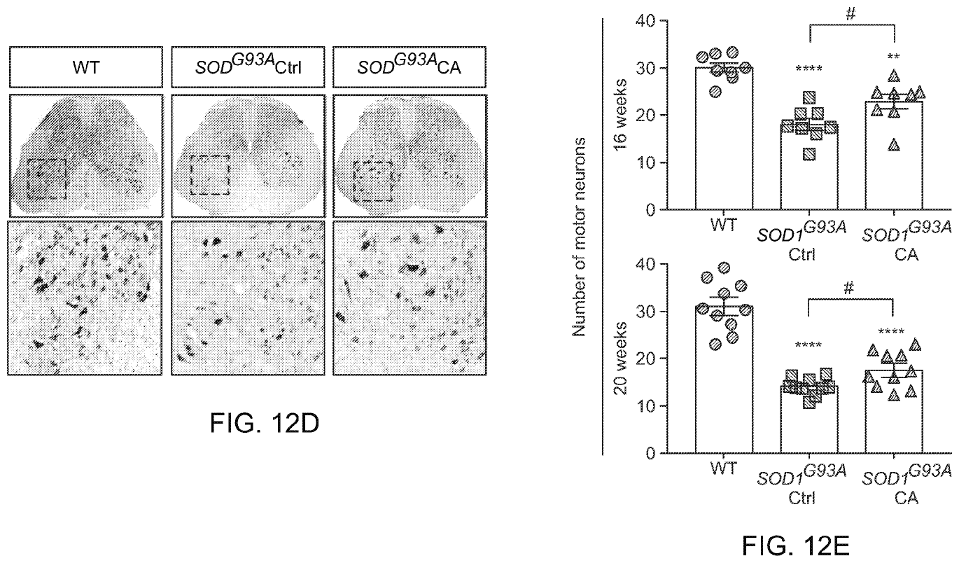

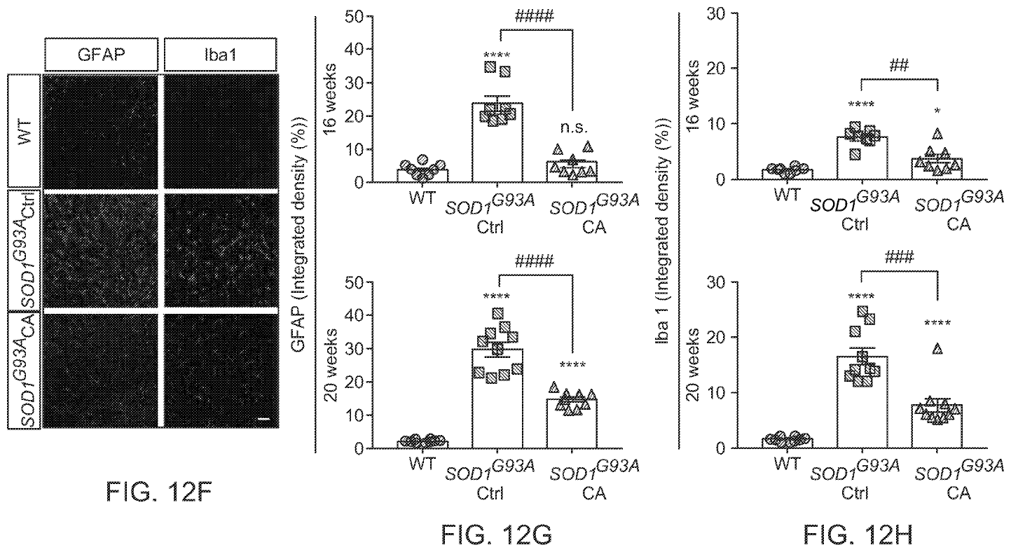

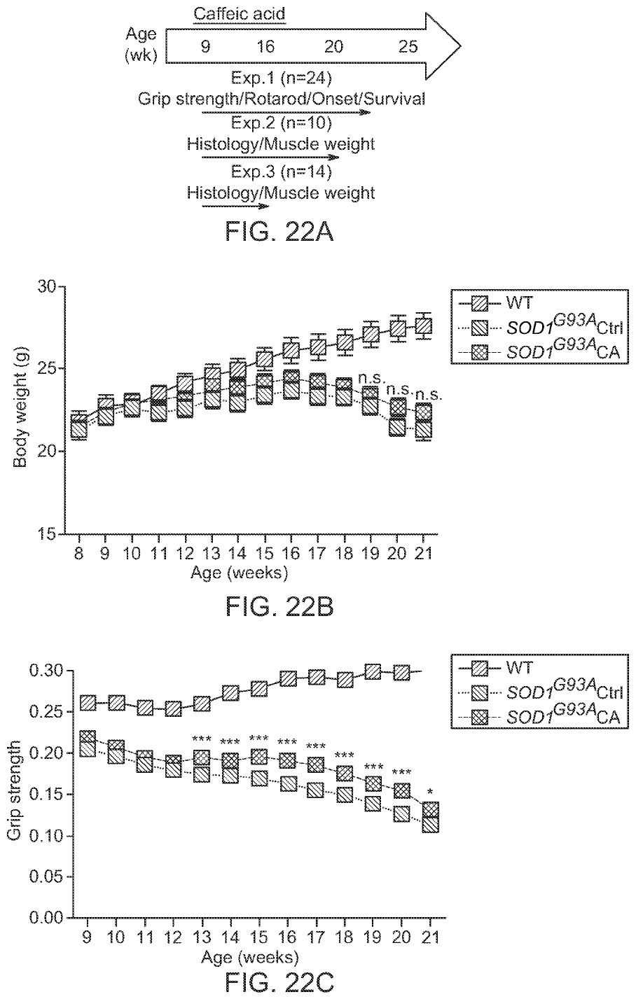

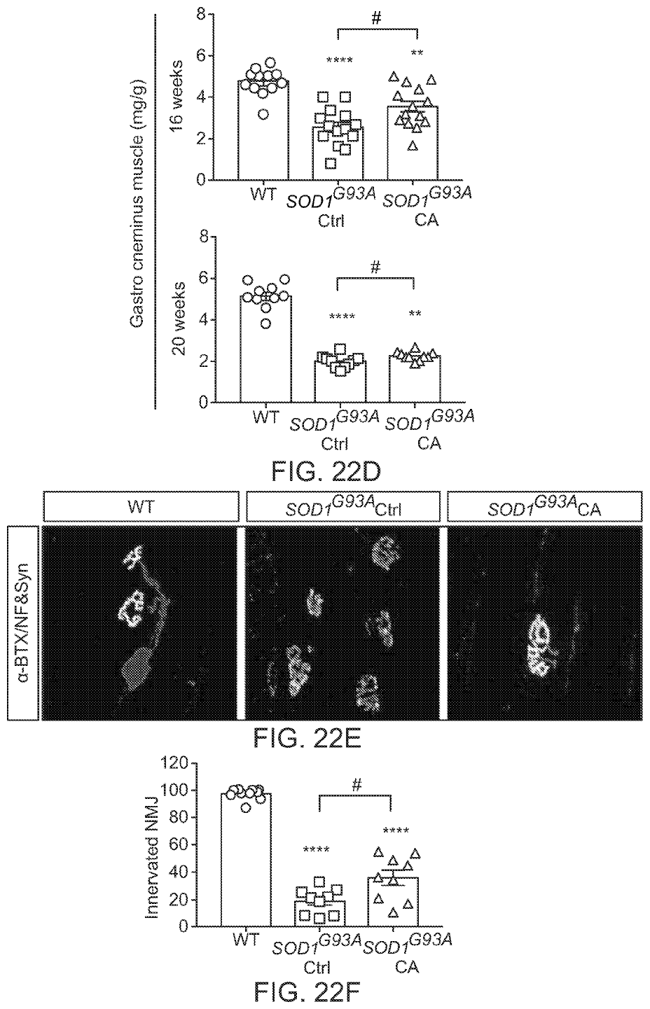

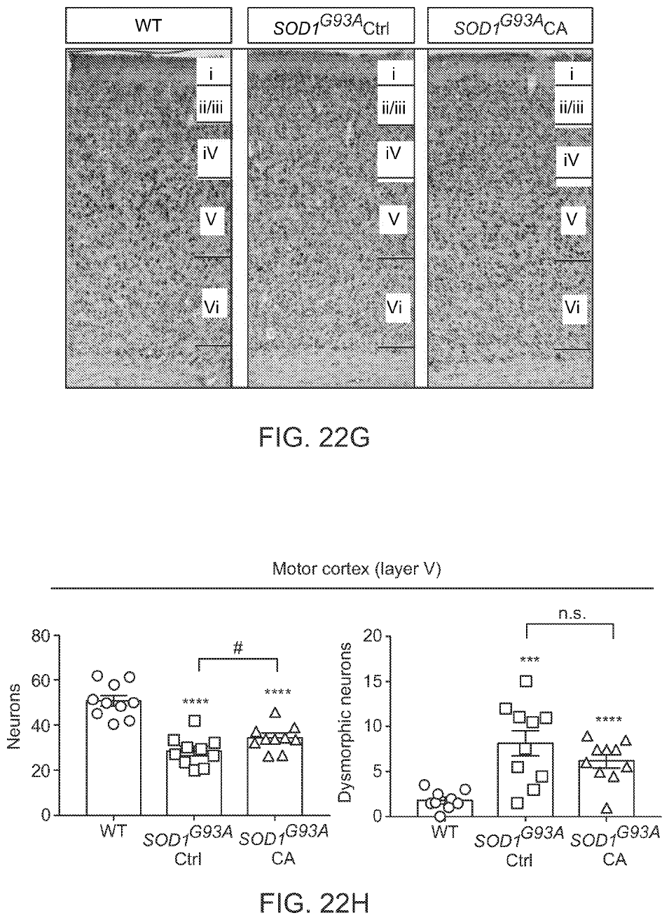

To confirm the rescuing effects of CA treatment seen in the in vitro hESC and hiPSC sMN model, a Drosophila model of C9ORF72-ALS that overexpresses 30 G4C2 repeats⁸¹ (SEQ ID NO:82) was employed. Surprisingly, significant dose-dependent recovery of eye degeneration phenotypes, progeny rate and survival rate were observed in this model CA (Caffeic acid), NDGA (Nordihydroguaretic acid) and Api. (Apigenin) (FIG. 6A-L). It was investigated whether caffeic acid ameliorates ALS phenotype in a mouse model. The efficacy was evaluated with three independent experiments where caffeic acid was administered to SOD1^{G934} mice from 60 days to 120 days of age (FIG. 22). The first experiment (Exp 1) was for the assessment of the disease onset, survival, and behavioral test, and the other experiments (Exp 2 and Exp 3) were for histological analyses. In the Exp 1 (n=24 for each group), caffeic acid was found to delay the disease onset and survival (FIGS. 6-A). The disease onset, determined by tremor and hind-limb splay defects, was significantly delayed in caffeic acid administered group (118.8±4.3 days) compared to control SOD1^{G93A} mice (109.8±7.7 days) (FIG. 12A). The delay of disease onset was also correlated with the lifespan of the mice. The survival of SOD 1^{G934} mice, determined by loss of righting reflex within 30s, was also significantly extended in caffeic acid administered mice (171.0±11.4 days) compared to control mice (162.8±12.3 days) (FIG. 12B). The attenuated disease symptom was also observed in locomotor performance. SOD1^{G93A} mice began to rapid reduction in rotarod performance from 15 weeks of age and, however, caffeic acid administration result in significant slowdown of the reduction (FIG. 12C). The attenuated disease progression by caffeic acid was also observed in body weight and grip strength (FIG. 22).

Next, the integrity of motor neuron and astrogliosis was analyzed using histological assessments at early symptomatic stage (16 wks, Exp 2) and late symptomatic stage (20 wks, Exp 3). The number of motor neuron (larger than 25 μm) in the ventral horn of spinal cord was significantly reduced in control SOD1^{G93A} mice (18.0±3.5) compared to wild-type mice (30.0±2.8) at early symptomatic stage, however, the number was significantly increased by the administration of caffeic acid (22.8±4.3). The significant difference was also maintained at late symptomatic stage (13.9±1.8 in control vs 17.5±3.8 in caffeic acid) (FIGS. 12D and E). Fluorescent staining with GFAP and Iba1 antibodies revealed that SOD1^{G93A} mice exhibit increased numbers of activated astrocytes and microglia in the spinal cord compared to wild-type mice. On the other hand, caffeic acid administration attenuated their activation (FIGS. 12F and G). In addition, innervated neuromuscular junction was also significantly spared in caffeic acid treated mice and the weight of gastrocnemius muscle was correlated with the attenuated disease symptom (FIG. 22). Collectively, these data suggested that CA-mediated pharmacological modulation of the AA pathway has a potential for therapeutic benefit for multiple ALS models.

Next, it was tested how dysregulation of the AA pathway mediates ALS pathogenesis using CA treatment, an inhibitor of 5-lipoxygenase (5-LOX)^{73,74}, can restore the dysregulated levels of AA and related glycophopholipid species. Levels of AA and glycophospholipids were quantified before and after CA treatment in sMN culture of the 4 ALS lines using a focused metabolomics analysis. When comparing vehicle (ethanol) and CA (25 μg/ml) treated ALS samples (C9ORF72, 6 lines: SOD1, 3 lines: TDP43, 3 lines: sporadic, 5 lines sMN culture), significantly decreased levels of AA and increased levels of glycophospholipid species in CA-treated ALS samples were observed (FIGS. 23A-F). Collectively, CA treatment reverses ALS-related phenotypes through the metabolic modulation of AA, suggesting that AA metabolism might be a rich source of promising drug targets for multiple ALS cases, and CA serves as a chemical scaffold of AA inhibitors (FIG. 23G).

FIGS. 5A-5E are data graphs showing that 5-LOX inhibitors rescue motor neuron degeneration in vitro according to an embodiment. (FIG. 5A) Schematic timeline of compounds treatment during sMN differentiation. (FIGS. 5B-5C) Administration of 5-LOX inhibitors (Caffeic acid, Apigenin, BW755C and Nordihydroguaretic acid) in C9ORF72 (B) and SOD1^{A4V} (Caffeic acid, Apigenin and Nordihydroguaretic acid) (FIG. 5C) sufficiently rescue the reduced levels of HB9::GFP⁺ cells (D11-D23, *P<0.05, **P<0.01, ****P<0.0001, n.s.: not significant, unpaired student's t-test, each dot indicates individual wells from at least 3 experimental repeats/batches). (FIGS. 5D-5E) Treated AA in sMN culture reduced the percentages of HB9::GFP⁺ cells and induced the percentages of 7AAD⁺ cells of SOD1^{A4V} and C9ORF72 by FACS analysis (FIG. 5D), but CA treatment reversed the levels of HB9::GFP⁺ cells and 7AAD⁺ cell (FIG. 5E) (complete media: n=2 for each ALS lines) (n.s.: not significant, *P<0.05, **P<0.01, ****P<0.0001, unpaired student's t-test). Error bars: mean±SEM.

FIGS. 6A-6J are images and data graphs showing that 5-LOX inhibitors rescue the phenotype of Drosophila model. Compounds rescue eye degeneration in C9ORF72(G₄C₂)₃₀) Drosophila model (“(G₄C₂)₃₀” disclosed as SEQ ID NO: 82) in a dose-dependent manner (CA (caffeic acid, FIGS. 6A-B) (; 6.25 μl to 50 μl, NDGA (nordihydroguaiaretic acid, FIGS. 6E-F): 1.25 μM to 5 μM, Api (apigenin, FIGS. 6I-J): 2.5 μM to 5 μM; at least n=13 for each group, n.s.: not significant, *P<0.05, **P<0.01, ***P<0.001 ****P<0.0001, unpaired student's t-test). (FIGS. 6C-D, 6G-H, 6K-L) Progeny efficiency and survival rate of Drosophila are also rescued by each compound feeding (n=5 for each group, n.s.: not significant, *P<0.05, **P<0.01, ***P<0.001 ****P<0.0001, unpaired student's t-test). Error bars: mean±SEM.

FIGS. 10A-10D are data graphs showing that caffeic acid exclusively rescues HB9::GFP⁺ cells in SOD 14 and C9ORF72 according to an embodiment. (FIGS. 10A-10B) Experimental modification of media (conditioned media) shows enhanced HB9::GFP⁺ degeneration of SOD1^{A4V} and C9ORF72 at D19 to D25 (complete media: n=3, conditioned media: n=4 for each ALS lines) (*P<0.05, **P<0.01, ****P<0.0001, unpaired student's t-test). (FIGS. 10C-10D) Results of compound tests in SOD1^{A4V} and C9ORF72 HB9::GFP⁺ cells indicate comparable effects to control vehicle for each compound (R-Deprenyl hydrochloride, Ajamaline, Creatine and ISP-1) by FACS analysis (Dot indicates different wells from at least 3 batches, at least n=6; technical replicates, n.s.: not significant, unpaired student's t-test). (FIGS. 10E-F) FACS results of HB9::GFP⁺ between control (non-treated) and mitomycin C treated (1 μg/ml, 1 hr: D17 to D19 (2 days)) in C9ORF72 and SOD1^{A4V} ALS hiPSC lines (n=6 for each group: technical replicates: n.s.: not significant: unpaired student's t-test). (FIGS. 19G-H) CA elevates the levels of HB9::GFP expression in the sMN culture of C9ORF72 and SOD1^{A4V} ALS hiPSC lines after mitomycin C treatment (Dot indicates different wells: technical replicates; n.s.: not significant: unpaired student's t-test). (Dot indicates different wells, n=3 for each group: technical replicates: n.s.: not significant; *, p<0.05: **, p<0.01: unpaired student's t-test). Error bars: mean±SEM.

FIGS. 12A-H show that Caffeic acid alleviates disease pathogenesis in SOD1^{G93A} mice according to an embodiment. FIGS. 12A-B are Kaplan-Meier curves of disease onset (A) and mice survival (B) in SOD1^{G93A} mice. (FIG. 12C) Locomotor performance evaluated by the rotarod test (n=24 for each group). FIG. 12D The motor neuron in L4-L5 segments of the spinal cord is visualized with Cresyl violet staining (Nissl staining) at early symptomatic stage (16wks) (n=10 for each group). FIG. 12E shows the number of motor neuron in L4-L5 segments of the spinal cord at 16 and 20 weeks. (F) Activated astrocytes (GFAP) and microglia (Iba1) in L4-L5 segments of the spinal cord at early symptomatic stage (16wks). (G) The integrated density of fraction area stained with GFAP at the indicated time points. (H) The integrated density of fraction area stained with Iba1. (WT: wild-type mice: Ctrl: vehicle administered SOD1^{G93A} mice; CA: caffeic acid (30 mg/kg) administered SOD1^{G93A} mice; *, #, p<0.05; **, p<0.01; ***, p<0.001 and ****, p<0.0001, n.s.: not significant, unpaired student's t-test). Scale bar (D) Error bars: mean±SEM.

FIGS. 22A-22H are images, data graphs, and schematics showing that Caffeic acid alleviates disease pathogenesis in SOD1^{G93A} mice according to an embodiment. (FIG. 22 A is an experimental scheme illustrating the caffeic acid administration and assessment of the efficacy. Caffeic acid or vehicle (PBS with 10% ethanol) was administered to SOD1^{G93A} mice from 60 days to 120 days of age (5 days per week). (FIG. 22B) Changes of body weight monitored weekly. (FIG. 22C) Grip strength analysis. n=24 for each group. (FIG. 22D) The ratio of gastrocnemius muscle to body weight (mg/g) at the indicated time points. n=14 at 16 wks and n=10 at 20 wks for each group. (FIG. 22E) Neuromuscular junction visualized by a-bungarotoxin (a-BTX, green) and neurofilament H/synapsin (NF/Syn, red) in gastrocnemius muscle at 16 wks. (FIG. 22F) The ratio of innervated neuromuscular junction (NMJ). n=8 for each group. WT, wild-type mice: Ctrl, vehicle administered SOD1^{G93A} mice: CA, caffeic acid (30 mg/kg) administered SODIG934 mice; *, #, p<0.05; **, p<0.01; ***, p<0.001 and ****, p<0.0001. (n.s.: not significant, unpaired student's t-test). Scale bar. Error bars: mean±SEM.

FIGS. 23A-23F present a schematic model of the study and data graphs showing that caffeic acid rescues aberrant levels of arachidonic acid in the sMN culture of multiple ALS hiPC lines according to an embodiment. (FIGS. 23A-F) Ion count value shows arachidonic acid level is down-regulated in post-treatment with 25 μg/ml CA at D11 to D17 of sMN differentiation of control and CA treated of ALS hiPSC lines (at least n=3 for each group: the cell line names are listed: technical replicates: biological replicates: *P<0.05, **P<0.01, ***P<0.001, unpaired student's t-test). (FIG. 23G) Schematic model of this study. Error bars: mean±SEM.

### DISCUSSION

Here, through unbiased comparative multi-omics analyses of two distinct motor neuron subtypes (oMN-like PHOX2B::GFP⁺ and HB9::GFP⁺ sMNs), it was determined that various pathways in lipid metabolism, especially the AA metabolic pathway, are dysregulated in SOD1^{A4V} and mutant (9ORF72 HB9::GFP⁺ populations but normal in PHOX2B::GFP⁺ populations. These findings were also validated by a targeted metabolomics study between healthy and ALS (SOD1^{A4V}, C9ORF72. TDP 439343R and sporadic) neuronal populations. Importantly, the ALS phenotypes are rescuable, both in vitro and in vivo, by chemical regulation of the AA metabolism, showing an untapped translational potential of findings described in this study.

Previously, the several groups suggested therapeutic target molecules^{35,46,82}, but these transcriptome analysis results did not confirm those findings (data not shown). One possible reason is species and/or mutation differences between the SOD1^{G93A} mouse strain and the SOD1^{A4V} hiPSC-derived sMNs. For example, one previous study identified MMP-9 gene in a comparison of oMNs and sMNs in WT mice³⁵, but the comparative analysis was conducted on ALS-specific human oMN-like and sMNs in SOD1^{A4V} and C9ORF72 mutations. The comparative analysis between human oMN-like PHOX2B::GFP⁺ and HB9::GFP⁺ sMNs revealed significant changes in lipid metabolism pathways in ALS sMN populations. Interestingly, the expression levels of DEGs within the lipid metabolism pathways (FIG. 19) were mostly comparable in healthy oMN-like and sMNs, suggesting that the aberrant transcriptional levels in lipid related pathways is unique to the ALS background. Furthermore, it was attempted to pinpoint specific pathways using transcript profile by incorporating metabolomics analysis and assembling puzzle pieces. Targeted metabolomics of four ALS (SOD1^{A4V}, C9ORF72. TDP43^{Q343R} mutations and a sporadic hiPSC lines)-derived sMN differentiation was independently conducted to confirm the unbiased multi-omics results. As a result, it was confirmed that significant numbers of highly enriched (29 metabolites) or low level of metabolites (22 metabolites) common in four ALS (C9ORF72, 6 lines: SOD1, 3 lines: TDP43, 3 lines: sporadic, 5 lines) hiPSC-derived sMN cultures. Interestingly, the results of metabolomics and multi-omics not only consistently showed lipid metabolism, but also the pentose phosphate pathway (PPP) as well as histidine metabolism (FIGS. 3C and D and FIG. 4D) and purine/pyrimidine metabolism (FIGS. 3D and 4B) are dysregulated. Aberration of PPP together with nucleotide metabolism might be a metabolic signature of higher burden of DNA damage due to higher ROS level and redox imbalance^{83,84} in ALS sMN compared to healthy-derived sMN or ALS-derived oMN, which is corroborated by previous ALS studies^{22,34,85}. Taken together, the data clearly show that there is aberrant lipid homeostasis in sMN cultures of ALS hiPSCs and also imply that dysregulated lipid metabolic pathways might serve as therapeutic target for ALS patients.

Consistent with these findings, accumulating evidence has also shown potential connections between ALS pathogenesis and aberrant lipid mechanisms^{44-46,86}, but there are few proven detailed mechanistic studies. The present study is the first report providing systematic profiling of ALS patient motor neuron cells by recapitulating both SOD1^{A4V} and C9ORF72 HB9::GFP⁺ sMN versus PHOX2B::GFP⁺ oMN-like cells and reveal the causative contribution of lipid metabolism dysregulation to ALS pathogenesis by employing RNA sequencing and metabolomics analysis. These data were also consistent with another set of analysis for targeted metabolomics between healthy donor and ALS (SOD1^{A4V}, C9ORF72, TDP43^{Q343R} and sporadic) patient-derived sMN culture. Based on the multi-omics data analysis, one of the aberrantly regulated lipid metabolism pathways in ALS-derived sMN populations is the AA pathway. It was also identified that C21H26O3, an AA861 structural analog, is almost undetectable in sMN of ALS hiPSC lines (SOD1^{A4V}, C9ORF72. TDP43^{Q343R} and sporadic) by metabolomics analysis. Indeed, AA861 is a well-known natural inhibitor of 5-lipoxygenase (5-LOX) that metabolizes AA into other metabolites, which is consistent with the multi-omics data. Interestingly, AA levels are closely associated with apoptosis, suggesting that metabolic pathways regulating AA levels might be a therapeutic target for ALS^{75,76,79}. For example, other group showed that AA modulation by PLA2 (phospholipase A2) inhibitor has a protective effect in SOD1 mouse model (ref, Ouchi). Therefore, it was hypothesized that pharmacological modulation of the AA pathway could restore the levels of AA as well as ALS-relevant phenotypes. Indeed, treatment of several 5-LOX inhibitors was sufficient to restore the decreased levels of ALS-derived HB9::GFP⁺ cells (FIGS. 5B-C), and eye degeneration phenotypes and survival rate in the Drosophila model (FIGS. 6A-L) and SOD1^{G93A} mouse (FIG. 7). Considering the fact that CA treatment significantly decreased levels of AA and increased levels of phospholipid species in confirmatory metabolomics analysis (FIG. 6E) and AA-induced cell death was rescued by CA treatment in a dose-dependent manner in the ALS sMN cultures (FIGS. 5D-E), it is clear that inhibition of 5-LOX activity can tune down the levels of AA in ALS-derived sMN culture. However, at this moment, it is not clear how the lower levels of AA is linked to the increased levels of phospholipid species, which might be explained by Lands cycle^{74,87}.

Previously, another group reported that increased levels of ceramide were identified in CSF of ALS patients, and pharmacological inhibition of the sphingolipid synthesis pathway by ISP-1 could inhibit spinal motor neuron death in vitro⁴⁴. To find relevance of other lipid pathways in this study, two additional candidate metabolites were tested that are not detected in the four ALS line derived sMNs (Ajamaline and Creatine) and two chemical compounds that can compensate for altered levels of metabolites (R-Deprenyl hydrochloride and ISP-1 for decreasing the dys-regulated levels of Putrescine and Ceramide (d18:1/16:0), respectively) (FIGS. 9A-9K and FIGS. 10C-10D). Furthermore, several studies by MS analysis also demonstrated that they identified phosphatidylcholine (36:4) (ref, Blasco et al) and cholesteryl esters (ref, Chaves-Filho et al). However, no phenotypic rescuing effects were seen in the ALS-derived HB9::GFP⁺ sMN survival assay (FIG. 10C-10D). These data indicate that the AA pathway might play a pivotal role in ALS disease progression, at least in hiPSC-derived sMNs. In addition, another study focused on TNFa alteration in microglia cells and neuroinflammation showed pharmacological modulation of the AA pathway (by nordihydroguaiaretic acid), and improved survival rate of SOD1^{G93A} mouse⁸⁸. Collectively, the studies discussed herein provide extensive targeted metabolomics profiles of ALS sMN culture and identified the commonly present or undetectable metabolites as potential therapeutic targets, as shown in the example with AA861/CA.

Taken together, the data herein demonstrate that substantially dysregulated lipid metabolism pathways are common in 17 different ALS hiPSC-derived sMN cultures, and pharmacological modulation of AA metabolism shows protective effects in an in vitro human sMN model and a Drosophila and SOD1^{G93A} mouse model. The current study provides a new framework for disease modeling by comparing affected and non-affected cell types from a disease hiPSC line, leading to the unraveling of metabolic aberrations in ALS sMN and identification of potential drug candidates.

### Methods

Generation of Reporter Lines in hESC/iPSC by CRISPR-Cas9)

CRISPR-Cas9 knock-in strategy was performed as previously described⁸⁹. Feeder-free H9 hESCs, 01582 hiPSCs (PHOX2B::GFP)⁵⁶, and C9ORF72 and SOD1^{A4V} iPSC lines (PHOX2B::GFP and HB9::GFP) were dissociated using Accutase (Innovative Cell Technologies Inc.). Cells (2×10⁶) were resuspended in nucleofection solution V (Lonza) with 4 μg of hCas9—gRNA plasmid (gRNA #1 and #2 were used for HB9::GFP) and 4 μg of dsDNA donor plasmid. The nucleofection was performed by Nucleofector™ II according to manufacturer's instruction (B-16, Lonza), then nucleofected cells were plated on puromycin resistant MEFs (DR4, Global Stem) in hES medium (DMEM/F12 (Invitrogen) containing 20% knockout serum replacement (KSR, Gibco), 0.1 mM MEM-NEAA (Gibco), 1 mM L-glutamine (Gibco), 55 uM β-mercaptoethanol (Gibco), 4 ng/ml FGF2 (Gibco)) with 10 μM Y-27632 (Cayman Chemical). After 3 or 4 days, knock-in cells were selected by treatment with 0.5 μg/ml puromycin (MilliporeSigma) in hES medium. After selection, puromycin resistant colonies were verified for GFP expression by FACS analysis using each differentiation protocol.

### Plasmid Constructions

For the PHOX2B::GFP reporter line, plasmids were used as previously described⁵⁶. For the HB9::GFP reporter line, left arm 1512 bp and right arm 900 bp were designed from stop codon of the human HB9 locus. Each arm was generated by PCR using (H9) hESC genomic DNA and inserted into OCT4-2A-eGFP-PGK-Puro donor vector backbone (Addgene #31938)⁹⁰ between BamHI and NheI for left arm and AscI and NotI for right arm. The gRNA sequence was designed by Zhang lab gRNA design resource⁸⁹ and subcloned into gRNA vector (Addgene #48138) as previously described⁹¹. All insert sequences were verified by DNA sequencing (JHU synthesis & sequencing facility).

HB9 left arm
(SEQ ID NO: 1)
F: ATAGGATCCTCAACTCCTGGGCTTCCCGGAACCT
(SEQ ID NO: 2)
R: ATAGCTAGCCTGGGGCGCGGGCTGGTGGCTGGGC
right arm
(SEQ ID NO: 3)
F: ATAGGCGCGCCGAGCCCCGCGCCCAGCAGGTGCGGC
(SEQ ID NO: 4)
R: ATAGCGGCCGCCCCGGGACAGGTGTGCACCAGGCAG
gRNA#1
(SEQ ID NO: 5)
F: CACCGTACAGCAACGGCGCCAGCGT
(SEQ ID NO: 6)
R: AAACACGCTGGCGCCGTTGCTGTAC
gRNA#2
(SEQ ID NO: 7)
F: CACCGCGGAGGACGACTCGCCGCCC
(SEQ ID NO: 8)
R: AAACGGGCGGCGAGTCGTCCTCCGC

hESC/iPSC Culture and Differentiation

H9 hESCs, 01582 (GM01582), 2623 (GM02623), 24C (GM00024C) iPSCs (derived from each fibroblasts, Coriell Institute) and OCT4::GFP in hESC (H9), PHOX2B::GFP in hESC (H9) and 01582 iPSC, C9ORF72, SOD1^{A4V} for PHOX2B::GFP and HB9::GFP (ALS patient fibroblasts (JH078[C9ORF72]⁹² and GO013 [SOD1^{A4V}]⁹³ were collected at Johns Hopkins hospital with patient consent)⁹⁴. TDP43^{Q343R} (gift from Nicolas J. Maragakis), Sporadic (gift from Nicolas J. Maragakis) iPSC were cultured (passages 12-60) on inactivated mouse embryonic fibroblasts (MEF, Applied Stem Cell) with hES medium at 37° C. and 5% CO₂ in a humidified incubator as described previously 18. For neuronal differentiation, LSB (LDN193189+SB431542) protocol was used as described previously^{95,96} and adapted for each neuronal differentiation.

### FACS Analysis and Sorting

Cells were dissociated using Accutase after incubation for 20 min at 37° C. and suspended with buffer containing 40 μg/ml DNase I (Roche Applied Science). For GFP analysis, BD FACS Calibur (Becton Dickinson) and FlowJo software (Tree Star Inc.) were used. To purify GFP⁺ populations for each reporter line, a MoFlo high-speed sorter (Dako Cytomation) in the Johns Hopkins School of Public Health Flow Cytometry Core Facility and a BD FACSJazz sorter in the Stem Cell Core Facility of Johns Hopkins Medicine (Institute for Cell Engineering) were used.

### Immunofluorescence Staining

For Immunohistochemistry, E12.5 midbrain was dissected and fixed with 4% paraformaldehyde (PFA) overnight. After fixation, tissues were washed with PBS and incubated with 30% sucrose for cryosection as described previously⁹⁷. The following antibodies were used as a primary antibody: rabbit anti-TH (Pel-Freez Biologicals), mouse anti-Isl1 (DSHB) and rabbit anti-Phox2b (gift from Jean-Francois Brunet)⁹⁸. For Isl1 staining, a mouse on mouse kit (Vector Laboratory) was used. For immunocytochemistry, cells were fixed with 4% PFA and stained with mouse anti-ISL1 (DSHB), mouse anti-NKX6.1 (DSHB), rabbit anti-TUJ1 (BioLegend) and rabbit anti-PHOX2B (gift from Jean-Francois Brunet). For FACS analysis, cells were fixed with 4% PFA and stained with mouse anti-HB9 (DSHB) and mouse anti-ISL1 (DSHB) as described previously⁹⁹. For 7AAD analysis by FACS, staining was conducted based on commercial instruction (BD #559925). Appropriate Alexa Fluor 488, 568 and 647 (Life Technologies) labeled secondary antibodies were used with DAPI (Roche Applied Science) for nuclear staining. All images were visualized with fluorescence microscopy (Eclipse TE2000-E, Nikon).

qRT-PCR Analysis and Primer Information

Total RNA was extracted using TRIzol Reagent (Life Technologies) and reverse transcribed using High Capacity cDNA Reverse Transcription kit (Applied Biosystem). qRT-PCR was performed using SYBR Green PCR Master Mix (Applied Biosystems) and Mastercycler ep Realplex2 (Eppendorf) with the primers shown below. All primers were designed using GenScript primer design software.

| Gene | Forward | Reverse |
|---|---|---|
| GAPDH | TGCACCACCAACTGCTTAGC | GGCATGGACTGTGGTCATGAG |
| | (SEQ ID NO: 9) | (SEQ ID NO: 10) |
| OCT4 | AGTGAGAGGCAACCTGGAGA | ACACTCGGACCACATCCTTC |
| | (SEQ ID NO: 11) | (SEQ ID NO: 12) |
| NANOG | CATGAGTGTGGATCCAGCTT | CCTGAATAAGCAGATCCATG |
| | (SEQ ID NO: 13) | (SEQ ID NO: 14) |
| ISL1 | GGTTTCTCCGGATTTGGAAT | CACGAAGTCGTTCTTGCTGA |
| | (SEQ ID NO: 15) | (SEQ ID NO: 16) |
| EN1 | TCTCGCTGTCTCTCCCTCTC | CGTGGCTTACTCCCCATTTA |
| | (SEQ ID NO: 17) | (SEQ ID NO: 18) |
| PHOX2A | CCGATGGACTACTCCTACCTCA | GCAGGGGGCTGTATTGGAAG |
| | (SEQ ID NO: 19) | (SEQ ID NO: 20) |
| GATA2 | CGGTCCTGCACAGATTCCCA | CAGCAGCTTCGGCCTCAAAG |
| | (SEQ ID NO: 21) | (SEQ ID NO: 22) |
| GATA3 | CGGAGGAGGTGGATGTGCTT | GCCCTGACCGAGTTTCCGTA |
| | (SEQ ID NO: 23) | (SEQ ID NO: 24) |
| NURR1 | CAAGTCACATGGGCAGAGATAG | GGCTAGGAGGGTTACAGAAATG |
| | (SEQ ID NO: 25) | (SEQ ID NO: 26) |
| HOXA2 | ACAAGTACCTTTGCAGACCC | CATTTCCCTTCGCTGTTTTGG |
| | (SEQ ID NO: 27) | (SEQ ID NO: 28) |
| HOXB2 | TTTAGCCGTTCGCTTAGAGG | CGGATAGCTGGAGACAGGAG |
| | (SEQ ID NO: 29) | (SEQ ID NO: 30) |
| HOXA5 | GGCCTTCCGTCCCTGAGTAT | GCAACGAGAACAGGGCTTCT |
| | (SEQ ID NO: 31) | (SEQ ID NO: 32) |
| HOXA7 | TCTCCCTCCTCTGTCACCCT | CCCGACCCTCTGTCCTCATT |
| | (SEQ ID NO: 33) | (SEQ ID NO: 34) |
| HOXA10 | GCCTGAGGTCAATGGTGCAA | AAAGTCAAGCCCGTTTGCCA |
| | (SEQ ID NO: 35) | (SEQ ID NO: 36) |
| HB9 | GCACCAGTTCAAGCTCAAC | GCTGCGTTTCCATTTCATCC |
| | (SEQ ID NO: 37) | (SEQ ID NO: 38) |
| FOXP1 | AGACAAAAAGTAACGGTTCAGCC | CGCACTCTAGTAAGTGGTTGC |
| | (SEQ ID NO: 39) | (SEQ ID NO: 40) |
| LHX3 | GCTGGGCCCGGGAAAGTTCG | GTGCTAGCAGCAGGTCGCCTC |
| | (SEQ ID NO: 41) | (SEQ ID NO: 42) |
| NKX6.1 | GAGATGAAGACCCCGCTGTA | GACGACGACGAGGACGAG |
| | (SEQ ID NO: 43) | (SEQ ID NO: 44) |
| MAP2 | CAGGAGACAGAGATGAGAATTCC | CAGGAGTGATGGCAGTAGAC |
| | (SEQ ID NO: 45) | (SEQ ID NO: 46) |
| CHAT | GACGTCTGACGGGAGGAG | TCAATCATGTCCAGCGAGTC |
| | (SEQ ID NO: 47) | (SEQ ID NO: 48) |
| ACSM1 | AGGAGGGCAAGAGAGGTCCA | ACCAGCCACCACTCAGGAAC |
| | (SEQ ID NO: 49) | (SEQ ID NO: 50) |
| PSAPL1 | CACTCATCCGCCACCAAAGC | CCTTGCTCCTCCTGCCTCTC |
| | (SEQ ID NO: 51) | (SEQ ID NO: 52) |
| SYNJ2 | GACAGACAGGGTGCTGTGGT | TTGTAGCTCCGCACGACCAT |
| | (SEQ ID NO: 53) | (SEQ ID NO: 54) |
| TMEM30B | ATCCGCCAGGGCAACTACTC | CCACCCATCCACGAGATGCT |
| | (SEQ ID NO: 55) | (SEQ ID NO: 56) |
| ADAM8 | CCCACCCTTCCCAGTTCCTG | GGTGCGAACGTTGGCTTGAT |
| | (SEQ ID NO: 57) | (SEQ ID NO: 58) |
| ABCG1 | GCTTCCTCAGTCCAGTCGCT | CATGCTCGGACTCTCTGCCA |
| | (SEQ ID NO: 59) | (SEQ ID NO: 60) |
| PPARA | CAGAACAAGGAGGCGGAGGT | GTTTGCGAAGCCTGGGATGG |
| | (SEQ ID NO: 61) | (SEQ ID NO: 62) |
| SLC27A6 | CGCGCCACACTTCTCTAGGT | ACGAAACACGGTGGGAGTGT |
| | (SEQ ID NO: 63) | (SEQ ID NO: 64) |
| SLC27A2 | CGTGGCGCTCCTTATGGGTA | CACTGGAAGCAGTGCAGCAG |
| | (SEQ ID NO: 65) | (SEQ ID NO: 66) |
| PPARG | TGGTCTTGTCGGCAGGAGAC | CCCAAAGTTGGTGGGCCAGA |
| | (SEQ ID NO: 67) | (SEQ ID NO: 68) |
| LRAT | CCTGGCCTGCAGGATGAAGA | CCTCGGTGGAAAGAGCTGGT |
| | (SEQ ID NO: 69) | (SEQ ID NO: 70) |
| PLA2G10 | TGTGTGCCTGCCAATCATGC | ACAACCCACAGTTCCTGCCA |
| | (SEQ ID NO: 71) | (SEQ ID NO: 72) |
| APOA1 | AAGGCCACCGAGCATCTGAG | ATTCTGAGCACCGGGAAGGG |
| | (SEQ ID NO: 73) | (SEQ ID NO: 74) |
| GHRL | GCATGCTCTGGCTGGACTTG | GCTTGGCTGGTGGCTTCTTC |
| | (SEQ ID NO: 75) | (SEQ ID NO: 76) |
| CPTIA | GTCACCATGCGCTACTCCCT | GCAGCGATGTCTGGAAGCTG |
| | (SEQ ID NO: 77) | (SEQ ID NO: 78) |
| SOAT2 | CCTAGGCCCTGGGATGTGTG | GAAGGGCTCTCGGCTCATGT |
| | (SEQ ID NO: 79) | (SEQ ID NO: 80) |
| TBX20 | GGGAGGATGGTCACCTGAAA | CTGGCTGTGATGTCAGCTTC |
| | (SEQ ID NO: 83) | (SEQ ID NO: 84) |
| VACHT | GGCATAGCCCTAGTCGACAC | CGTAGGCCACCGAATAGGAG |
| | (SEQ ID NO: 85) | (SEQ ID NO: 86) |
| CASPASE3 | TATTCAGGCCTGCCGTGGTA | GGCACAAAGCGACTGGATGA |
| | (SEQ ID NO: 87) | (SEQ ID NO: 88) |

### Motor Neuron Compound Screening and Analysis

SMN differentiation was performed as previously described⁶⁷. For general sMN culture media, neurobasal medium (Gibco) containing B27 (Gibco), N2 (Gibco), and 2 mM L-glutamine was used as a normal medium. For compound testing, neurobasal medium with N2 was used as a conditioned medium using caffeic acid (Sigma, C0625), R-Deprenyl hydrochloride (Sigma, M003), Ajamaline (MP Biomedicals, 4360-12-7), Creatine (Sigma, 1150320) and ISP-1 (Sigma, M1177), BW755C (Tocris, 105910), Nordihydroguaiaretic acid (Sigma, 74540), Apigenin (Fisher Scientific, 50908414), U-73122 (Thermo, 126810). For Arachidonic acid testing, Arachidonic acid (Cayman, 506-32-1) was treated in normal media. For mitomycin C treatment, 1 μg/ml of mitomycin C was treated in differentiating oMN or sMN cells for 1 hr at D17 and analyzed after 2 days (D19) by FACS. For fold change value, non-treated % of GFP⁺ were considered as a control and fold change values were normalized upon % of GFP expression of non-treated cells by FACS. For fold change value, non-treated cells were considered as a control and fold change values were normalized upon GFP expression of non-treated cells by FACS.

### RNA Sequencing

Total RNA was extracted using miRNeasy mini kit (Qiagen) as per manufacturer's instructions. RNA concentration and purity were assessed by Nanodrop (Thermo Fisher), and RNA integrity was assessed using the Agilent Bioanalyzer. cDNA Libraries were prepared for mRNA-enriched sequencing using TruSeq Stranded mRNA kit (Illumina). This was followed by normalization to 6 nM and pooling of libraries, followed by single end 75 bp sequencing on the Illumina HighSeq 4000. RNA-seq reads were aligned to the human reference genome (gencode.v27.primary_assembly.annotation.gtf downloaded from Gencode) using STAR aligner¹⁰⁰. Differential gene expression analysis was performed using EdgeR¹⁰¹ and Limma-Voom¹⁰². The Limma-Voom package was used for data normalization and generation of differential expression gene matrices. Genes with |fold change (FC)|>=2 and adjusted p-value <0.05 were considered as differentially expressed genes: 4,016 up-regulated and 3,749 down-regulated DEGs were identified. To reduce the number of differentially expressed genes, the Treat method¹⁰³ was used to calculate p-values from empirical Bayes moderated t-statistics with minimum log-FC requirement. The number of differentially expressed genes was reduced to a total of 1,806 DEGs for comparison of HB9::GFP⁺ versus PHOX2B::GFP⁺. To identify enriched transcriptomic signatures out of the large number of DEGs between HB9::GFP⁺ and PHOX2B::GFP⁺, a non-parametric method was used, gene set enrichment analysis (GSEA), where the enrichment score reflects the degree to which a gene set is over-represented at the top or bottom of a ranked list of genes¹⁰⁴. Enrichment analysis and visualization were performed using clusterProfiler R package¹⁰⁵ and biological process terms from Gene Ontology (GO) with gene set size between 15 and 300 genes. To validate the oMN-like and sMN populations, the datasets were compared with previous mouse dataset from a previous literature where transcriptomic signature of oMN and sMN was well established⁶⁸ (GEO dataset id: GSE118620). The mouse dataset was aligned to mouse reference genome (gencode. vM24.primary_assembly.annotation.gtf downloaded from Gencode) and further analyzed in the same way as thehuman dataset is analyzed. For healthy control of hESC and hiPSC lines, oMN-like cells were differentiated from hESC/hiPSC PHOX2B::GFP lines, and sMN cells from hESC/hiPSC lines were stained by HB9 antibody (DSHB) (ref, Rubin). A pooled library of 12 samples was subjected to Illumina platform (NovaSeq6000 S4) in 150 bp paired-end mode. Raw data (FASTQ files) were imported into Altanalyze v2.1.4 software, which uses the embedded software Kallisto and Ensembl 72 annotations. Two QC-failed control samples were excluded from the analyses. Processed expression files, including transcript-level expression values (TPMs) summed at the gene-level and read counts, were used in R (v4.0.3) to generate volcano plot, principal component (PC) plot, heatmaps, and dot plots shown in FIG. 19. The voom function in the limma package (v3.46.0) was used to identify genes having |fold change (log 2-base)|>2 and adjusted p-value<0.05, which were considered differentially expressed (DE). The prcomp function in the stats package (v4.0.3) was used to perform PC analysis using DE genes in ALS sMN samples (HB9::GFP⁺ cells of C9ORF72 and SOD1^{A4V}). The heatmap function in the heatmap package (v1.0.12) was used to generate heatmaps which clustered rows and columns (Pearson correlations). The enrichr function in the enrichR package (v3.0) was used to perform enrichment analysis of up-regulated gene sets using GO Biological Process (2018) database.

### LC-MS Metabolomics

Liquid chromatography mass spectrometry (LC-MS) differentiation and detection of each metabolite (C9ORF72 PHOX2B::GFP⁺, SOD1^{A4V} PHOX2B::GFP⁺, C9ORF72 HB9::GFP⁺, SOD1^{A4V} HB9::GFP⁺, un-sorting of C9ORF72, SOD1^{A4V}, TDP43^{Q343R}, Sporadic and control line derived sMN) were performed with an Agilent Accurate Mass 6230 TOF coupled with an Agilent 1290 Liquid Chromatography system using a Cogent Diamond Hydride Type C column (Microsolve Technologies, Long Branch, NJ, USA) with solvents and configuration as previously described¹⁰⁶. An isocratic pump was used for continuous infusion of a reference mass solution to allow mass axis calibration. Detected ions were classified as metabolites based on unique accurate mass-retention time identifiers for masses showing the expected distribution of accompanying isotopologues. Metabolites were analyzed using Agilent Qualitative Analysis B.07.00 and Profinder B.08.00 software (Agilent Technologies, Santa Clara, CA, USA) with a mass tolerance of <0.005 Da. Standards of authentic chemicals of known amounts were mixed with bacterial lysates and analyzed to generate the standard curves used to quantify metabolite levels. All data obtained by metabolomics profiling were the average of at least two independent triplicates. Bioinformatics analysis was carried out using MetaboAnalyst v.4.0 (www.metaboanalyst.ca), which is a web-based available software for processing metabolomics data, and pathway mapping was performed on the basis of annotated Human metabolic pathways available in the Kyoto Encyclopedia of Genes and Genomes pathway database. Metabolomics data were analyzed by statistical analysis. The clustered heat map and hierarchical clustering trees were generated using Cluster 3.0 and Java Tree View 1.0. A univariate statistical analysis involving an unpaired t-test was used to identify significant differences in the abundances of metabolites between each group.

### Transgenic Mice

Hb9::GFP and ISL1::GFP mice were described previously^{107,108}. All experiments used protocols approved by the Animal Care and Ethics Committees of the Gwangju Institute of Science and Technology (GIST) in accordance with the National Institutes of Health Guide for the Care and Use of Laboratory Animals. For efficacy assessment of the caffeic acid, B6.Cg-Tg(SOD1^{G93A})1Gur/J mice (Jackson Laboratory, Bar Harbor, ME) was used after in vitro fertilization (Macrogen, Seoul, Korea) and all the protocol was approved by the Institutional Animal Care and Use Committees of Dong-A University.

### Mouse Survival and Efficacy Evaluation

Caffeic acid (30) mg/kg) dissolved in PBS containing 10% ethanol or vehicle (PBS containing 10% ethanol) were orally administered 5 days/week from 60 days to 120 days of age. Three independent experiments were performed to evaluate the efficacy of caffeic acid. A total of 24 mice in each group (female=12, male=12) were used for evaluation of survival and behavioral assessments (Exp 1), and the same number of mice were used for histologic analyses (Exps 2 and 3). In the Exp 1, mice were monitored for neurological disease progression according to guidelines for preclinical animal research in ALS/MND (Ludolph AC et al, 2010). The neurological score was followed as Score 0.5 as disease onset (first signs of tremor and hind-limb splay defects) and the end stage (Score 4) was determined as loss of righting reflex within 30s. Neurological scoring was monitored daily and mice at the end stage were euthanized. Kaplan-Meier curves was used to analyze age of onset and survival using Graphpad Prism7 (GraphPad Software, San Diego, CA). Motor coordination and muscle integrity were assessed weekly using a Rotarod apparatus and grip strength device (Panlab Harvard Apparatus, Barcelona, Spain). Tissue analyses were performed in the Exp 2 (n=14 for each group) and Exp 3 (n=10 for each group). They were anesthetized with euthanized isofluran at 16 wks (Exp 2) and 20 wks (Exp 3) of age then perfused with 4% paraformaldehyde in PBS. L4-L5 segments of spinal cord were serially cut with the cryostat into 20 μm sections then stained with 0.1% (w/v) cresyl violet stain solution. Motor neurons with larger than 25 μm of dimeter in Lamina IX of the ventral horn were counted using Image J software (National Institutes of Health, Bethesda, USA) program, and 10) sections per sample (n=10 for each group) were averaged. Activated astrocytes and microglia in the spinal cord were detected using anti-GFAP Ab and rabbit anti-Iba1 Ab (Cell Signaling Technology, Beverly, MA) for 10 sections per sample (n=10 for each group). The integrated density of fraction area in the ventral horn were measured using Image J software for quantification of activated astrocytes and microglia. Neuromuscular junction was analyzed in gastrocnemius muscle (30 μm) with anti-a-bungarotoxin Ab to label AChR and anti-neurofilament H/synapsin Ab (Cell Signaling Technology) to label axon terminals. The innervated pretzel structures merged with two fluorescence were counted.

### Whole Mount Staining

For whole mount immunostaining, E11.5 embryos were fixed in 4% PFA, permeabilized in PBS-T (1% Triton X-100 in PBS), blocked using blocking buffer (1% heat inactivated goat serum, 1% Triton X-100 in PBS) at 4° C. Embryos were incubated 3-5 days at 4° C. with rabbit anti-GFP (Invitrogen) primary antibody in blocking buffer. Fluorophore-conjugated secondary antibody (Invitrogen) was incubated for 1 day at 4° C., and images were captured using a Zeiss confocal microscope¹⁰⁹.

### Fly Stocks and Culture

Flies were maintained on a cornmeal-molasses-yeast medium at room temperature (22° C.) with 60-65% humidity. The following Drosophila lines were obtained from the Bloomington Stock Center: elav-GAL4, GMR-GAL4, and OK371-GAL4. The UAS-(G₄C₂)₃ and UAS-(G₄C₂)₃₀ lines were obtained from Dr. Peng Jin's laboratory⁸¹.

### Fly Eye Degeneration Experiment

UAS-(G₄C₂)₃₀ flies recombined with GMR-Gal4 were selected as male parental flies for crossing (♀w¹¹¹⁸×♂GMR-Gal4; UAS-(G₄C₂)₃₀/CyO). Overexpressing 30 hexanucleotide repeat (HRE) in all photoreceptors using GMR-Gal4 causes eye degeneration in adult flies during aging. Eye degeneration scores were examined based on Dr. Paul Taylor's study¹¹⁰. Data of eye degeneration was quantified for the presence of: supernumerary inter-ommatidial bristles (IOBs), IOBs with abnormal orientation, necrotic patches, a decrease in size, retinal collapse, fusion of ommatidia, disorganization of ommatidial array and loss of pigmentation in adult male progeny. Points were added if: there was complete loss of IOBs (+1), more than 3 small or 1 large necrotic patch (+1), retinal collapse extended to the midline of the eye (+1) or beyond (+2), loss of ommatidial structure in less than 50% (+1) or more than 50% (+2) of the eye, and if pigmentation loss resulted in change of eye color from red to orange (+1) or pale orange/white (+2).

### Fly Survival Experiment

UAS-(G₄C₂)₃₀ flies recombined with OK371-Gal4 were selected as male parental flies for crossing (♀w¹¹¹⁸×♂OK371-Gal4: UAS-(G₄C₂)₃₀/TM6B, GAL80). Overexpressing 30 HRE in fly motor neurons using OK371-Gal4 causes lethality due to paralysis, preventing eclosion of the adult from the pupal case. According to Mendelian inheritance, the theoretical ratio of progenies with 30 HRE expressions from the above crossing is 50%. A total of 100 adult flies were collected in each group. Survival rate was calculated as the ratio of the flies with 30 HRE that survive to adulthood to total adult flies and then divided by theoretical ratio 50%.

### Statistical Analysis

Each value is from at least 3 different experiments of multiple batches and reported as mean±SEM. Statistical differences between samples were analyzed by unpaired Student's t-test in GraphPad Prism 7 and indicated the p-value level in each legend.

### REFERENCES

1 Tandan, R. & Bradley, W. G. Amyotrophic lateral sclerosis: Part 1. Clinical features, pathology, and ethical issues in management. Annals of Neurology: Official Journal of the American Neurological Association and the Child Neurology Society 18, 271-280 (1985).
2 Gurney, M. E. et al. Motor neuron degeneration in mice that express a human Cu, Zn superoxide dismutase mutation. Science 264, 1772-1775 (1994).
3 Taylor, J. P., Brown Jr, R. H. & Cleveland, D. W. Decoding ALS: from genes to mechanism. Nature 539, 197 (2016).
4 Saxena, S. & Caroni, P. Selective neuronal vulnerability in neurodegenerative diseases: from stressor thresholds to degeneration. Neuron 71, 35-48 (2011).
5 DeJesus-Hernandez, M. et al. Expanded GGGGCC hexanucleotide repeat in noncoding region of C9ORF72 causes chromosome 9p-linked FTD and ALS. Neuron 72, 245-256 (2011).
6 Mackenzie, I. R. & Rademakers, R. The role of TDP-43 in amyotrophic lateral sclerosis and frontotemporal dementia. Current opinion in neurology 21, 693 (2008).
7 Sharma, A. et al. ALS-associated mutant FUS induces selective motor neuron degeneration through toxic gain of function. Nature communications 7, 10465 (2016).
8 Maruyama, H. et al. Mutations of optineurin in amyotrophic lateral sclerosis. Nature 465, 223 (2010).
9 Wu, C.-H. et al. Mutations in the profilin 1 gene cause familial amyotrophic lateral sclerosis. Nature 488, 499 (2012).
10 Johnson, J. O. et al. Mutations in the Matrin 3 gene cause familial amyotrophic lateral sclerosis. Nature neuroscience 17, 664 (2014).
11 Smith, B. N. et al. Exome-wide rare variant analysis identifies TUBA4A mutations associated with familial ALS. Neuron 84, 324-331 (2014).
12 Freischmidt, A. et al. Haploinsufficiency of TBK1 causes familial ALS and fronto-temporal dementia. Nature neuroscience 18, 631 (2015).
13 Lattante, S., Ciura, S., Rouleau, G. A. & Kabashi, E. Defining the genetic connection linking amyotrophic lateral sclerosis (ALS) with frontotemporal dementia (FTD). Trends in Genetics 31, 263-273 (2015).
14 Philips, T. & Rothstein, J. D. Rodent models of amyotrophic lateral sclerosis. Current protocols in pharmacology 69, 5.67. 61-65.67. 21 (2015).
15 Van Damme, P., Robberecht, W. & Van Den Bosch, L. Modelling amyotrophic lateral sclerosis: progress and possibilities. Disease models & mechanisms 10, 537-549 (2017).
16 Fujimori, K. et al. Modeling sporadic ALS in iPSC-derived motor neurons identifies a potential therapeutic agent. Nature medicine 24, 1579 (2018).
17 Soldner, F. et al. Parkinson-associated risk variant in distal enhancer of a-synuclein modulates target gene expression. Nature 533, 95 (2016).
18 Lee, G. et al. Modelling pathogenesis and treatment of familial dysautonomia using patient-specific iPSCs. Nature 461, 402 (2009).
19 Kondo, T. et al. Modeling Alzheimer's disease with iPSCs reveals stress phenotypes associated with intracellular AB and differential drug responsiveness. Cell stem cell 12, 487-496 (2013).
20 Wainger, B. J. et al. Modeling pain in vitro using nociceptor neurons reprogrammed from fibroblasts. Nature neuroscience 18, 17 (2015).
21 Kikuchi, T. et al. Human iPS cell-derived dopaminergic neurons function in a primate Parkinson's disease model. Nature 548, 592 (2017).
22 Lopez-Gonzalez, R. et al. Poly (GR) in C9ORF72-related ALS/FTD compromises mitochondrial function and increases oxidative stress and DNA damage in iPSC-derived motor neurons. Neuron 92, 383-391 (2016).
23 Klim, J. R. et al. ALS-implicated protein TDP-43 sustains levels of STMN2, a mediator of motor neuron growth and repair. Nature neuroscience 22, 167 (2019).
24 Melamed, Z. e. et al. Premature polyadenylation-mediated loss of stathmin-2 is a hallmark of TDP-43-dependent neurodegeneration. Nature neuroscience 22, 180 (2019).
25 Kiskinis, E. et al. Pathways disrupted in human ALS motor neurons identified through genetic correction of mutant SOD1. Cell stem cell 14, 781-795 (2014).
26 Marchetto, M. C. et al. Non-cell-autonomous effect of human SOD1G37R astrocytes on motor neurons derived from human embryonic stem cells. Cell stem cell 3, 649-657 (2008).
27 Ling, S.-C. et al. ALS-associated mutations in TDP-43 increase its stability and promote TDP-43 complexes with FUS/TLS. Proceedings of the National Academy of Sciences 107, 13318-13323 (2010).
28 Soldner, F. et al. Generation of isogenic pluripotent stem cells differing exclusively at two early onset Parkinson point mutations. Cell 146, 318-331 (2011).
29 Cho, S. W. et al. Analysis of off-target effects of CRISPR/Cas-derived RNA-guided endonucleases and nickases. Genome research 24, 132-141 (2014).
30 Fu, Y. et al. High-frequency off-target mutagenesis induced by CRISPR-Cas nucleases in human cells. Nature biotechnology 31, 822 (2013).
31 Gizzi, M., DiRocco, A., Sivak, M. & Cohen, B. Ocular motor function in motor neuron disease. Neurology 42, 1037-1037 (1992).
32 Kaminski, H. J., Richmonds, C. R., Kusner, L. L. & Mitsumoto, H. Differential susceptibility of the ocular motor system to disease. Annals of the New York Academy of Sciences 956, 42-54 (2002).
33 Kanning, K. C., Kaplan, A. & Henderson, C. E. Motor neuron diversity in development and disease. Annual review of neuroscience 33, 409-440 (2010).
34 Cleveland, D. W. & Rothstein, J. D. From Charcot to Lou Gehrig: deciphering selective motor neuron death in ALS. Nature reviews neuroscience 2, 806 (2001).
35 Kaplan, A. et al. Neuronal matrix metalloproteinase-9 is a determinant of selective neurodegeneration. Neuron 81, 333-348 (2014).
36 Allodi, I. et al. Differential neuronal vulnerability identifies IGF-2 as a protective factor in ALS. Scientific reports 6, 25960 (2016).
37 Mazzoni, E. O. et al. Synergistic binding of transcription factors to cell-specific enhancers programs motor neuron identity. Nature neuroscience 16, 1219 (2013).
38 Allodi, I. et al. Modeling motor neuron resilience in ALS using stem cells. Stem cell reports (2019).
39 Tafuri, F., Ronchi, D., Magri, F., Comi, G. P. & Corti, S. SOD1 misplacing and mitochondrial dysfunction in amyotrophic lateral sclerosis pathogenesis. Frontiers in cellular neuroscience 9, 336 (2015).
40 Lewerenz, J. & Maher, P. Chronic glutamate toxicity in neurodegenerative diseases-what is the evidence? Frontiers in neuroscience 9, 469 (2015).
41 Blizzard, C. A., Lee, K. & Dickson, T. C. Inducing chronic excitotoxicity in the mouse spinal cord to investigate lower motor neuron degeneration. Frontiers in neuroscience 10, 76 (2016).
42 Maragakis, N. J. & Rothstein, J. D. Mechanisms of disease: astrocytes in neurodegenerative disease. Nature Reviews Neurology 2, 679 (2006).
43 Haidet-Phillips, A. M. et al. Astrocytes from familial and sporadic ALS patients are toxic to motor neurons. Nature biotechnology 29, 824 (2011).
44 Cutler, R. G., Pedersen, W. A., Camandola, S., Rothstein, J. D. & Mattson, M. P. Evidence that accumulation of ceramides and cholesterol esters mediates oxidative stress-induced death of motor neurons in amyotrophic lateral sclerosis. Annals of Neurology: Official Journal of the American Neurological Association and the Child Neurology Society 52, 448-457 (2002).
45 Theofilopoulos, S. et al. Cholestenoic acids regulate motor neuron survival via liver X receptors. The Journal of clinical investigation 124, 4829-4842 (2014).
46 Abdel-Khalik, J. et al. Defective cholesterol metabolism in amyotrophic lateral sclerosis. Journal of lipid research 58, 267-278 (2017).
47 Pattyn, A., Hirsch, M., Goridis, C. & Brunet, J.-F. Control of hindbrain motor neuron differentiation by the homeobox gene Phox2b. Development 127, 1349-1358 (2000).
48 Prakash, N. et al. Nkx6-1 controls the identity and fate of red nucleus and oculomotor neurons in the mouse midbrain. Development 136, 2545-2555 (2009).
49 Hasan, K. B., Agarwala, S. & Ragsdale, C. W. PHOX2A regulation of oculomotor complex nucleogenesis. Development 137, 1205-1213 (2010).
50 Deng, Q. et al. Specific and integrated roles of Lmxla, Lmx1b and Phox2a in ventral midbrain development. Development 138, 3399-3408 (2011).
51 Mong, J. et al. Transcription Factor-Induced Lineage Programming of Noradrenaline and Motor Neurons from Embryonic Stem Cells. Stem Cells 32, 609-622 (2014).
52 Song, M.-R. et al. T-Box transcription factor Tbx20 regulates a genetic program for cranial motor neuron cell body migration. Development 133, 4945-4955 (2006).
53 Nakano, M. et al. Homozygous mutations in ARIX (PHOX2A) result in congenital fibrosis of the extraocular muscles type 2. Nature genetics 29, 315 (2001).
54 Amiel, J. et al. Polyalanine expansion and frameshift mutations of the paired-like homeobox gene PHOX2B in congenital central hypoventilation syndrome. Nature genetics 33, 459 (2003).
55 Raabe, E. et al. Prevalence and functional consequence of PHOX2B mutations in neuroblastoma. Oncogene 27, 469 (2008).
56 Oh, Y. et al. Functional coupling with cardiac muscle promotes maturation of hPSC-derived sympathetic neurons. Cell Stem Cell 19, 95-106 (2016).
57 Kriks, S. et al. Dopamine neurons derived from human ES cells efficiently engraft in animal models of Parkinson's disease. Nature 480, 547 (2011).
58 Tang, M., Luo, S. X., Tang, V. & Huang, E. J. Temporal and spatial requirements of Smoothened in ventral midbrain neuronal development. Neural development 8, 8 (2013).
59 Danielian, P. S. & McMahon, A. P. Engrailed-1 as a target of the Wnt-1 signalling pathway in vertebrate midbrain development. Nature 383, 332 (1996).
60 Tsarovina, K. et al. Essential role of Gata transcription factors in sympathetic neuron development. Development 131, 4775-4786 (2004).
61 Arber, S. et al. Requirement for the homeobox gene Hb9 in the consolidation of motor neuron identity. Neuron 23, 659-674 (1999).
62 Thaler, J. et al. Active suppression of interneuron programs within developing motor neurons revealed by analysis of homeodomain factor HB9. Neuron 23, 675-687 (1999).
63 Song, M.-R. et al. Islet-to-LMO stoichiometries control the function of transcription complexes that specify motor neuron and V2a interneuron identity. Development 136, 2923-2932 (2009).
64 Lewcock, J. W., Genoud, N., Lettieri, K. & Pfaff, S. L. The ubiquitin ligase Phrl regulates axon outgrowth through modulation of microtubule dynamics. Neuron 56, 604-620 (2007).
65 Guidato, S., Barrett, C. & Guthrie, S. Patterning of motor neurons by retinoic acid in the chick embryo hindbrain in vitro. Molecular and Cellular Neuroscience 23, 81-95 (2003).
66 Lance-Jones, C., Shah, V., Noden, D. M. & Sours, E. Intrinsic properties guide proximal abducens and oculomotor nerve outgrowth in avian embryos. Developmental neurobiology 72, 167-185 (2012).
67 Calder, E. L. et al. Retinoic acid-mediated regulation of GLI3 enables efficient motoneuron derivation from human ESCs in the absence of extrinsic SHH activation. Journal of Neuroscience 35, 11462-11481 (2015).
68 Allodi, I. et al. Modeling motor neuron resilience in ALS using stem cells. Stem cell reports 12, 1329-1341 (2019).
69 Hedlund, E., Karlsson, M., Osborn, T., Ludwig, W. & Isacson, O. Global gene expression profiling of somatic motor neuron populations with different vulnerability identify molecules and pathways of degeneration and protection. Brain 133, 2313-2330 (2010).
70 Valbuena, G. N. et al. Metabolomic analysis reveals increased aerobic glycolysis and amino acid deficit in a cellular model of amyotrophic lateral sclerosis. Molecular neurobiology 53, 2222-2240 (2016).
71 Xia, J. & Wishart, D. S. Using MetaboAnalyst 3.0 for comprehensive metabolomics data analysis. Current protocols in bioinformatics 55, 14.10. 11-14.10. 91 (2016).
72 Lawton, K. A. et al. Plasma metabolomic biomarker panel to distinguish patients with amyotrophic lateral sclerosis from disease mimics. Amyotrophic Lateral Sclerosis and Frontotemporal Degeneration 15, 362-370 (2014).
73 Jatana, M. et al. Inhibition of NF-κB activation by 5-lipoxygenase inhibitors protects brain against injury in a rat model of focal cerebral ischemia. Journal of neuroinflammation 3, 12 (2006).
74 Häfner, A.-K., Kahnt, A. S. & Steinhilber, D. Beyond leukotriene formation—the noncanonical functions of 5-lipoxygenase. Prostaglandins & other lipid mediators (2019).
75 Cao, Y., Pearman, A. T., Zimmerman, G. A., McIntyre, T. M. & Prescott, S. M. Intracellular unesterified arachidonic acid signals apoptosis. Proceedings of the National Academy of Sciences 97, 11280-11285 (2000).
76 Das, U. N. Arachidonic acid in health and disease with focus on hypertension and diabetes mellitus: A review. Journal of advanced research 11, 43-55 (2018).
77 Rizzo, M. T. et al. Induction of apoptosis by arachidonic acid in chronic myeloid leukemia cells. Cancer research 59, 5047-5053 (1999).
78 Yang, J. Q., Zhou, Q. X., Liu, B. Z. & He, B. C. Protection of Mouse Brain from Aluminum-induced Damage by Caffeic Acid. CNS neuroscience & therapeutics 14, 10-16 (2008).
79 Bishayee, K. & Khuda-Bukhsh, A. R. 5-lipoxygenase antagonist therapy: a new approach towards targeted cancer chemotherapy. Acta Biochim Biophys Sin 45, 709-719 (2013).
80 Pergola, C. & Werz, O. 5-Lipoxygenase inhibitors: a review of recent developments and patents. Expert opinion on therapeutic patents 20, 355-375 (2010).
81 Xu, Z. et al. Expanded GGGGCC repeat RNA associated with amyotrophic lateral sclerosis and frontotemporal dementia causes neurodegeneration. Proceedings of the National Academy of Sciences 110, 7778-7783 (2013).
82 An, D. et al. Stem cell-derived cranial and spinal motor neurons reveal proteostatic differences between ALS resistant and sensitive motor neurons. eLife 8, e44423 (2019).
83 Kuehne, A. et al. Acute activation of oxidative pentose phosphate pathway as first-line response to oxidative stress in human skin cells. Molecular cell 59, 359-371 (2015).
84 Patra, K. C. & Hay, N. The pentose phosphate pathway and cancer. Trends in biochemical sciences 39, 347-354 (2014).
85 Andrus, P. K., Fleck, T. J., Gurney, M. E. & Hall, E. D. Protein oxidative damage in a transgenic mouse model of familial amyotrophic lateral sclerosis. Journal of neurochemistry 71, 2041-2048 (1998).
86 Dodge, J. C. et al. Glycosphingolipids are modulators of disease pathogenesis in amyotrophic lateral sclerosis. Proceedings of the National Academy of Sciences 112, 8100-8105 (2015).
87 Tallima, H. & El Ridi, R. Arachidonic acid: physiological roles and potential health benefits-a review. Journal of advanced research 11, 33-41 (2018).
88 West, M. et al. The arachidonic acid 5-lipoxygenase inhibitor nordihydroguaiaretic acid inhibits tumor necrosis factor a activation of microglia and extends survival of G93A-SOD1 transgenic mice. Journal of neurochemistry 91, 133-143 (2004).
89 Ran, F. A. et al. Genome engineering using the CRISPR-Cas9 system. Nature protocols 8, 2281 (2013).
90 Hockemeyer, D. et al. Genetic engineering of human pluripotent cells using TALE nucleases. Nature biotechnology 29, 731 (2011).
91 Mali, P. et al. RNA-guided human genome engineering via Cas9. Science 339, 823-826 (2013).
92 Gendron, T. F. et al. Poly (GP) proteins are a useful pharmacodynamic marker for C9ORF72-associated amyotrophic lateral sclerosis. Science translational medicine 9, eaai7866 (2017).
93 Almad, A. A. et al. Connexin 43 in astrocytes contributes to motor neuron toxicity in amyotrophic lateral sclerosis. Glia 64, 1154-1169 (2016).
94 Choi, I. Y. et al. Concordant but varied phenotypes among Duchenne muscular dystrophy patient-specific myoblasts derived using a human iPSC-based model. Cell reports 15, 2301-2312 (2016).
95 Chambers, S. M. et al. Highly efficient neural conversion of human ES and iPS cells by dual inhibition of SMAD signaling. Nature biotechnology 27, 275 (2009).
96 Qi, Y. et al. Combined small-molecule inhibition accelerates the derivation of functional cortical neurons from human pluripotent stem cells. Nature biotechnology 35, 154 (2017). 97 Lee, H. et al. Slit and Semaphorin signaling governed by Islet transcription factors positions motor neuron somata within the neural tube. Experimental neurology 269, 17-27 (2015).
98 Pattyn, A., Morin, X., Cremer, H., Goridis, C. & Brunet, J.-F. Expression and interactions of the two closely related homeobox genes Phox2a and Phox2b during neurogenesis. Development 124, 4065-4075 (1997).
99 Ng, S.-Y. et al. Genome-wide RNA-Seq of human motor neurons implicates selective ER stress activation in spinal muscular atrophy. Cell stem cell 17, 569-584 (2015).
100 Dobin, A. et al. STAR: ultrafast universal RNA-seq aligner. Bioinformatics 29, 15-21 (2013).
101 Robinson, M. D., McCarthy, D. J. & Smyth, G. K. edgeR: a Bioconductor package for differential expression analysis of digital gene expression data. Bioinformatics 26, 139-140 (2010).
102 Law, C. W., Chen, Y., Shi, W. & Smyth, G. K. voom: Precision weights unlock linear model analysis tools for RNA-seq read counts. Genome biology 15, R29 (2014).
103 McCarthy, D. J. & Smyth, G. K. Testing significance relative to a fold-change threshold is a TREAT. Bioinformatics 25, 765-771 (2009).
104 Subramanian, A. et al. Gene set enrichment analysis: a knowledge-based approach for interpreting genome-wide expression profiles. Proceedings of the National Academy of Sciences 102, 15545-15550 (2005).
105 Yu, G., Wang, L.-G., Han, Y. & He, Q.-Y. clusterProfiler: an R package for comparing biological themes among gene clusters. Omics: a journal of integrative biology 16, 284-287 (2012).
106 Eoh, H. & Rhee, K. Y. Multifunctional essentiality of succinate metabolism in adaptation to hypoxia in Mycobacterium tuberculosis. Proceedings of the National Academy of Sciences 110, 6554-6559 (2013).
107 Lee, S.-K., Jurata, L. W., Funahashi, J., Ruiz, E. C. & Pfaff, S. L. Analysis of embryonic motoneuron gene regulation: derepression of general activators function in concert with enhancer factors. Development 131, 3295-3306 (2004).
108 Bai, G. et al. Presenilin-dependent receptor processing is required for axon guidance. Cell 144, 106-118 (2011).
109 Kim, K.-T. et al. ISL1-based LIM complexes control Slit2 transcription in developing cranial motor neurons. Scientific reports 6, 36491 (2016).
110 Ritson, G. P. et al. TDP-43 mediates degeneration in a novel Drosophila model of disease caused by mutations in VCP/p97. Journal of Neuroscience 30, 7729-7739 (2010).
   Allodi I, Nijssen J, Benitez J A, Schweingruber C, Fuchs A, Bonvicini G, Cao M, Kiehn O, Hedlund E. Modeling Motor Neuron Resilience in ALS Using Stem Cells. Stem Cell Reports. 2019 Jun. 11; 12(6):1329-1341.
   Blasco H, Veyrat-Durebex C, Bocca C, Patin F, Vourc'h P, Kouassi Nzoughet J, Lenaers G, Andres C R, Simard G, Corcia P, Reynier P. Lipidomics Reveals Cerebrospinal-Fluid Signatures of ALS. Sci Rep. 2017 Dec. 15; 7(1):17652.
   Chaves-Filho A B, Pinto I F D, Dantas L S, Xavier A M, Inague A, Faria R L, Medeiros M H G, Glezer I, Yoshinaga M Y, Miyamoto S. Alterations in lipid metabolism of spinal cord linked to amyotrophic lateral sclerosis. Sci Rep. 2019 Aug. 12; 9(1):11642.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

1. A method of treating an amyotrophic lateral sclerosis (ALS) cell, comprising:
selecting a therapeutic compound;
treating an aberrant arachidonic acid (AA) metabolic pathway in said ALS cell comprising contacting said ALS cell with said therapeutic compound.

2. The method of claim 1, wherein said treating said aberrant arachidonic acid (AA) metabolic pathway results in a reduction of a cellular level of AA in said ALS cell.

3. The method of claim 2, wherein said therapeutic compound is an inhibitor of 5-lipoxygenase (5-LOX).

4. The method of claim 3, wherein said inhibitor of 5-LOX comprises a redox-active inhibitor.

5. The method of claim 3, wherein said inhibitor of 5-LOX comprises caffeic acid (3,4-dihydroxybenenearcrylic acid), apigenin, BW755C, nordihydroguaretic acid, or a functional analog or derivative thereof.

6. A method of treating a subject with ALS, comprising:
selecting a therapeutic compound;
treating an aberrant arachidonic acid (AA) metabolic pathway in said subject comprising administering to said subject said therapeutic compound.

7. The method of claim 6, wherein said therapeutic compound results in a reduction of a cellular level of AA in said spinal motor neuron cell of said subject.

8. The method of claim 7, wherein said therapeutic compound is an inhibitor of 5-lipoxygenase (5-LOX).

9. The method of claim 8, wherein said inhibitor of 5-LOX comprises a redox-active compound, an iron ligand inhibitor, a non-redox-type inhibitor, a redox-type inhibitor, a Dual (COX/5-LOX) type inhibitor, or an iron ligand-type inhibitor.

10. The method of claim 9, wherein said inhibitor of 5-LOX comprises a redox-active inhibitor.

11. A method of differentiating a human stem cell to an ocular motor neuron (oMN) ALS-specific human cell type, comprising:
culturing said human stem cell in a first media comprising recombinant sonic hedgehog signaling protein and purmorphamine for 9 days;
culturing said human stem cell in a second media comprising brain-derived neurotrophic factor (BDNF), glial cell line-derived neurotrophic factor (GDNF), and Ascorbic Acid for at least 1 day,
wherein said second media does not comprise sonic hedgehog signaling protein or purmorphamine.

12. The method of claim 11, wherein said human stem cell is an embryonic human stem cell or a human induced pluripotent stem cell.

13. The method of claim 12, wherein an expression of at least one oMN-specific gene is increased in said oMN ALS-specific human cell.

14. The method of claim 13, wherein said at least one oMN-specific gene is selected from the list consisting of ISL1, PHOX2A, NKX6.1, EN1, CHAT, PHOX2B, TBX20, FGF10, EYA1, EYA2, PLEXINA4, SEMA6D and MAP2.

15. An ocular motor neuron (oMN) ALS-specific human cell generated from the method of claim 11.

16. A method for identifying whether a metabolic pathway is dysregulated in a sMN ALS cell, comprising:
isolating said sMN ALS cell;
isolating an oMN ALS cell;
isolating total RNA from said sMN cell;
isolating total RNA from said oMN cell; and
performing a differential gene expression assay from said total RNA from said sMN cell and from said total RNA from said oMN cell, said differential gene expression assay comprising comparing an expression level of a gene associated with said metabolic pathway from said sMN ALS cell with an expression level of said gene associated with said metabolic pathway from said OMN ALS cell;
wherein a difference in the expression level of said gene associated with said metabolic pathway from said sMN ALS cell as compared to the expression level of said gene associated with said metabolic pathway from said oMN ALS cell is indicative of a dysregulation of said metabolic pathway.

17. The method of claim 16, wherein said sMN ALS cell is differentiated from a human stem cell.

18. The method of claim 16, wherein said oMN ALS cell is differentiated from a human stem cell.

19. The method of claim 16, further comprising determining whether a metabolite associated with said metabolic pathway is dysregulated in said sMN ALS cell, comprising:
isolating said metabolite from said sMN cell;
isolating said metabolite from said oMN cell;
determining the relative abundance of said metabolite from said sMN cell;
determining the relative abundance of said metabolite from said oMN cell; and
comparing the relative abundance of said metabolite from said sMN cell with the relative abundance of said metabolite from said oMN cell,
wherein a difference in the relative abundance of said metabolite from said sMN cell as compared to the relative abundance of said metabolite from said oMN cell indicative of a dysregulation of said metabolic pathway.

20. The method of claim 3, wherein said inhibitor of 5-LOX comprises a redox-active compound, an iron ligand inhibitor, a non-redox-type inhibitor, a redox-type inhibitor, a Dual (COX/5-LOX) type inhibitor, or an iron ligand-type inhibitor.


SEQUENCE LISTING (88 sequences)

SEQ ID NO: 1 - DNA, 34 bp, Artificial Sequence
  Description of Artificial Sequence Synthetic oligonucleotide
ataggatcctcaactcctgggcttcccggaacct

SEQ ID NO: 2 - DNA, 34 bp, Artificial Sequence
  Description of Artificial Sequence Synthetic oligonucleotide
atagctagcctggggcgcgggctggtggctgggc

SEQ ID NO: 3 - DNA, 36 bp, Artificial Sequence
  Description of Artificial Sequence Synthetic oligonucleotide
ataggcgcgccgagccccgcgcccagcaggtgcggc

SEQ ID NO: 4 - DNA, 36 bp, Artificial Sequence
  Description of Artificial Sequence Synthetic oligonucleotide
atagcggccgccccgggacaggtgtgcaccaggcag

SEQ ID NO: 5 - DNA, 25 bp, Artificial Sequence
  Description of Artificial Sequence Synthetic oligonucleotide
caccgtacagcaacggcgccagcgt

SEQ ID NO: 6 - DNA, 25 bp, Artificial Sequence
  Description of Artificial Sequence Synthetic oligonucleotide
aaacacgctggcgccgttgctgtac

SEQ ID NO: 7 - DNA, 25 bp, Artificial Sequence
  Description of Artificial Sequence Synthetic oligonucleotide
caccgcggaggacgactcgccgccc

SEQ ID NO: 8 - DNA, 25 bp, Artificial Sequence
  Description of Artificial Sequence Synthetic oligonucleotide
aaacgggcggcgagtcgtcctccgc

SEQ ID NO: 9 - DNA, 20 bp, Artificial Sequence
  Description of Artificial Sequence Synthetic primer
tgcaccaccaactgcttagc

SEQ ID NO: 10 - DNA, 21 bp, Artificial Sequence
  Description of Artificial Sequence Synthetic primer
ggcatggactgtggtcatgag

SEQ ID NO: 11 - DNA, 20 bp, Artificial Sequence
  Description of Artificial Sequence Synthetic primer
agtgagaggcaacctggaga

SEQ ID NO: 12 - DNA, 20 bp, Artificial Sequence
  Description of Artificial Sequence Synthetic primer
acactcggaccacatccttc

SEQ ID NO: 13 - DNA, 20 bp, Artificial Sequence
  Description of Artificial Sequence Synthetic primer
catgagtgtggatccagctt

SEQ ID NO: 14 - DNA, 20 bp, Artificial Sequence
  Description of Artificial Sequence Synthetic primer
cctgaataagcagatccatg

SEQ ID NO: 15 - DNA, 20 bp, Artificial Sequence
  Description of Artificial Sequence Synthetic primer
ggtttctccggatttggaat

SEQ ID NO: 16 - DNA, 20 bp, Artificial Sequence
  Description of Artificial Sequence Synthetic primer
cacgaagtcgttcttgctga

SEQ ID NO: 17 - DNA, 20 bp, Artificial Sequence
  Description of Artificial Sequence Synthetic primer
tctcgctgtctctccctctc

SEQ ID NO: 18 - DNA, 20 bp, Artificial Sequence
  Description of Artificial Sequence Synthetic primer
cgtggcttactccccattta

SEQ ID NO: 19 - DNA, 22 bp, Artificial Sequence
  Description of Artificial Sequence Synthetic primer
ccgatggactactcctacctca

SEQ ID NO: 20 - DNA, 20 bp, Artificial Sequence
  Description of Artificial Sequence Synthetic primer
gcagggggctgtattggaag

SEQ ID NO: 21 - DNA, 20 bp, Artificial Sequence
  Description of Artificial Sequence Synthetic primer
cggtcctgcacagattccca

SEQ ID NO: 22 - DNA, 20 bp, Artificial Sequence
  Description of Artificial Sequence Synthetic primer
cagcagcttcggcctcaaag

SEQ ID NO: 23 - DNA, 20 bp, Artificial Sequence
  Description of Artificial Sequence Synthetic primer
cggaggaggtggatgtgctt

SEQ ID NO: 24 - DNA, 20 bp, Artificial Sequence
  Description of Artificial Sequence Synthetic primer
gccctgaccgagtttccgta

SEQ ID NO: 25 - DNA, 22 bp, Artificial Sequence
  Description of Artificial Sequence Synthetic primer
caagtcacatgggcagagatag

SEQ ID NO: 26 - DNA, 22 bp, Artificial Sequence
  Description of Artificial Sequence Synthetic primer
ggctaggagggttacagaaatg

SEQ ID NO: 27 - DNA, 20 bp, Artificial Sequence
  Description of Artificial Sequence Synthetic primer
acaagtacctttgcagaccc

SEQ ID NO: 28 - DNA, 21 bp, Artificial Sequence
  Description of Artificial Sequence Synthetic primer
catttcccttcgctgttttgg

SEQ ID NO: 29 - DNA, 20 bp, Artificial Sequence
  Description of Artificial Sequence Synthetic primer
tttagccgttcgcttagagg

SEQ ID NO: 30 - DNA, 20 bp, Artificial Sequence
  Description of Artificial Sequence Synthetic primer
cggatagctggagacaggag

SEQ ID NO: 31 - DNA, 20 bp, Artificial Sequence
  Description of Artificial Sequence Synthetic primer
ggccttccgtccctgagtat

SEQ ID NO: 32 - DNA, 20 bp, Artificial Sequence
  Description of Artificial Sequence Synthetic primer
gcaacgagaacagggcttct

SEQ ID NO: 33 - DNA, 20 bp, Artificial Sequence
  Description of Artificial Sequence Synthetic primer
tctccctcctctgtcaccct

SEQ ID NO: 34 - DNA, 20 bp, Artificial Sequence
  Description of Artificial Sequence Synthetic primer
cccgaccctctgtcctcatt

SEQ ID NO: 35 - DNA, 20 bp, Artificial Sequence
  Description of Artificial Sequence Synthetic primer
gcctgaggtcaatggtgcaa

SEQ ID NO: 36 - DNA, 20 bp, Artificial Sequence
  Description of Artificial Sequence Synthetic primer
aaagtcaagcccgtttgcca

SEQ ID NO: 37 - DNA, 19 bp, Artificial Sequence
  Description of Artificial Sequence Synthetic primer
gcaccagttcaagctcaac

SEQ ID NO: 38 - DNA, 20 bp, Artificial Sequence
  Description of Artificial Sequence Synthetic primer
gctgcgtttccatttcatcc

SEQ ID NO: 39 - DNA, 23 bp, Artificial Sequence
  Description of Artificial Sequence Synthetic primer
agacaaaaagtaacggttcagcc

SEQ ID NO: 40 - DNA, 21 bp, Artificial Sequence
  Description of Artificial Sequence Synthetic primer
cgcactctagtaagtggttgc

SEQ ID NO: 41 - DNA, 20 bp, Artificial Sequence
  Description of Artificial Sequence Synthetic primer
gctgggcccgggaaagttcg

SEQ ID NO: 42 - DNA, 21 bp, Artificial Sequence
  Description of Artificial Sequence Synthetic primer
gtgctagcagcaggtcgcctc

SEQ ID NO: 43 - DNA, 20 bp, Artificial Sequence
  Description of Artificial Sequence Synthetic primer
gagatgaagaccccgctgta

SEQ ID NO: 44 - DNA, 18 bp, Artificial Sequence
  Description of Artificial Sequence Synthetic primer
gacgacgacgaggacgag

SEQ ID NO: 45 - DNA, 23 bp, Artificial Sequence
  Description of Artificial Sequence Synthetic primer
caggagacagagatgagaattcc

SEQ ID NO: 46 - DNA, 20 bp, Artificial Sequence
  Description of Artificial Sequence Synthetic primer
caggagtgatggcagtagac

SEQ ID NO: 47 - DNA, 18 bp, Artificial Sequence
  Description of Artificial Sequence Synthetic primer
gacgtctgacgggaggag

SEQ ID NO: 48 - DNA, 20 bp, Artificial Sequence
  Description of Artificial Sequence Synthetic primer
tcaatcatgtccagcgagtc

SEQ ID NO: 49 - DNA, 20 bp, Artificial Sequence
  Description of Artificial Sequence Synthetic primer
aggagggcaagagaggtcca

SEQ ID NO: 50 - DNA, 20 bp, Artificial Sequence
  Description of Artificial Sequence Synthetic primer
accagccaccactcaggaac

SEQ ID NO: 51 - DNA, 20 bp, Artificial Sequence
  Description of Artificial Sequence Synthetic primer
cactcatccgccaccaaagc

SEQ ID NO: 52 - DNA, 20 bp, Artificial Sequence
  Description of Artificial Sequence Synthetic primer
ccttgctcctcctgcctctc

SEQ ID NO: 53 - DNA, 20 bp, Artificial Sequence
  Description of Artificial Sequence Synthetic primer
gacagacagggtgctgtggt

SEQ ID NO: 54 - DNA, 20 bp, Artificial Sequence
  Description of Artificial Sequence Synthetic primer
ttgtagctccgcacgaccat

SEQ ID NO: 55 - DNA, 20 bp, Artificial Sequence
  Description of Artificial Sequence Synthetic primer
atccgccagggcaactactc

SEQ ID NO: 56 - DNA, 20 bp, Artificial Sequence
  Description of Artificial Sequence Synthetic primer
ccacccatccacgagatgct

SEQ ID NO: 57 - DNA, 20 bp, Artificial Sequence
  Description of Artificial Sequence Synthetic primer
cccacccttcccagttcctg

SEQ ID NO: 58 - DNA, 20 bp, Artificial Sequence
  Description of Artificial Sequence Synthetic primer
ggtgcgaacgttggcttgat

SEQ ID NO: 59 - DNA, 20 bp, Artificial Sequence
  Description of Artificial Sequence Synthetic primer
gcttcctcagtccagtcgct

SEQ ID NO: 60 - DNA, 20 bp, Artificial Sequence
  Description of Artificial Sequence Synthetic primer
catgctcggactctctgcca

SEQ ID NO: 61 - DNA, 20 bp, Artificial Sequence
  Description of Artificial Sequence Synthetic primer
cagaacaaggaggcggaggt

SEQ ID NO: 62 - DNA, 20 bp, Artificial Sequence
  Description of Artificial Sequence Synthetic primer
gtttgcgaagcctgggatgg

SEQ ID NO: 63 - DNA, 20 bp, Artificial Sequence
  Description of Artificial Sequence Synthetic primer
cgcgccacacttctctaggt

SEQ ID NO: 64 - DNA, 20 bp, Artificial Sequence
  Description of Artificial Sequence Synthetic primer
acgaaacacggtgggagtgt

SEQ ID NO: 65 - DNA, 20 bp, Artificial Sequence
  Description of Artificial Sequence Synthetic primer
cgtggcgctccttatgggta

SEQ ID NO: 66 - DNA, 20 bp, Artificial Sequence
  Description of Artificial Sequence Synthetic primer
cactggaagcagtgcagcag

SEQ ID NO: 67 - DNA, 20 bp, Artificial Sequence
  Description of Artificial Sequence Synthetic primer
tggtcttgtcggcaggagac

SEQ ID NO: 68 - DNA, 20 bp, Artificial Sequence
  Description of Artificial Sequence Synthetic primer
cccaaagttggtgggccaga

SEQ ID NO: 69 - DNA, 20 bp, Artificial Sequence
  Description of Artificial Sequence Synthetic primer
cctggcctgcaggatgaaga

SEQ ID NO: 70 - DNA, 20 bp, Artificial Sequence
  Description of Artificial Sequence Synthetic primer
cctcggtggaaagagctggt

SEQ ID NO: 71 - DNA, 20 bp, Artificial Sequence
  Description of Artificial Sequence Synthetic primer
tgtgtgcctgccaatcatgc

SEQ ID NO: 72 - DNA, 20 bp, Artificial Sequence
  Description of Artificial Sequence Synthetic primer
acaacccacagttcctgcca

SEQ ID NO: 73 - DNA, 20 bp, Artificial Sequence
  Description of Artificial Sequence Synthetic primer
aaggccaccgagcatctgag

SEQ ID NO: 74 - DNA, 20 bp, Artificial Sequence
  Description of Artificial Sequence Synthetic primer
attctgagcaccgggaaggg

SEQ ID NO: 75 - DNA, 20 bp, Artificial Sequence
  Description of Artificial Sequence Synthetic primer
gcatgctctggctggacttg

SEQ ID NO: 76 - DNA, 20 bp, Artificial Sequence
  Description of Artificial Sequence Synthetic primer
gcttggctggtggcttcttc

SEQ ID NO: 77 - DNA, 20 bp, Artificial Sequence
  Description of Artificial Sequence Synthetic primer
gtcaccatgcgctactccct

SEQ ID NO: 78 - DNA, 20 bp, Artificial Sequence
  Description of Artificial Sequence Synthetic primer
gcagcgatgtctggaagctg

SEQ ID NO: 79 - DNA, 20 bp, Artificial Sequence
  Description of Artificial Sequence Synthetic primer
cctaggccctgggatgtgtg

SEQ ID NO: 80 - DNA, 20 bp, Artificial Sequence
  Description of Artificial Sequence Synthetic primer
gaagggctctcggctcatgt

SEQ ID NO: 81 - RNA, 3000 nt, Artificial Sequence
  Description of Artificial Sequence Synthetic polynucleotide
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 82 - RNA, 180 nt, Artificial Sequence
  Description of Artificial Sequence Synthetic polynucleotide
ggggccggggccggggccggggccggggccggggccggggccggggccggggccggggccggggccggggccggggccggggccggggccggggccggggccggggccggggccggggccggggccggggccggggccggggccggggccggggccggggccggggccggggccggggcc

SEQ ID NO: 83 - DNA, 20 bp, Artificial Sequence
  Description of Artificial Sequence Synthetic primer
gggaggatggtcacctgaaa

SEQ ID NO: 84 - DNA, 20 bp, Artificial Sequence
  Description of Artificial Sequence Synthetic primer
ctggctgtgatgtcagcttc

SEQ ID NO: 85 - DNA, 20 bp, Artificial Sequence
  Description of Artificial Sequence Synthetic primer
ggcatagccctagtcgacac

SEQ ID NO: 86 - DNA, 20 bp, Artificial Sequence
  Description of Artificial Sequence Synthetic primer
cgtaggccaccgaataggag

SEQ ID NO: 87 - DNA, 20 bp, Artificial Sequence
  Description of Artificial Sequence Synthetic primer
tattcaggcctgccgtggta

SEQ ID NO: 88 - DNA, 20 bp, Artificial Sequence
  Description of Artificial Sequence Synthetic primer
ggcacaaagcgactggatga